(19) United States
(12) Patent Application Publication
(10) Pub. No.: US 2025/0215476 A1
(43) Pub. Date: Jul. 3, 2025

(54) EX VIVO PROTEASE ACTIVITY DETECTION FOR HEPATOCELLULAR CARCINOMA

(71) Applicant: Sunbird Bio, Inc., Cambridge, MA (US)

(72) Inventors: Wendy Winckler ADAMOVICH, Melrose, MA (US); Oscar Ajejandro BALBIN, Melrose, MA (US); Benjamin Ray HOLMES, Somerville, MA (US); Faycal TOUTI, Belmont, MA (US)

(21) Appl. No.: 18/852,922

(22) PCT Filed: Mar. 30, 2023
(86) PCT No.: PCT/US2023/016794

§ 371 Date: Sep. 30, 2024

Related U.S. Application Data
(60) Provisional application No. 63/325,249, filed on Mar. 30, 2022.
(60) Provisional application No. 63/331,515, filed on Apr. 15, 2022.

(51) Int. Cl. C12Q 1/37; G01N 33/542; G01N 33/574

(52) U.S. Cl. CPC C12Q 1/37; G01N 33/542; G01N 33/57488; G01N 2333/471; G01N 2333/948; G01N 2333/96425; G01N 2333/96433; G01N 2333/96455; G01N 2333/96486

(57) ABSTRACT

The present application provides compositions and methods for determining hepatocellular carcinoma (HCC) in a subject. The method comprises contacting a body fluid with a molecule comprising a reporter thereof and the reported is cleaved by an agent in the body fluid. Wherein, the subject has or is suspected of having cirrhosis of a liver.

### CROSS-REFERENCE

This application claims the benefit of U.S. Provisional Application No. 63/325,249, filed Mar. 30, 2022, and U.S. Provisional Application No. 63/331,515, filed Apr. 15, 2022, both of which are herein entirely incorporated by reference.

### BRIEF SUMMARY

Provided herein is a method comprising contacting a body fluid sample from a subject with a synthetic molecule, wherein the contacting occurs ex vivo, wherein the subject has or is suspected of having cirrhosis of a liver of the subject, wherein the synthetic molecule comprises a cleavable linker, a reporter, and a carrier, and wherein the synthetic molecule reacts with an enzyme from the body fluid, causing the reporter to form a detectable signal, and detecting the detectable signal, wherein the detection indicates whether the subject has hepatocellular carcinoma (HCC), wherein the detection comprises a detection sensitivity of at least 0.5.

In some embodiments, the detection sensitivity is at least 0.8. In some embodiments, the detection sensitivity is calculated by dividing a number of samples with HCC and identified to be positive by a total number of samples, wherein the total number of samples comprises the sum of the samples with HCC and identified to be positive and samples with HCC and identified to be negative.

In some embodiments, wherein the HCC comprises a stage of HCC. In some embodiments, the enzyme from the body fluid cleaves the cleavable linker, thereby releasing the reporter from the synthetic molecule, wherein the released reporter forms the detectable signal. In some embodiments, the cleavable linker comprises a peptide.

In some embodiments, the synthetic molecule comprises a panel of peptides. In some embodiments, the panel of peptides comprises at least 10 different peptides. In some embodiments, the enzyme comprises a protease. In some embodiments, the protease comprises a matrix metalloprotease (MMP), a dipeptidyl-peptidase 4 (DPP4), an alanyl aminopeptidase (ANPEP), Aminopeptidase (ERAP1, ARTS-1), a Kallikrein related peptidase 14 (KLK14), a Cathepsin or an ADAM17.

In some embodiments, the reporter comprises a fluorescent label. In some embodiments, the fluorescent label comprises a 5-carboxyfluorescein (5-FAM), a 7-amino-4-carbamoylmethylcoumarin (ACC), a 7-Amino-4-methylcoumarin (AMC), a 2-Aminobenzoyl (Abz), a Cy7, a Cy5, a Cy3, or a (5-((2-Aminoethyl)amino)naphthalene-1-sulfonic acid) (EDANS). In some embodiments, the synthetic molecule further comprises a fluorescent quencher. In some embodiments, the fluorescent quencher comprises BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl]azo)benzoyl), Dnp (2,4-dinitrophenyl), or Eclipse.

In some embodiments, the body fluid sample comprises blood, plasma, bone marrow fluid, lymphatic fluid, bile, amniotic fluid, mucosal fluid, saliva, urine, cerebrospinal fluid, spinal fluid, synovial fluid, semen, ductal aspirate, feces, stool, vaginal effluent, lachrymal fluid, tissue lysate or patient-derived cell line supernatant. In some embodiments, the body fluid sample comprises a rinse fluid, a conditioning media or buffer, a swab viral transport media, a saline, a culture media, or a cell culture supernatant. In some embodiments, the rinse fluid comprises a mouthwash rinse, a bronchioalveolar rinse, a lavage fluid, a hair wash rinse, a nasal spray effluent, a swab of any bodily surface, orifice, organ structure, or solid tumor biopsies applied to saline or any media, or any derivatives thereof.

In some embodiments, the body fluid sample comprises an alpha fetoprotein (AFP) level of less than about 10 ng/ml. in some embodiments, the detection further comprises detecting the AFP level of the body fluid sample. In some embodiment, the detection comprises detecting a rate of formation or an amount of the detectable signal. In some embodiments, the subject comprises a mammal. In some embodiments, the subject comprises a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIGURE.” or “FIGURES.” herein), of which:

FIG. 1 shows a plurality of probes according to the current application. Each probe 101 includes a reporter 103, shown as a star in FIG. 1. The reporters 103, are linked to a cleavable linker 105, which is a cleavable substrate for an agent 107.

FIG. 2 shows cleavage of the reporter in a plurality of the probes. As shown, cleavage by the agent 107 of the cleavable linker 105 results in the reporters 103 being cleaved from the probe 101. Once cleaved, the cleaved reporters 203 can be detected and/or distinguished from un-cleaved reporters 103. The presence and detection of cleaved reporters 203 indicates that the agents 107 are present and active in a sample. In addition, the absence of an agent activity may be used for detection associated with a decrease in activity. The activity of the agents can be quantified based on, for example, the rate at which the cleavage reaction takes place or the amount of cleaved reporters in a sample or by other means such as a ratio of rates against an appropriate control or a ratio of cleaved reporters against an appropriate control.

FIG. 3 illustrates a method 301 of evaluating a biological condition in a subject using the probes 101.

FIG. 4 shows the selection of probes to use in a composition to analyze the activities of agents to analyze one or more particular, biological conditions or disease states. The activity of one or more agents may be associated with a biological condition or disease state. This may include the progression of a particular condition or state over time. Thus, to evaluate a biological condition or disease state in a subject, probes that can be cleaved by agents of interest are selected from the library for inclusion in a condition-specific panel 403. The selected probes 405 of the condition-specific panel are differentially labeled so that the activity of the predetermined proteases can be measured 305. The different probes 101, including those included in library 401, may include features that confer properties to the fragments that ensure accurate, multiplex detection of agent activity. Such properties include, for example improved cleavage, detection, solubility, stability, reproducibility, robustness and/or expanded compatibility with different types of reporter.

FIG. 5 and FIG. 6 show that protease activity significantly discriminates Hepatocellular carcinoma (HCC) samples vs healthy samples in two types of samples—viral-induced HCC (FIG. 5) and Western (metabolically driven) HCC (FIG. 6).

FIG. 7 shows that protease activity significantly discriminates HCC samples vs cirrhosis samples.

FIG. 8 shows that HCC signal generalizes across sample cohorts without classifier retraining, increasing confidence in results.

FIG. 9A-9B shows that protease activity varies by cancer stage in HCC in both viral-induced HCC (FIG. 9A) and in lifestyle-induced HCC (FIG. 9B).

FIG. 10A-10B show results of AFP-protease biosensor panel assays. FIG. 10A shows results of the classifier cross-validated in low AFP patients (AFP<10 ng/ml). FIG. 10B shows results of the combined classifier using protease panel+AFP.

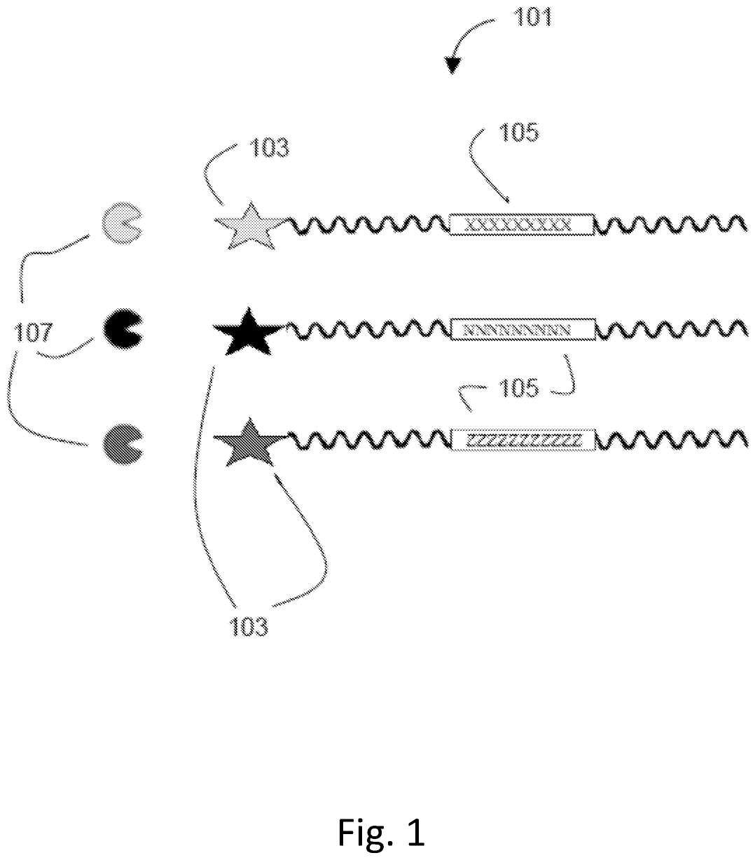

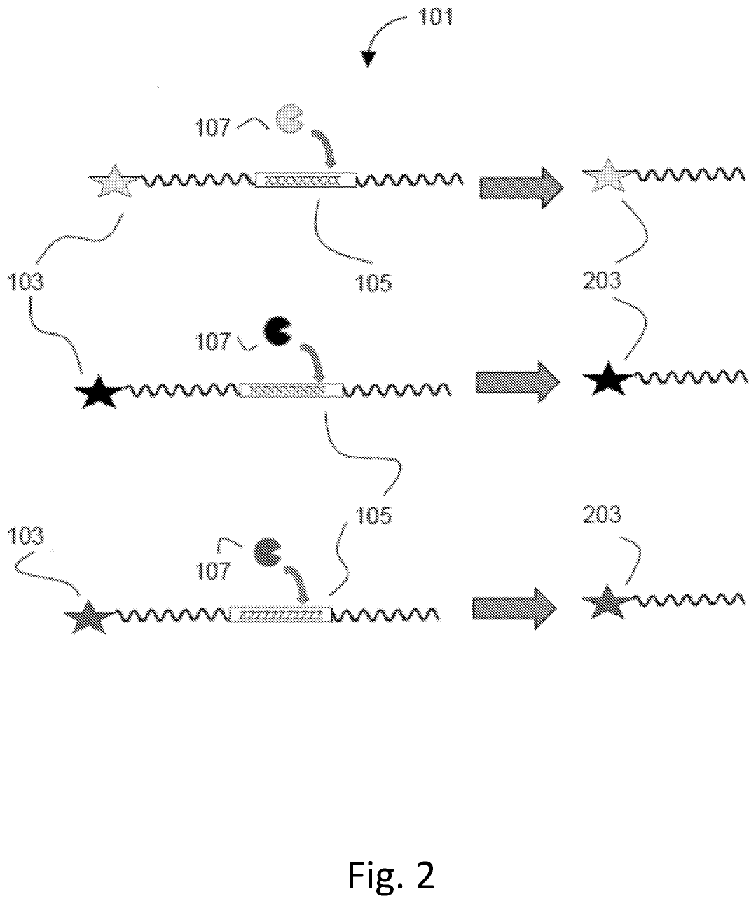

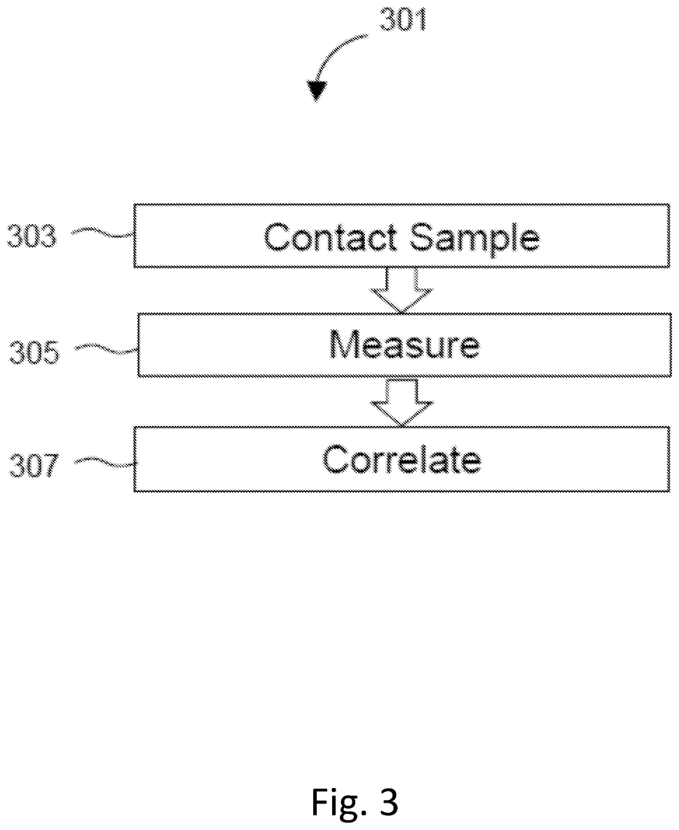

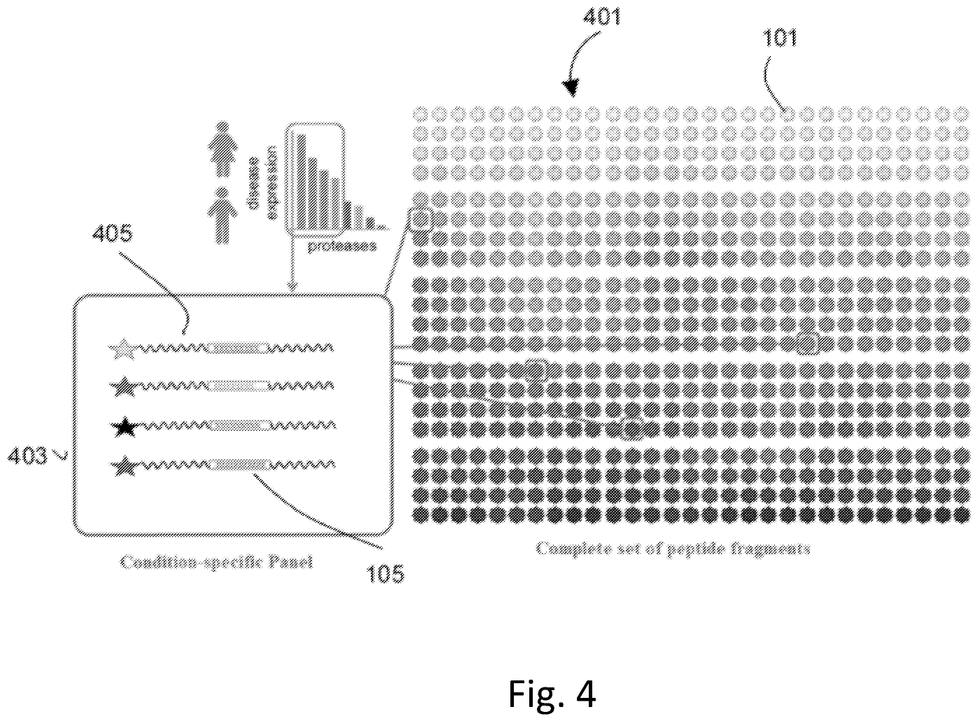

### DETAILED DESCRIPTION

Provided herein are methods comprising contacting a body fluid sample from a subject with a synthetic molecule (also referred to herein as a/the “molecule” or a/the “probe”) ex vivo, in vitro, or in vivo. In some embodiments, the molecule comprises a cleavable linker and a reporter. The cleavable linker is cleaved by an agent from the body fluid, releasing the reporter from the molecule. In some embodiments, the method further comprises detecting a rate of formation or an amount of the released reporter. In some embodiments, the rate of formation or amount of the released report is significantly different from a healthy subject. In some embodiments, the body fluid comprises plasma. In some embodiments, the method further comprises determining a disease or condition of the subject based on the detection. In some embodiments, the disease or condition comprises hepatocellular carcinoma (HCC). In some embodiments, the subject has or is suspected of having cirrhosis of liver. In some embodiments, the method comprises distinguishing subjects having HCC from subjects having only cirrhosis of the liver.

In one aspect, the body fluid sample is contacted by a second molecule with a second cleavable linker and a second reporter. In some embodiments, the second cleavable linker is cleaved by a second agent from the body fluid, releasing the second reporter from the second molecule. In some embodiments, the method further comprises detecting a rate of formation or an amount of the second released reporter. In some embodiments, the method further comprises determining a disease or condition of the subject based on the detection of the first released reporter and the detection of the second released reporter. In some embodiments, the method described herein can be used in a multiplexed format, such that a single body fluid sample can be used to ascertain the activity of multiple, select agents. This allows diagnostic panels to be created for specific pathologies and conditions, which leverage the activity of multiple agents to provide a more complete and accurate assessment of a certain condition. These panels can be used to correlate the activity of multiple agents with a particular condition or disease-state. These signatures can be saved, for example, in a database and used to assess the conditions or disease-state for subsequent individuals assessed by a particular protease activity panel. In some embodiments, a classification tool is used in the analysis to differentiate between healthy and diseased patients, or between discrete stages of disease. The classification tool may be supervised Machine Learning classification algorithms including, but not limited, to Logistic Regression, Naive Bayes, Support Vector Machine, Random Forest, Gradient Boosting or Neural Networks. Furthermore, if the modeled variable is continuous in nature (e.g., tumor volume), one could use continuous regression approaches such as Ridge Regression, Kernel Ridge Regression, or Support Vector Regression. These algorithms would operate on the multi-dimensional feature space defined by the measurements of multiple probes (or a mathematical function of those measurements such as probe ratios) in order to learn the relationship between probe measurements and disease status. Finally, one could combine probe measurements with clinical variables such as age, gender, or patients' comorbid status. In that case, one could either incorporate clinical features in the classifier directly or, alternatively, learn a second-order classifier which combines a probe-only prediction with clinical features to produce a result that is calibrated for those variables.

In some embodiments, the disease or condition comprises a liver disease, a cancer, an organ transplant rejection, an infectious disease, an allergic disease, an autoimmunity and a chronic inflammation. In some embodiments, the liver disease comprises a certain fibrosis stage or a certain nonalcoholic fatty liver disease activity score (NAS) of Non-alcoholic steatohepatitis (NASH).

In another aspect, the methods described herein comprises ex vivo, multiplex detection of enzyme activity to diagnose and monitor pathologies and treatments in a subject. That enzyme activity can be used to diagnose and monitor a disease and condition in an internal organ of the subject.

### Detection Probe/Molecule

Determination of the disease or condition is based on the rate of formation or amount of the released reporter detected in the sample. A probe/molecule is introduced to the body fluid samples. The probe/molecule comprises a cleavable linker and a reporter, and an agent of from the body fluid cleave the cleavable linker, releasing a cleaved reporter. The probe/molecule may have any structure that can fulfill this function. In some embodiments, the reporter may be covalently linked to a cleavable linker. In some embodiments, the reporter comprises a fluorescent label, a mass tag, a chromophore, an electrochemically active molecule, a bio-Layer interferometry or surface plasmon resonance detectable molecule, a precipitating substance, a mass spectrometry and liquid chromatography substrate (including size exclusion, reverse phase, isoelectric point, etc.), a magnetically active molecule, a gel forming and/or viscosity changing molecule, an immunoassay detectable molecule, a cell-based amplification detectable molecule, a nucleic acid barcode, or any combinations thereof.

In some embodiments, the reporter comprises a fluorescent label and the molecule also comprises a quencher. In some embodiments, the quencher is covalently linked to the cleavable linker. In some embodiments an internally quenched fluorophore is linked to the cleavable linker. In some embodiments, the molecule further comprises a self-immolative spacer. In some other embodiments, the molecule further comprises a carrier.

### Cleavable Linker

In some aspects, the probe/molecule described herein comprises a cleavable linker. The cleavable linker as described herein may be in any structure that is capable of being cleaved by an agent. In some embodiments, the cleavable linker comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker, an auto-immolable linker (three component probe=enzyme substrate+linker+reporter) or a combination thereof. In some embodiments, the reporter can be in an inactive form and under disease activity becomes detectable. Geoffray Leriche, Louise Chisholm, Alain Wagner, Cleavable linkers in chemical biology, Bioorganic & Medicinal Chemistry, Volume 20, Issue 2, 2012, Pages 571-582, ISSN 0968-0896, https://doi.org/10.1016/j.bmc.2011.07.048.

Cross-linking agents aim to form a covalent bond between two spatially adjacent residues within one or two polymer chains. To identify protein binding partners, the cross-linking agents need to be able to detect and stabilize transient interactions. The crosslinking agents frequently form covalent links between lysine or cysteine residues in the proteins. Alternatively, the cross-linking agent can be photoreactive. Cross-linking cleavable linkers can be used to distinguish between inter- and intra-protein interactions of receptors, signaling cascades, and the structure of multi-protein complexes.

In some embodiments, the cleavable linker comprises a peptide. The core structure of a peptide linker sometimes comprises of either a di-peptide or a tetra-peptide that is recognized and cleaved by lysosomal enzymes. Proteases (also called peptidases) catalyze the breakdown of peptide bonds by hydrolysis and is restricted to a specific sequence of amino acids recognizable by the proteases. Commonly used proteases comprise pepsin, trypsin or chymotrypsin. Since proteases have key roles in many diseases, peptide linkers are widely used in drug release systems or in diagnostic tools. In some embodiments, the peptide linkers comprise a short peptide sequence. In some embodiments, the peptide linkers comprise at least one non-naturally (also referred to as “unnaturally”) occurring amino acid.

In some embodiments, the peptide linkers comprise less than about 20 amino acids in length. In some embodiments, the peptide linkers comprise between 10 and 100 amino acids in length. In some embodiments, the peptide linkers comprise 1 to 5, 1 to 10, 1 to 20, 1 to 30, 1 to 50, 1 to 70, 1 to 90, 1 to 100, 5 to 10, 5 to 20, 5 to 30, 5 to 50, 5 to 70, 5 to 90, 5 to 100, 10 to 20, 10 to 30, 10 to 50, 10 to 70, 10 to 90, 10 to 100, 20 to 30, 20 to 50, 20 to 70, 20 to 90, 20 to 100, 30 to 50, 30 to 70, 30 to 90, 30 to 100, 50 to 70, 50 to 90, 50 to 100, 70 to 90, 70 to 100, or 90 to 100 amino acids in length.

TABLE 1
Example sequences for peptide linkers and corresponding
probe construct designs

| SEQ | | | | SEQ |
|---|---|---|---|---|
| ID | | Exemplary | Exemplary probe | ID |
| NO | Sequence | probe name | construct | NO: |
| 1 | SGRSG | Probe #1 | 5-FAM-GSGRSGGK(CPQ2)-PEG2-kk-GC | 678 |
| | | | | and |
| | | | | 1297 |
| 2 | PGPREG | Probe #2 | 5-FAM-GPGPREGGK(CPQ2)-PEG2-kk-GC | 679 |
| | | | | and |
| | | | | 1297 |
| 3 | IEPDSGSQ | Probe #3 | 5-FAM-GIEPDSGSQGK(CPQ2)-PEG2-kk-GC | 680 |
| | | | | and |
| | | | | 1297 |
| 4 | VVADSSMES | Probe #4 | 5-FAM-GVVADSSMESGK(CPQ2)-PEG2-kk- | 681 |
| | | | GC | and |
| | | | | 1297 |
| 5 | PTSY | Probe #5 | 5-FAM-GPTSYGK(CPQ2)-PEG2-kk-GC | 682 |
| | | | | and |
| | | | | 1297 |
| 6 | YRFK | Probe #6 | 5-FAM-GYRFKGK(CPQ2)-PEG2-kk-GC | 683 |
| | | | | and |
| | | | | 1297 |
| 7 | KVPL | Probe #7 | 5-FAM-GKVPLGK(CPQ2)-PEG2-kk-GC | 684 |
| | | | | and |
| | | | | 1297 |
| 8 | VDVAD | Probe #8 | 5-FAM-GVDVADGK(CPQ2)-PEG2-kk-GC | 685 |
| | | | | and |
| | | | | 1297 |
| 9 | LETD | Probe #9 | 5-FAM-GLETDGK(CPQ2)-PEG2-kk-GC | 686 |
| | | | | and |
| | | | | 1297 |
| 10 | LEHD | Probe #10 | 5-FAM-GLEHDGK(CPQ2)-PEG2-kk-GC | 687 |
| | | | | and |
| | | | | 1297 |
| 11 | REQD | Probe #11 | 5-FAM-GREQDGK(CPQ2)-PEG2-kk-GC | 688 |
| | | | | and |
| | | | | 1297 |
| 12 | DEVD | Probe #12 | 5-FAM-GDEVDGK(CPQ2)-PEG2-kk-GC | 689 |
| | | | | and |
| | | | | 1297 |
| 13 | VEID | Probe #13 | 5-FAM-GVEIDGK(CPQ2)-PEG2-kk-GC | 690 |
| | | | | and |
| | | | | 1297 |
| 14 | VQVDGW | Probe #14 | 5-FAM-GVQVDGWGK(CPQ2)-PEG2-kk-GC | 691 |
| | | | | and |
| | | | | 1297 |
| 15 | YEVDGW | Probe #15 | 5-FAM-GYEVDGWGK(CPQ2)-PEG2-kk-GC | 692 |
| | | | | and |
| | | | | 1297 |
| 16 | LEVD | Probe #16 | 5-FAM-GLEVDGK(CPQ2)-PEG2-kk-GC | 693 |
| | | | | and |
| | | | | 1297 |
| 17 | IEVE | Probe #17 | 5-FAM-GIEVEGK(CPQ2)-PEG2-kk-GC | 694 |
| | | | | and |
| | | | | 1297 |
| 18 | AAPV | Probe #18 | 5-FAM-GAAPVGK(CPQ2)-PEG2-kk-GC | 695 |
| | | | | and |
| | | | | 1297 |
| 19 | FFKF | Probe #19 | 5-FAM-GFFKFGK(CPQ2)-PEG2-kk-GC | 696 |
| | | | | and |
| | | | | 1297 |
| 20 | GRRGKGG | Probe #20 | 5-FAM-GGRRGKGGGK(CPQ2)-PEG2-kk- | 697 |
| | | | GC | and |
| | | | | 1297 |
| 21 | VKKR | Probe #21 | 5-FAM-GVKKRGK(CPQ2)-PEG2-kk-GC | 698 |
| | | | | and |
| | | | | 1297 |
| 22 | FAAF(NO2)FVL | Probe #22 | 5-FAM-GFAAF(NO2)FVL GK(CPQ2)-PEG2- | 699 |
| | | | kk-GC | and |
| | | | | 1297 |
| | VVR | Probe #23 | 5-FAM-GVVRGK(CPQ2)-PEG2-kk-GC | 700 |
| | | | | and |
| | | | | 1297 |
| 24 | KQKLR | Probe #24 | 5-FAM-GKQKLRGK(CPQ2)-PEG2-kk-GC | 701 |
| | | | | and |
| | | | | 1297 |
| 25 | RPPGFSAF | Probe #25 | 5-FAM-GRPPGFSAFGK(CPQ2)-PEG2-kk- | 702 |
| | | | GC | and |
| | | | | 1297 |
| | GPR | Probe #26 | 5-FAM-GGPRGK(CPQ2)-PEG2-kk-GC | 703 |
| | | | | and |
| | | | | 1297 |
| | FR | Probe #27 | 5-FAM-GFRGK(CPQ2)-PEG2-kk-GC | 704 |
| | | | | and |
| | | | | 1297 |
| 28 | LPLGL | Probe #28 | 5-FAM-GLPLGLGK(CPQ2)-PEG2-kk-GC | 705 |
| | | | | and |
| | | | | 1297 |
| 29 | KPLGL | Probe #29 | 5-FAM-GKPLGLGK(CPQ2)-PEG2-kk-GC | 706 |
| | | | | and |
| | | | | 1297 |
| 30 | (Gaba)PQGLE | Probe #30 | 5-FAM-G(Gaba)PQGLE GK(CPQ2)-PEG2- | 707 |
| | | | kk-GC | and |
| | | | | 1297 |
| 31 | PKPLAL | Probe #31 | 5-FAM-GPKPLALGK(CPQ2)-PEG2-kk-GC | 708 |
| | | | | and |
| | | | | 1297 |
| 32 | GPSGIHV | Probe #32 | 5-FAM-GGPSGIHVGK(CPQ2)-PEG2-kk-GC | 709 |
| | | | | and |
| | | | | 1297 |
| 33 | WAHRTTFYRR | Probe #33 | 5-FAM-GWAHRTTFYRRGAGK(CPQ2)- | 710 |
| | GA | | PEG2-kk-GC | and |
| | | | | 1297 |
| 34 | WKLRSSKQ | Probe #34 | 5-FAM-GWKLRSSKQGK(CPQ2)-PEG2-kk- | 711 |
| | | | GC | and |
| | | | | 1297 |
| | PFR | Probe #35 | 5-FAM-GPFRGK(CPQ2)-PEG2-kk-GC | 712 |
| | | | | and |
| | | | | 1297 |
| 36 | SYRIF | Probe #36 | 5-FAM-GSYRIFGK(CPQ2)-PEG2-kk-GC | 713 |
| | | | | and |
| | | | | 1297 |
| | RPY | Probe #37 | 5-FAM-GRPYGK(CPQ2)-PEG2-kk-GC | 714 |
| | | | | and |
| | | | | 1297 |
| 38 | TAFRSAYG | Probe #38 | 5-FAM-GTAFRSAYGGK(CPQ2)-PEG2-kk- | 715 |
| | | | GC | and |
| | | | | 1297 |
| 39 | WAAFRFSQA | Probe #39 | 5-FAM-GWAAFRFSQAGK(CPQ2)-PEG2-kk- | 716 |
| | | | GC | and |
| | | | | 1297 |
| | VPR | Probe #40 | 5-FAM-GVPRGK(CPQ2)-PEG2-kk-GC | 717 |
| | | | | and |
| | | | | 1297 |
| | G | Probe #41 | 5-FAM-GGK(CPQ2)-PEG2-kk-GC | 1297 |
| 42 | KLRSSKQ | Probe #42 | 5-FAM-GKLRSSKQGK(CPQ2)-PEG2-kk-GC | 718 |
| | | | | and |
| | | | | 1297 |
| 43 | YASR | Probe #43 | 5-FAM-GYASRGK(CPQ2)-PEG2-kk-GC | 719 |
| | | | | and |
| | | | | 1297 |
| 44 | RFAQAQQQLP | Probe #44 | 5-FAM-GRFAQAQQQLPGK(CPQ2)-PEG2- | 720 |
| | | | kk-GC | and |
| | | | | 1297 |
| 45 | KPAKFFRL | Probe #45 | 5-FAM-GKPAKFFRLGK(CPQ2)-PEG2-kk- | 721 |
| | | | GC | and |
| | | | | 1297 |
| 46 | PRAAA(hF)TSP | Probe #46 | 5-FAM-GPRAAA(hF)TSPGK(CPQ2)-PEG2- | 722 |
| | | | kk-GC | and |
| | | | | 1297 |
| 47 | VGPQRFSGAP | Probe #47 | 5-FAM-GVGPQRFSGAPGK(CPQ2)-PEG2- | 723 |
| | | | kk-GC | and |
| | | | | 1297 |
| 48 | FFLAQA(hF)RS | Probe #48 | 5-FAM-GFFLAQA(hF)RS GK(CPQ2)-PEG2- | 724 |
| | | | kk-GC | and |
| | | | | 1297 |
| 49 | PLAQAV | Probe #49 | 5-FAM-GPLAQAVGK(CPQ2)-PEG2-kk-GC | 725 |
| | | | | and |
| | | | | 1297 |
| 50 | RTAAVFRP | Probe #50 | 5-FAM-GRTAAVFRPGK(CPQ2)-PEG2-kk- | 726 |
| | | | GC | and |
| | | | | 1297 |
| 51 | DVQEFRGVTA | Probe #51 | 5-FAM-GDVQEFRGVTAVIRGK(CPQ2)- | 727 |
| | VIR | | PEG2-kk-GC | and |
| | | | | 1297 |
| | TEGEARGSVI | Probe #52 | 5-FAM-GTEGEARGSVIGK(CPQ2)-PEG2- | 728 |
| | | | kk-GC | and |
| 52 | | | | 1297 |
| | 1-TR | Probe #53 | 5-FAM-G-1-TRGK(CPQ2)-PEG2-kk-GC | 729 |
| | | | | and |
| | | | | 1297 |
| 54 | PLFAERK | Probe #54 | 5-FAM-GPLFAERKGK(CPQ2)-PEG2-kk-GC | 730 |
| | | | | and |
| | | | | 1297 |
| 55 | LLVY | Probe #55 | 5-FAM-GLLVYGK(CPQ2)-PEG2-kk-GC | 731 |
| | | | | and |
| | | | | 1297 |
| 56 | QQKRKIVL | Probe #56 | 5-FAM-GQQKRKIVLGK(CPQ2)-PEG2-kk- | 732 |
| | | | GC | and |
| | | | | 1297 |
| 57 | ASHLGLAR | Probe #57 | 5-FAM-GASHLGLARGK(CPQ2)-PEG2-kk- | 733 |
| | | | GC | and |
| | | | | 1297 |
| 58 | LPSRSSKI | Probe #58 | 5-FAM-GLPSRSSKIGK(CPQ2)-PEG2-kk-GC | 734 |
| | | | | and |
| | | | | 1297 |
| 59 | STGRNGFK | Probe #59 | 5-FAM-GSTGRNGFKGK(CPQ2)-PEG2-kk- | 735 |
| | | | GC | and |
| | | | | 1297 |
| 60 | SLLRSEET | Probe #60 | 5-FAM-GSLLRSEETGK(CPQ2)-PEG2-kk-GC | 736 |
| | | | | and |
| | | | | 1297 |
| 61 | HRGRTLEI | Probe #61 | 5-FAM-GHRGRTLEIGK(CPQ2)-PEG2-kk- | 737 |
| | | | GC | and |
| | | | | 1297 |
| 62 | YLGRSYKV | Probe #62 | 5-FAM-GYLGRSYKVGK(CPQ2)-PEG2-kk- | 738 |
| | | | GC | and |
| | | | | 1297 |
| 63 | EKQRIIGG | Probe #63 | 5-FAM-GEKQRIIGGGK(CPQ2)-PEG2-kk-GC | 739 |
| | | | | and |
| | | | | 1297 |
| 64 | QRQRIIGG | Probe #64 | 5-FAM-GQRQRIIGGGK(CPQ2)-PEG2-kk- | 740 |
| | | | GC | and |
| | | | | 1297 |
| 65 | LORIYK | Probe #65 | 5-FAM-GLQRIYKGK(CPQ2)-PEG2-kk-GC | 741 |
| | | | | and |
| | | | | 1297 |
| 66 | SLGRKIQI | Probe #66 | 5-FAM-GSLGRKIQIGK(CPQ2)-PEG2-kk-GC | 742 |
| | | | | and |
| | | | | 1297 |
| 67 | HAAPRSADIQI | Probe #67 | 5-FAM-GHAAPRSADIQIDIGK(CPQ2)- | 743 |
| | DI | | PEG2-kk-GC | and |
| | | | | 1297 |
| | FGR | Probe #68 | 5-FAM-GFGRGK(CPQ2)-PEG2-kk-GC | 744 |
| | | | | and |
| | | | | 1297 |
| 69 | SLGR | Probe #69 | 5-FAM-GSLGRGK(CPQ2)-PEG2-kk-GC | 745 |
| | | | | and |
| | | | | 1297 |
| 70 | GLQR | Probe #70 | 5-FAM-GGLQRGK(CPQ2)-PEG2-kk-GC | 746 |
| | | | | and |
| | | | | 1297 |
| 71 | SVARTLLV | Probe #71 | 5-FAM-GSVARTLLVGK(CPQ2)-PEG2-kk- | 747 |
| | | | GC | and |
| | | | | 1297 |
| 72 | GRIFG | Probe #72 | 5-FAM-GGRIFGGK(CPQ2)-PEG2-kk-GC | 748 |
| | | | | and |
| | | | | 1297 |
| | APK | Probe #73 | 5-FAM-GAPKGK(CPQ2)-PEG2-kk-GC | 749 |
| | | | | and |
| | | | | 1297 |
| 74 | GFSPY | Probe #74 | 5-FAM-GGFSPYGK(CPQ2)-PEG2-kk-GC | 750 |
| | | | | and |
| | | | | 1297 |
| 75 | WELRHAGH | Probe #75 | 5-FAM-GWELRHAGHGK(CPQ2)-PEG2-kk- | 751 |
| | | | GC | and |
| | | | | 1297 |
| 76 | RQSRIVGGE | Probe #76 | 5-FAM-GRQSRIVGGEGK(CPQ2)-PEG2-kk- | 752 |
| | | | GC | and |
| | | | | 1297 |
| 77 | EQAVYQTI | Probe #77 | 5-FAM-GEQAVYQTIGK(CPQ2)-PEG2-kk- | 753 |
| | | | GC | and |
| | | | | 1297 |
| 78 | VAYSGENTFG | Probe #78 | 5-FAM-GVAYSGENTFGFGK(CPQ2)-PEG2- | 754 |
| | F | | kk-GC | and |
| | | | | 1297 |
| | GGR | Probe #79 | 5-FAM-GGGRGK(CPQ2)-PEG2-kk-GC | 755 |
| | | | | and |
| | | | | 1297 |
| 80 | ATAD | Probe #80 | 5-FAM-GATADGK(CPQ2)-PEG2-kk-GC | 756 |
| | | | | and |
| | | | | 1297 |
| 81 | RPLESNAV | Probe #81 | 5-FAM-GRPLESNAVGK(CPQ2)-PEG2-kk- | 757 |
| | | | GC | and |
| | | | | 1297 |
| 82 | RPLGLAR | Probe #82 | 5-FAM-GRPLGLARGK(CPQ2)-PEG2-kk-GC | 758 |
| | | | | and |
| | | | | 1297 |
| 83 | AAFF | Probe #83 | 5-FAM-GAAFFGK(CPQ2)-PEG2-kk-GC | 759 |
| | | | | and |
| | | | | 1297 |
| 84 | RVKRGLA | Probe #84 | 5-FAM-GRVKRGLAGK(CPQ2)-PEG2-kk-GC | 760 |
| | | | | and |
| | | | | 1297 |
| | AAL | Probe #85 | 5-FAM-GAALGK(CPQ2)-PEG2-kk-GC | 761 |
| | | | | and |
| | | | | 1297 |
| 86 | CGGmeGVndne | Probe #86 | 5-FAM-CGGmeGVndneeGFFsArGK(CPQ2) | 762 |
| | eGFFsAr | | | |
| 87 | GPQGIWGQ | Probe #87 | 5FAM-GGPQGIWGQK(CPQ2)-PEG2-C | 763 |
| 88 | GLVPRGS | Probe #88 | 5FAM-GGLVPRGSGK(CPQ2)-PEG2-C | 764 |
| 89 | GPVGLI | Probe #89 | 5FAM-GGPVGLIGK(CPQ2)-PEG2-C | 765 |
| 90 | GPWGIWGQ | Probe #90 | 5FAM-GGPWGIWGQGK(CPQ2)-PEG2-C | 766 |
| 91 | GPVPLSLVM | Probe #91 | 5FAM-GGPVPLSLVMK(CPQ2)-PEG2-C | 767 |
| 92 | Gf-Pip-RSGG | Probe #92 | 5FAM-GGf-Pip-RSGGGK(CPQ2)-PEG2-C | 768 |
| 93 | PLGMRG | Probe #93 | 5FAM-GGf-Pip-KSGGGK(CPQ2)-PEG2-C | 769 |
| 94 | PLGMRG | Probe #94 | (FAM)-GPLGMRGG-K(CPQ2)-PEG2-k-GC | 770 |
| 95 | P-(Cha)-G- | Probe #95 | (FAM)-GP-(Cha)-G-Cys(Me)-HAG-K(CPQ2)- | 771 |
| | Cys(Me)-HA | | PEG2-kk-GC | and |
| | | | | 1297 |
| 96 | RPLALWESQ | Probe #96 | (FAM)-GRPLALWESQG-K(CPQ2)-PEG2-k- | 772 |
| | | | GC | |
| 97 | SGKGPRQITA | Probe #97 | (FAM)-SGKGPRQITA-K(CPQ2)-PEG2-k-GC | 773 |
| 98 | SGPLFYSVTA | Probe #98 | (FAM)-SGPLFYSVTA-K(CPQ2)-PEG2-kk- | 774 |
| | | | GC | and |
| | | | | 1297 |
| 99 | SGRIFLRTA | Probe #99 | (FAM)-SGRIFLRTA-K(CPQ2)-PEG2-GC | 775 |
| 100 | SGRSENIRTA | Probe #100 | (FAM)-SGRSENIRTA-K(CPQ2)-PEG2-GC | 776 |
| 101 | GSGGS | Probe #101 | (FAM)-GGSGGS-K(CPQ2)-PEG2-kk-GC | 777 |
| | | | | and |
| | | | | 1297 |
| 102 | KPILFFRLKG | Probe #102 | (FAM)-GKPILFFRLKG-K(CPQ2)-PEG2-kk- | 778 |
| | | | GC | and |
| | | | | 1297 |
| 103 | AWESR(Nle) | Probe #103 | (FAM)-GAWESR(Nle)GK(CPQ2)-NH2 | 779 |
| 104 | NEKSG(Nle) | Probe #104 | (FAM)-GNEKSG(Nle)GK(CPQ2)-NH2 | 780 |
| 105 | NATIVY | Probe #105 | (FAM)-GNATIVYGK(CPQ2)-PEG2-k-NH2 | 781 |
| 106 | DPFVVS | Probe #106 | (FAM)-GDPFVVSGK(CPQ2)-PEG2-k-NH2 | 782 |
| 107 | FH(Nle)FTK | Probe #107 | (FAM)-GFH(Nle)FTKGK(CPQ2)-PEG2-k- | 783 |
| | | | NH2 | |
| 108 | (Nle)NWHKH | Probe #108 | (FAM)-G(Nle)NWHKHGK(CPQ2)-NH2 | 784 |
| 109 | FARRWG | Probe #109 | (FAM)-GFARRWGGK(CPQ2)-PEG2-k-NH2 | 785 |
| 110 | PGKWSK | Probe #110 | (FAM)-GPGKWSKGK(CPQ2)-PEG2-k-NH2 | 786 |
| 111 | YEEAQP | Probe #111 | (FAM)-GYEEAQPGK(CPQ2)-PEG2-k-NH2 | 787 |
| 112 | YGAIKK | Probe #112 | (FAM)-GYGAIKKGK(CPQ2)-PEG2-k-NH2 | 788 |
| 113 | TS(Nle)EGY | Probe #113 | (FAM)-GTS(Nle)EGYGK(CPQ2)-PEG2-k | 789 |
| 114 | PNNFGS | Probe #114 | (FAM)-GPNNFGSGK(CPQ2)-PEG2-k-NH2 | 790 |
| 115 | EDTRNT | Probe #115 | (FAM)-GEDTRNTGK(CPQ2)-NH2 | 791 |
| 116 | KDLEQS | Probe #116 | (FAM)-GKDLEQSGK(CPQ2)-NH2 | 792 |
| 117 | AALHND | Probe #117 | (FAM)-GAALHNDGK(CPQ2)-PEG2-kk-NH2 | 793 |
| 118 | ADSFFK | Probe #118 | (FAM)-GADSFFKGK(CPQ2)-NH2 | 794 |
| 119 | ITFWRA | Probe #119 | (FAM)-GITFWRAGK(CPQ2)-NH2 | 795 |
| 120 | LSD(Nle)RL | Probe #120 | (FAM)-GLSD(Nle)RLGK(CPQ2)-NH2 | 796 |
| 121 | EVGWTY | Probe #121 | (FAM)-GEVGWTYGK(CPQ2)-PEG2-k-NH2 | 797 |
| 122 | IAFRQ(Nle) | Probe #122 | (FAM)-GIAFRQ(Nle)GK(CPQ2)-NH2 | 798 |
| 123 | YNIHT(Nle) | Probe #123 | (FAM)-GYNIHT(Nle)GK(CPQ2)-PEG2-kk- | 799 |
| | | | NH2 | |
| 124 | (Nle)LWANH | Probe #124 | (FAM)-G(Nle)LWANHGK(CPQ2)-PEG2-kk- | 800 |
| | | | NH2 | |
| 125 | LYSVQV | Probe #125 | (FAM)-GLYSVQVGK(CPQ2)-PEG2-k-NH2 | 801 |
| 126 | SHI(Nle)SN | Probe #126 | (FAM)-GSHI(Nle)SNGK(CPQ2)-PEG2-kk- | 802 |
| | | | NH2 | |
| 127 | KLLIDV | Probe #127 | (FAM)-GKLLIDVGK(CPQ2)-NH2 | 803 |
| 128 | E(Nle)GVFD | Probe #128 | (FAM)-GE(Nle)GVFDGK(CPQ2)-PEG2-k- | 804 |
| | | | NH2 | |
| 129 | HQAYTL | Probe #129 | (FAM)-GHQAYTLGK(CPQ2)-PEG2-kk-NH2 | 805 |
| 130 | YVRKIQ | Probe #130 | (FAM)-GYVRKIQGK(CPQ2)-PEG2-k-NH2 | 806 |
| 131 | DRENSP | Probe #131 | (FAM)-GDRENSPGK(CPQ2)-NH2 | 807 |
| 132 | KYDKPR | Probe #132 | (FAM)-GKYDKPRGK(CPQ2)-NH2 | 808 |
| 133 | RPWKQL | Probe #133 | (FAM)-GRPWKQLGK(CPQ2)-PEG2-k-NH2 | 809 |
| 134 | APLQRY | Probe #134 | (FAM)-GAPLQRYGK(CPQ2)-NH2 | 810 |
| 135 | YQGQK(Nle) | Probe #135 | (FAM)-GYQGQK(Nle)GK(CPQ2)-NH2 | 811 |
| 136 | GRISSI | Probe #136 | (FAM)-GGRISSIGK(CPQ2)-NH2 | 812 |
| 137 | HSLTNV | Probe #137 | (FAM)-GHSLTNVGK(CPQ2)-PEG2-kk-NH2 | 813 |
| 138 | EWDFPE | Probe #138 | (FAM)-GEWDFPEGK(CPQ2)-PEG2-k-NH2 | 814 |
| 139 | YLA(Nle)DG | Probe #139 | (FAM)-GYLA(Nle)DGGK(CPQ2)-PEG2-k- | 815 |
| | | | NH2 | |
| 140 | FIY(Nle)PT | Probe #140 | (FAM)-GFIY(Nle)PTGK(CPQ2)-PEG2-k-NH2 | 816 |
| 141 | GHETWV | Probe #141 | (FAM)-GGHETWVGK(CPQ2)-PEG2-kk-NH2 | 817 |
| 142 | DYIGDE | Probe #142 | (FAM)-GDYIGDEGK(CPQ2)-PEG2-k-NH2 | 818 |
| 143 | AGTAHP | Probe #143 | (FAM)-GAGTAHPGK(CPQ2)-PEG2-kk-NH2 | 819 |
| 144 | V(Nle)TEIW | Probe #144 | (FAM)-GV(Nle)TEIWGK(CPQ2)-PEG2-k- | 820 |
| | | | NH2 | |
| 145 | PDDWQN | Probe #145 | (FAM)-GPDDWQNGK(CPQ2)-PEG2-k-NH2 | 821 |
| 146 | GLNQEY | Probe #146 | (FAM)-GGLNQEYGK(CPQ2)-PEG2-k-NH2 | 822 |
| 147 | YRDAVA | Probe #147 | (FAM)-GYRDAVAGK(CPQ2)-NH2 | 823 |
| 148 | TGPKGN | Probe #148 | (FAM)-GTGPKGNGK(CPQ2)-NH2 | 824 |
| 149 | DHVPQI | Probe #149 | (FAM)-GDHVPQIGK(CPQ2)-PEG2-kk-NH2 | 825 |
| 150 | NKEPIL | Probe #150 | (FAM)-GNKEPILGK(CPQ2)-NH2 | 826 |
| 151 | VWN(Nle)VH | Probe #151 | (FAM)-GVWN(Nle)VHGK(CPQ2)-PEG2-kk- | 827 |
| | | | NH2 | |
| 152 | PVIIEH | Probe #152 | (FAM)-GPVIIEHGK(CPQ2)-PEG2-kk-NH2 | 828 |
| 153 | FQTDNL | Probe #153 | (FAM)-GFQTDNLGK(CPQ2)-PEG2-k-NH2 | 829 |
| 154 | RF(Nle)HGI | Probe #154 | (FAM)-GRF(Nle)HGIGK(CPQ2)-PEG2-k- | 830 |
| | | | NH2 | |
| 155 | YAERTT | Probe #155 | (FAM)-GYAERTTGK(CPQ2)-NH2 | 831 |
| 156 | NRGELP | Probe #156 | (FAM)-GNRGELPGK(CPQ2)-NH2 | 832 |
| 157 | HHYFNY | Probe #157 | (FAM)-GHHYFNYGK(CPQ2)-PEG2-k-NH2 | 833 |
| 158 | STPYYH | Probe #158 | (FAM)-GSTPYYHGK(CPQ2)-PEG2-kk-NH2 | 834 |
| 159 | WFYPSA | Probe #159 | (FAM)-GWFYPSAGK(CPQ2)-PEG2-k-NH2 | 835 |
| 160 | SEFLFS | Probe #160 | (FAM)-GSEFLFSGK(CPQ2)-PEG2-k-NH2 | 836 |
| 161 | WYKTQY | Probe #161 | (FAM)-GWYKTQYGK(CPQ2)-NH2 | 837 |
| 162 | VTHLKV | Probe #162 | (FAM)-GVTHLKVGK(CPQ2)-PEG2-k-NH2 | 838 |
| 163 | INGGFS | Probe #163 | (FAM)-GINGGFSGK(CPQ2)-PEG2-k-NH2 | 839 |
| 164 | TVLGLD | Probe #164 | (FAM)-GTVLGLDGK(CPQ2)-PEG2-k-NH2 | 840 |
| 165 | SYWP(Nle)Q | Probe #165 | (FAM)-GSYWP(Nle)QGK(CPQ2)-PEG2-k- | 841 |
| | | | NH2 | |
| 166 | ASQQHR | Probe #166 | (FAM)-GASQQHRGK(CPQ2)-PEG2-k-NH2 | 842 |
| 167 | KNPAKA | Probe #167 | (FAM)-GKNPAKAGK(CPQ2)-PEG2-k-NH2 | 843 |
| 168 | (Nle)YWLVE | Probe #168 | (FAM)-G(Nle)YWLVEGK(CPQ2)-PEG2-k- | 844 |
| | | | NH2 | |
| 169 | SWWIFE | Probe #169 | (FAM)-GSWWIFEGK(CPQ2)-PEG2-k-NH2 | 845 |
| 170 | VNYEQD | Probe #170 | (FAM)-GVNYEQDGK(CPQ2)-PEG2-k-NH2 | 846 |
| 171 | HFF(Nle)AE | Probe #171 | (FAM)-GHFF(Nle)AEGK(CPQ2)-PEG2-kk- | 847 |
| | | | NH2 | |
| 172 | DIPPHW | Probe #172 | (FAM)-GDIPPHWGK(CPQ2)-PEG2-kk-NH2 | 848 |
| 173 | VDQW(Nle)W | Probe #173 | (FAM)-GVDQW(Nle)WGK(CPQ2)-PEG2-k- | 849 |
| | | | NH2 | |
| 174 | LRSL(Nle)K | Probe #174 | (FAM)-GLRSL(Nle)KGK(CPQ2)-PEG2-k- | 850 |
| | | | NH2 | |
| 175 | (Nle)(Nle) | Probe #175 | (FAM)-G(Nle)(Nle)IRHAGK(CPQ2)-PEG2-k- | 851 |
| | IRHA | | NH2 | |
| 176 | HDVKFI | Probe #176 | (FAM)-GHDVKFIGK(CPQ2)-PEG2-kk-NH2 | 852 |
| 177 | KRVQFL | Probe #177 | (FAM)-GKRVQFLGK(CPQ2)-PEG2-k-NH2 | 853 |
| 178 | RD(Nle) YAE | Probe #178 | (FAM)-GRD(Nle)YAEGK(CPQ2)-NH2 | 854 |
| 179 | L(Nle)IYFE | Probe #179 | (FAM)-GL(Nle)IYFEGK(CPQ2)-PEG2-k-NH2 | 855 |
| 180 | LRTKQS | Probe #180 | (FAM)-GLRTKQSGK(CPQ2)-PEG2-k-NH2 | 856 |
| 181 | WHGQQY | Probe #181 | (FAM)-GWHGQQYGK(CPQ2)-PEG2-kk- | 857 |
| | | | NH2 | |
| 182 | GPEGTI | Probe #182 | (FAM)-GGPEGTIGK(CPQ2)-PEG2-k-NH2 | 858 |
| 183 | ELDPIP | Probe #183 | (FAM)-GELDPIPGK(CPQ2)-PEG2-k-NH2 | 859 |
| 184 | GRAADF | Probe #184 | (FAM)-GGRAADFGK(CPQ2)-NH2 | 860 |
| 185 | HFIDYI | Probe #185 | (FAM)-GHFIDYIGK(CPQ2)-PEG2-kk-NH2 | 861 |
| 186 | S(Nle)(Nle) | Probe #186 | (FAM)-GS(Nle)(Nle)RVHGK(CPQ2)-PEG2-k- | 862 |
| | RVH | | NH2 | |
| 187 | SFRKII | Probe #187 | (FAM)-GSFRKIIGK(CPQ2)-PEG2-k-NH2 | 863 |
| 188 | TYE(Nle)FS | Probe #188 | (FAM)-GTYE(Nle)FSGK(CPQ2)-PEG2-k- | 864 |
| | | | NH2 | |
| 189 | HLLGFY | Probe #189 | (FAM)-GHLLGFYGK(CPQ2)-PEG2-kk-NH2 | 865 |
| 190 | (Nle)WTALT | Probe #190 | (FAM)-G(Nle)WTALTGK(CPQ2)-PEG2-k- | 866 |
| | | | NH2 | |
| 191 | IWN(Nle)VY | Probe #191 | (FAM)-GIWN(Nle)VYGK(CPQ2)-PEG2-k- | 867 |
| | | | NH2 | |
| 192 | RRNPLW | Probe #192 | (FAM)-GRRNPLWGK(CPQ2)-PEG2-k-NH2 | 868 |
| 193 | RWYGGI | Probe #193 | (FAM)-GRWYGGIGK(CPQ2)-NH2 | 869 |
| 194 | KTGDAR | Probe #194 | (FAM)-GKTGDARGK(CPQ2)-PEG2-k-NH2 | 870 |
| 195 | NYWEAN | Probe #195 | (FAM)-GNYWEANGK(CPQ2)-PEG2-k-NH2 | 871 |
| 196 | (Nle)QFDTS | Probe #196 | (FAM)-G(Nle)QFDTSGK(CPQ2)-PEG2-k- | 872 |
| | | | NH2 | |
| 197 | KRGAVE | Probe #197 | (FAM)-GKRGAVEGK(CPQ2)-PEG2-k-NH2 | 873 |
| 198 | SLKPTE | Probe #198 | (FAM)-GSLKPTEGK(CPQ2)-NH2 | 874 |
| 199 | ENDRLP | Probe #199 | (FAM)-GENDRLPGK(CPQ2)-NH2 | 875 |
| 200 | NSYQVQ | Probe #200 | (FAM)-GNSYQVQGK(CPQ2)-PEG2-k-NH2 | 876 |
| 201 | YPKEYL | Probe #201 | (FAM)-GYPKEYLGK(CPQ2)-NH2 | 877 |
| 202 | INNKWQ | Probe #202 | (FAM)-GINNKWQGK(CPQ2)-NH2 | 878 |
| 203 | (Nle)EFQGW | Probe #203 | (FAM)-G(Nle)EFQGWGK(CPQ2)-PEG2-k- | 879 |
| | | | NH2 | |
| 204 | PVRSTN | Probe #204 | (FAM)-GPVRSTNGK(CPQ2)-NH2 | 880 |
| 205 | SQAIKV | Probe #205 | (FAM)-GSQAIKVGK(CPQ2)-NH2 | 881 |
| 206 | WA(Nle)LYH | Probe #206 | (FAM)-GWA(Nle)LYHGK(CPQ2)-PEG2-kk- | 882 |
| | | | NH2 | |
| 207 | ISWIHA | Probe #207 | (FAM)-GISWIHAGK(CPQ2)-PEG2-kk-NH2 | 883 |
| 208 | AHDIV | Probe #208 | (FAM)-GAHDIVNGK(CPQ2)-PEG2-kk-NH2 | 884 |
| 209 | RHNVAS | Probe #209 | (FAM)-GRHNVASGK(CPQ2)-PEG2-k-NH2 | 885 |
| 210 | SVFVIE | Probe #210 | (FAM)-GSVFVIEGK(CPQ2)-PEG2-k-NH2 | 886 |
| 211 | FAKYYK | Probe #211 | (FAM)-GFAKYYKGK(CPQ2)-PEG2-k-NH2 | 887 |
| 212 | PYNTLQ | Probe #212 | (FAM)-GPYNTLQGK(CPQ2)-PEG2-k-NH2 | 888 |
| 213 | (Nle)DWGH | Probe #213 | (FAM)-G(Nle)DWGH(Nle)GK(CPQ2)-PEG2- | 889 |
| | (Nle) | | kk-NH2 | |
| 214 | SNREWF | Probe #214 | (FAM)-GSNREWFGK(CPQ2)-NH2 | 890 |
| 215 | GKSEHT | Probe #215 | (FAM)-GGKSEHTGK(CPQ2)-PEG2-kk-NH2 | 891 |
| 216 | FP(Nle)TDQ | Probe #216 | (FAM)-GFP(Nle)TDQGK(CPQ2)-PEG2-k- | 892 |
| | | | NH2 | |
| 217 | WSKFW(Nle) | Probe #217 | (FAM)-GWSKFW(Nle)GK(CPQ2) | 893 |
| 218 | RFTRPH | Probe #218 | (FAM)-GRFTRPHGK(CPQ2)-NH2 | 894 |
| 219 | QET(Nle)KD | Probe #219 | (FAM)-GQET(Nle)KDGK(CPQ2)-NH2 | 895 |
| 220 | HWWDVL | Probe #220 | (FAM)-GHWWDVLGK(CPQ2)-PEG2-kk- | 896 |
| | | | NH2 | |
| 221 | FNL V(Nle)S | Probe #221 | (FAM)-GFNLV(Nle)SGK(CPQ2)-PEG2-k- | 897 |
| | | | NH2 | |
| 222 | SAWRQR | Probe #222 | (FAM)-GSAWRQRGK(CPQ2)-PEG2-k-NH2 | 898 |
| 223 | TFHIFL | Probe #223 | (FAM)-GTFHIFLGK(CPQ2)-PEG2-kk-NH2 | 899 |
| 224 | WPQHVK | Probe #224 | (FAM)-GWPQHVKGK(CPQ2)-PEG2-k-NH2 | 900 |
| 225 | LI(Nle)HKN | Probe #225 | (FAM)-GLI(Nle)HKNGK(CPQ2)-PEG2-k- | 901 |
| | | | NH2 | |
| 226 | QDLEQP | Probe #226 | (FAM)-GQDLEQPGK(CPQ2)-PEG2-k-NH2 | 902 |
| 227 | HQKK(Nle)P | Probe #227 | (FAM)-GHQKK(Nle)PGK(CPQ2)-NH2 | 903 |
| 228 | GVTWLN | Probe #228 | (FAM)-GGVTWLNGK(CPQ2)-PEG2-k-NH2 | 904 |
| 229 | AGEPFK | Probe #229 | (FAM)-GAGEPFKGK(CPQ2)-NH2 | 905 |
| 230 | SR(Nle)ATT | Probe #230 | (FAM)-GSR(Nle)ATTGK(CPQ2)-NH2 | 906 |
| 231 | LAF(Nle)NH | Probe #231 | (FAM)-GLAF(Nle)NHGK(CPQ2)-PEG2-kk- | 907 |
| | | | NH2 | |
| 232 | PPSGLS | Probe #232 | (FAM)-GPPSGLSGK(CPQ2)-PEG2-k-NH2 | 908 |
| 233 | YTHSSP | Probe #233 | (FAM)-GYTHSSPGK(CPQ2)-PEG2-kk-NH2 | 909 |
| 234 | DGSHYR | Probe #234 | (FAM)-GDGSHYRGK(CPQ2)-PEG2-kk-NH2 | 910 |
| 235 | Y(Nle)GNGY | Probe #235 | (FAM)-GY(Nle)GNGYGK(CPQ2)-PEG2-k- | 911 |
| | | | NH2 | |
| 236 | DSITVS | Probe #236 | (FAM)-GDSITVSGK(CPQ2)-PEG2-k-NH2 | 912 |
| 237 | QTPNIQ | Probe #237 | (FAM)-GQTPNIQGK(CPQ2)-PEG2-k-NH2 | 913 |
| 238 | KLFFGY | Probe #238 | (FAM)-GKLFFGYGK(CPQ2)-NH2 | 914 |
| 239 | TQNFNW | Probe #239 | (FAM)-GTQNFNWGK(CPQ2)-PEG2-k-NH2 | 915 |
| 240 | YSDHEV | Probe #240 | (FAM)-GYSDHEVGK(CPQ2)-PEG2-kk-NH2 | 916 |
| 241 | RYVVPA | Probe #241 | (FAM)-GRYVVPAGK(CPQ2)-NH2 | 917 |
| 242 | ILHRIR | Probe #242 | (FAM)-GILHRIRGK(CPQ2)-NH2 | 918 |
| 243 | ESDNQ(Nle) | Probe #243 | (FAM)-GESDNQ(Nle)GK(CPQ2)-PEG2-k- | 919 |
| | | | NH2 | |
| 244 | YDDKG(Nle) | Probe #244 | (FAM)-GYDDKG(Nle)GK(CPQ2)-NH2 | 920 |
| 245 | QLS(Nle)VW | Probe #245 | (FAM)-GQLS(Nle)VWGK(CPQ2)-PEG2-k- | 921 |
| | | | NH2 | |
| 246 | PGGER(Nle) | Probe #246 | (FAM)-GPGGER(Nle)GK(CPQ2)-NH2 | 922 |
| 247 | WKHHPD | Probe #247 | (FAM)-GWKHHPDGK(CPQ2)-NH2 | 923 |
| 248 | QWVDED | Probe #248 | (FAM)-GQWVDEDGK(CPQ2)-PEG2-k-NH2 | 924 |
| 249 | NAYNEI | Probe #249 | (FAM)-GNAYNEIGK(CPQ2)-PEG2-k-NH2 | 925 |
| 250 | EEKAPR | Probe #250 | (FAM)-GEEKAPRGK(CPQ2)-PEG2-kk-NH2 | 926 |
| 251 | PWQIGK | Probe #251 | (FAM)-GPWQIGKGK(CPQ2)-NH2 | 927 |
| 252 | IAQVGN | Probe #252 | (FAM)-GIAQVGNGK(CPQ2)-PEG2-k-NH2 | 928 |
| 253 | V(Nle)RQSE | Probe #253 | (FAM)-GV(Nle)RQSEGK(CPQ2)-NH2 | 929 |
| 254 | TERVDA | Probe #254 | (FAM)-GTERVDAGK(CPQ2)-NH2 | 930 |
| 255 | WLRWRL | Probe #255 | (FAM)-GWLRWRLGK(CPQ2)-PEG2-k-NH2 | 931 |
| 256 | WKTKGQ | Probe #256 | (FAM)-GWKTKGQGK(CPQ2)-PEG2-k-NH2 | 932 |
| 257 | QSNGDV | Probe #257 | (FAM)-GQSNGDVGK(CPQ2)-PEG2-k-NH2 | 933 |
| 258 | TLFYAL | Probe #258 | (FAM)-GTLFYALGK(CPQ2)-PEG2-k-NH2 | 934 |
| 259 | TVTLNP | Probe #259 | (FAM)-GTVTLNPGK(CPQ2)-PEG2-k-NH2 | 935 |
| 260 | YAFGRK | Probe #260 | (FAM)-GYAFGRKGK(CPQ2)-PEG2-k-NH2 | 936 |
| 261 | DYNYWD | Probe #261 | (FAM)-GDYNYWDGK(CPQ2)-PEG2-k-NH2 | 937 |
| 262 | EWHEII | Probe #262 | (FAM)-GEWHEIIGK(CPQ2)-PEG2-kk-NH2 | 938 |
| 263 | QKAAWD | Probe #263 | (FAM)-GQKAAWDGK(CPQ2)-NH2 | 939 |
| 264 | DNTSAD | Probe #264 | (FAM)-GDNTSADGK(CPQ2)-PEG2-k-NH2 | 940 |
| 265 | HEGEYV | Probe #265 | (FAM)-GHEGEYVGK(CPQ2)-PEG2-kk-NH2 | 941 |
| 266 | WSPSFK | Probe #266 | (FAM)-GWSPSFKGK(CPQ2)-NH2 | 942 |
| 267 | HDEHWT | Probe #267 | (FAM)-GHDEHWTGK(CPQ2)-PEG2-kk-NH2 | 943 |
| 268 | YVW(Nle)RD | Probe #268 | (FAM)-GYVW(Nle)RDGK(CPQ2)-NH2 | 944 |
| 269 | (Nle)DP(Nle)KF | Probe #269 | (FAM)-G(Nle)DP(Nle)KFGK(CPQ2)-NH2 | 945 |
| 270 | (Nle)R(Nle) | Probe #270 | (FAM)-G(Nle)R(Nle)FWDGK(CPQ2)-NH2 | 946 |
| | FWD | | | |
| 271 | DIAIT(Nle) | Probe #271 | (FAM)-GDIAIT(Nle)GK(CPQ2)-PEG2-k-NH2 | 947 |
| 272 | PI(Nle)RFH | Probe #272 | (FAM)-GPI(Nle)RFHGK(CPQ2)-PEG2-k-NH2 | 948 |
| 273 | VWQGYI | Probe #273 | (FAM)-GVWQGYIGK(CPQ2)-PEG2-k-NH2 | 949 |
| 274 | KK(Nle)SNP | Probe #274 | (FAM)-GKK(Nle)SNPGK(CPQ2)-PEG2-k- | 950 |
| | | | NH2 | |
| 275 | GHPLSP | Probe #275 | (FAM)-GGHPLSPGK(CPQ2)-PEG2-kk-NH2 | 951 |
| 276 | VRQHKP | Probe #276 | (FAM)-GVRQHKPGK(CPQ2)-NH2 | 952 |
| 277 | AQNFYR | Probe #277 | (FAM)-GAQNFYRGK(CPQ2)-NH2 | 953 |
| 278 | VAGKSI | Probe #278 | (FAM)-GVAGKSIGK(CPQ2)-NH2 | 954 |
| 279 | LVGQVN | Probe #279 | (FAM)-GLVGQVNGK(CPQ2)-PEG2-k-NH2 | 955 |
| 280 | QVKHFT | Probe #280 | (FAM)-GQVKHFTGK(CPQ2)-PEG2-k-NH2 | 956 |
| 28 | QKSVVS | Probe #281 | (FAM)-GQKSVVSGK(CPQ2)-NH2 | 957 |
| 282 | Y(Nle)QEWL | Probe #282 | (FAM)-GY(Nle)QEWLGK(CPQ2)-PEG2-k- | 958 |
| | | | NH2 | |
| 283 | G(Nle)YIDE | Probe #283 | (FAM)-GG(Nle)YIDEGK(CPQ2)-PEG2-k- | 959 |
| | | | NH2 | |
| 284 | NAGSKF | Probe #284 | (FAM)-GNAGSKFGK(CPQ2)-NH2 | 960 |
| 285 | EFVHNP | Probe #285 | (FAM)-GEFVHNPGK(CPQ2)-PEG2-kk-NH2 | 961 |
| 286 | WE(Nle) VKI | Probe #286 | (FAM)-GWE(Nle)VKIGK(CPQ2)-NH2 | 962 |
| 287 | WVGASH | Probe #287 | (FAM)-GWVGASHGK(CPQ2)-PEG2-kk-NH2 | 963 |
| 288 | ITTLY(Nle) | Probe #288 | (FAM)-GITTLY(Nle)GK(CPQ2)-PEG2-k- | 964 |
| | | | NH2 | |
| 289 | GHIDEY | Probe #289 | (FAM)-GGHIDEYGK(CPQ2)-PEG2-kk-NH2 | 965 |
| 290 | KV(Nle)DYG | Probe #290 | (FAM)-GKV(Nle)DYGGK(CPQ2)-NH2 | 966 |
| 29 | QEKQT(Nle) | Probe #291 | (FAM)-GQEKQT(Nle)GK(CPQ2)-NH2 | 967 |
| 292 | EVGHEA | Probe #292 | (FAM)-GEVGHEAGK(CPQ2)-PEG2-kk-NH2 | 968 |
| 293 | AWEGQY | Probe #293 | (FAM)-GAWEGQYGK(CPQ2)-PEG2-k-NH2 | 969 |
| 294 | FLVQWT | Probe #294 | (FAM)-GFLVQWTGK(CPQ2)-PEG2-k-NH2 | 970 |
| 295 | SKWGYW | Probe #295 | (FAM)-GSKWGYWGK(CPQ2)-NH2 | 971 |
| 296 | TWIS(Nle)Q | Probe #296 | (FAM)-GTWIS(Nle)QGK(CPQ2)-PEG2-k- | 972 |
| | | | NH2 | |
| 297 | VIDKDF | Probe #297 | (FAM)-GVIDKDFGK(CPQ2)-NH2 | 973 |
| 298 | VKFAIY | Probe #298 | (FAM)-GVKFAIYGK(CPQ2)-NH2 | 974 |
| 299 | HNQ(Nle)KS | Probe #299 | (FAM)-GHNQ(Nle)KSGK(CPQ2)-PEG2-k- | 975 |
| | | | NH2 | |
| 300 | QYVFF(Nle) | Probe #300 | (FAM)-GQYVFF(Nle)GK(CPQ2)-PEG2-k- | 976 |
| | | | NH2 | |
| 301 | YNPRE(Nle) | Probe #301 | (FAM)-GYNPRE(Nle)GK(CPQ2)-NH2 | 977 |
| 302 | KHG(Nle)PE | Probe #302 | (FAM)-GKHG(Nle)PEGK(CPQ2)-PEG2-kk- | 978 |
| | | | NH2 | |
| 303 | WSREYW | Probe #303 | (FAM)-GWSREYWGK(CPQ2)-NH2 | 979 |
| 304 | IDRVDK | Probe #304 | (FAM)-GIDRVDKGK(CPQ2)-PEG2-kk-NH2 | 980 |
| 305 | GDRENSPK(CP | Probe #305 | (FAM)-kkGDRENSPK(CPQ2)L-OH | 981 |
| | Q2)L-OH | | | |
| 306 | GDRENSPLK(C | Probe #306 | (FAM)-kkGDRENSPLK(CPQ2)-OH | 982 |
| | PQ2)-OH | | | |
| 307 | NAGSKFK(CPQ | Probe #307 | (FAM)-GNAGSKFK(CPQ2)Q-OH | 983 |
| | 2)Q-OH | | | |
| 308 | NAGSKFQK(CP | Probe #308 | (FAM)-GNAGSKFQK(CPQ2)-OH | 984 |
| | Q2)-OH | | | |
| 309 | GHLLGFYK(CP | Probe #309 | (FAM)-kkGHLLGFYK(CPQ2)V-OH | 985 |
| | Q2)V-OH | | | |
| 310 | GHLLGFYVK( | Probe #310 | FAM)-kkGHLLGFYVK(CPQ2)-OH | 986 |
| | CPQ2)-OH | | | |
| 311 | GQEKQT(Nle)K | Probe #311 | (FAM)-kkGQEKQT(Nle)K(CPQ2)(Nle)-OH | 987 |
| | (CPQ2)(Nle)-OH | | | |
| 312 | GQEKQT(Nle) | Probe #312 | (FAM)-kkGQEKQT(Nle)(Nle)K(CPQ2)-OH | 988 |
| | (Nle)K(CPQ2)- | | | |
| | OH | | | |
| 313 | kGDPFVVSK(C | Probe #313 | (FAM)-kGDPFVVSK(CPQ2)W-OH | 989 |
| | PQ2)W-OH | | | |
| 314 | kGDPFVVSWK | Probe #314 | (FAM)-kGDPFVVSWK(CPQ2)-OH | 990 |
| | (CPQ2)-OH | | | |
| 315 | NAYNEIK(CPQ | Probe #315 | (FAM)-GNAYNEIK(CPQ2)R-OH | 991 |
| | 2)R-OH | | | |
| 316 | NAYNEIRK(CP | Probe #316 | FAM)-GNAYNEIRK(CPQ2)-OH | 992 |
| | Q2)-OH | | | |
| 317 | V(Nle)RQSEK( | Probe #317 | (FAM)-GV(Nle)RQSEK(CPQ2)N-OH | 993 |
| | CPQ2)N-OH | | | |
| 318 | V(Nle)RQSENK | Probe #318 | (FAM)-GV(Nle)RQSENK(CPQ2) | 994 |
| | (CPQ2)-OH | | | |
| 319 | YNPRE(Nle)K | Probe #319 | (FAM)-GYNPRE(Nle)K(CPQ2)I-OH | 995 |
| | (CPQ2)I-OH | | | |
| 320 | YNPRE(Nle)IK | Probe #320 | (FAM)-GYNPRE(Nle)IK(CPQ2)-OH | 996 |
| | (CPQ2)-OH | | | |
| 321 | EFVHNPK(CPQ | Probe #321 | (FAM)-kGEFVHNPK(CPQ2)K-OH | 997 |
| | 2)K-OH | | | |
| 322 | EFVHNPKK(CP | Probe #322 | (FAM)-kGEFVHNPKK(CPQ2)-OH | 998 |
| | Q2)-OH | | | |
| 323 | KRVQFLK(CPQ | Probe #323 | (FAM)-GKRVQFLK(CPQ2)H-OH | 999 |
| | 2)H-OH | | | |
| 324 | KRVQFLHK(CP | Probe #324 | (FAM)-GKRVQFLHK(CPQ2)-OH | 1000 |
| | Q2)-OH | | | |
| 325 | LI(Nle)HKNK | Probe #325 | (FAM)-kGLI(Nle)HKNK(CPQ2)G-OH | 1001 |
| | (CPQ2)G-OH | | | |
| 326 | LI(Nle)HKNGK | Probe #326 | (FAM)-kGLI(Nle)HKNGK(CPQ2)-OH | 1002 |
| | (CPQ2)-OH | | | |
| 327 | WA(Nle)LYHK | Probe #327 | (FAM)-kkGWA(Nle)LYHK(CPQ2)S-OH | 1003 |
| | (CPQ2)S-OH | | | |
| 328 | WA(Nle)LYHS | Probe #328 | (FAM)-kkGWA(Nle)LYHSK(CPQ2)-OH | 1004 |
| | K(CPQ2)-OH | | | |
| 329 | AHDIVNK(CPQ | Probe #329 | (FAM)-kkGAHDIVNK(CPQ2)Y-OH | 1005 |
| | 2)Y-OH | | | |
| 330 | AHDIVNYK(CP | Probe #330 | (FAM)-kkGAHDIVNYK(CPQ2)-OH | 1006 |
| | Q2)-OH | | | |
| 331 | SVFVIEK | Probe #331 | (FAM)-kGSVFVIEK(CPQ2)P-OH | 1007 |
| | (CPQ2)P-OH | | | |
| 332 | SVFVIEPK | Probe #332 | (FAM)-kGSVFVIEPK(CPQ2)-OH | 1008 |
| | (CPQ2)-OH | | | |
| 333 | PPSGLSK | Probe #333 | (FAM)-kGPPSGLSK(CPQ2)E-OH | 1009 |
| | (CPQ2)E-OH | | | |
| 334 | PPSGLSEK | Probe #334 | (FAM)-kGPPSGLSEK(CPQ2)-OH | 1010 |
| | (CPQ2)-OH | | | |
| 335 | RWYGGIK(CP | Probe #335 | (FAM)-kkGRWYGGIK(CPQ2)F-OH | 1011 |
| | Q2)F-OH | | | |
| 336 | RWYGGIFK | Probe #336 | (FAM)-kkGRWYGGIFK(CPQ2)-OH | 1012 |
| | (CPQ2)-OH | | | |
| 337 | QYVFF(Nle)K | Probe #337 | (FAM)-kGQYVFF(Nle)K(CPQ2)D-OH | 1013 |
| | (CPQ2)D-OH | | | |
| 338 | QYVFF(Nle)DK | Probe #338 | (FAM)-kGQYVFF(Nle)DK(CPQ2)-OH | 1014 |
| | (CPQ2)-OH | | | |
| 339 | FAKYYKK(CP | Probe #339 | (FAM)-kGFAKYYKK(CPQ2)T-OH | 1015 |
| | Q2)T-OH | | | |
| 340 | FAKYYKTK(CP | Probe #340 | (FAM)-KGFAKYYKTK(CPQ2)-OH | 1016 |
| | Q2)-OH | | | |
| 341 | QVKHFTK(CPQ | Probe #341 | (FAM)-kGQVKHFTK(CPQ2)A-OH | 1017 |
| | 2)A-OH | | | |
| 342 | QVKHFTAK(CP | Probe #342 | (FAM)-kGQVKHFTAK(CPQ2)-OH | 1018 |
| | Q2)-OH | | | |
| | APK(CPQ2)-OH | Probe #343 | FAM-APK(CPQ2)-OH | |
| 344 | NH2- | Probe #344 | NH2-HK(FAM)DRENSPGK(CPQ2)-NH2 | 1019 |
| | HK(FAM)DREN | | | |
| | SP | | | |
| 345 | NH2- | Probe #345 | NH2-K(FAM)HDRENSPGK(CPQ2)-NH2 | 1020 |
| | K(FAM)HDREN | | | |
| | SP | | | |
| 346 | NH2- | Probe #346 | NH2-WK(FAM)NAGSKFGKK(CPQ2)-NH2 | 1021 |
| | WK(FAM)NAG | | | |
| | SKF | | | |
| 347 | NH2- | Probe #347 | NH2-K(FAM)WNAGSKFGKK(CPQ2)-NH2 | 1022 |
| | K(FAM)WNAG | | | |
| | SKF | | | |
| 348 | NH2- | Probe #348 | NH2-SK(FAM)HLLGFYGKK(CPQ2)-NH2 | 1023 |
| | SK(FAM)HLLG | | | |
| | FY | | | |
| 349 | NH2- | Probe #349 | NH2-K(FAM)SHLLGFYGKK(CPQ2)-NH2 | 1024 |
| | K(FAM)SHLLG | | | |
| | FY | | | |
| 350 | NH2- | Probe #350 | NH2-KK(FAM)QEKQT(Nle)GK(CPQ2)-NH2 | 1025 |
| | KK(FAM)QEKQ | | | |
| | T(Nle) | | | |
| 351 | NH2- | Probe #351 | NH2-K(FAM)KQEKQT(Nle)GK(CPQ2)-NH2 | 1026 |
| | K(FAM)KQEKQ | | | |
| | T(Nle) | | | |
| 352 | NH2- | Probe #352 | NH2-GK(FAM)DPFVVSGK(CPQ2)-NH2 | 1027 |
| | GK(FAM)DPFV | | | |
| | VS | | | |
| 353 | NH2- | Probe #353 | NH2-K(FAM)GDPFVVSGK(CPQ2)-NH2 | 1028 |
| | K(FAM)GDPFV | | | |
| | VS | | | |
| 354 | NH2- | Probe #354 | NH2-PK(FAM)NAYNEIGK(CPQ2)-NH2 | 1029 |
| | PK(FAM)NAYN | | | |
| | EI | | | |
| 355 | NH2- | Probe #355 | NH2-K(FAM)PNAYNEIGK(CPQ2)-NH2 | 1030 |
| | K(FAM)PNAYN | | | |
| | EI | | | |
| 356 | NH2- | Probe #356 | NH2-DK(FAM)V(Nle)RQSEGkK(CPQ2)- | 1031 |
| | DK(FAM)V(Nle) | | NH2 | |
| | RQSE | | | |
| 357 | NH2- | Probe #357 | NH2-K(FAM)DV(Nle)RQSEGKK(CPQ2)- | 1032 |
| | K(FAM)DV | | NH2 | |
| | (Nle)RQSE | | | |
| 358 | NH2- | Probe #358 | NH2-EK(FAM)YNPRE(Nle)GkK(CPQ2)-NH2 | 1033 |
| | EK(FAM)YNPR | | | |
| | E(Nle) | | | |
| 359 | NH2- | Probe #359 | NH2-K(FAM)EYNPRE(Nle)GkK(CPQ2)-NH2 | 1034 |
| | K(FAM)EYNPR | | | |
| | E(Nle) | | | |
| 360 | NH2- | Probe #360 | NH2-TK(FAM)EFVHNPGkK(CPQ2)-NH2 | 1035 |
| | TK(FAM)EFVH | | | |
| | NP | | | |
| 361 | NH2- | Probe #361 | NH2-K(FAM)TEFVHNPGkK(CPQ2)-NH2 | 1036 |
| | K(FAM)TEFVH | | | |
| | NP | | | |
| 362 | NH2- | Probe #362 | NH2-QK(FAM)KRVQFLGK(CPQ2)-NH2 | 1037 |
| | QK(FAM)KRV | | | |
| | QFL | | | |
| 363 | NH2- | Probe #363 | NH2-K(FAM)QKRVQFLGK(CPQ2)-NH2 | 1038 |
| | K(FAM)QKRV | | | |
| | QFL | | | |
| 364 | NH2- | Probe #364 | NH2-YK(FAM)LI(Nle)HKNGK(CPQ2)-NH2 | 1039 |
| | YK(FAM)LI | | | |
| | (Nle)HKN | | | |
| 365 | NH2- | Probe #365 | NH2-K(FAM)YLI(Nle)HKNGK(CPQ2)-NH2 | 1040 |
| | K(FAM)YLI | | | |
| | (Nle)HKN | | | |
| 366 | NH2- | Probe #366 | NH2-FK(FAM)WA(Nle)LYHGkK(CPQ2)- | 1041 |
| | FK(FAM)WA | | NH2 | |
| | (Nle)LYH | | | |
| 367 | NH2- | Probe #367 | NH2-K(FAM)FWA(Nle)LYHGkK(CPQ2)- | 1042 |
| | K(FAM)FWA(N | | NH2 | |
| | le)LYH | | | |
| 368 | NH2- | Probe #368 | NH2-IK(FAM)AHDIVNGKK(CPQ2)-NH2 | 1043 |
| | IK(FAM)AHDI | | | |
| | VN | | | |
| 369 | NH2- | Probe #369 | NH2-K(FAM)IAHDIVNGkK(CPQ2)-NH2 | 1044 |
| | K(FAM)IAHDI | | | |
| | VN | | | |
| 370 | NH2- | Probe #370 | NH2-VK(FAM)SVFVIEGK(CPQ2)-NH2 | 1045 |
| | VK(FAM)SVFV | | | |
| | IE | | | |
| 371 | NH2- | Probe #371 | NH2-K(FAM)VSVFVIEGK(CPQ2)-NH2 | 1046 |
| | K(FAM)VSVFV | | | |
| | IE | | | |
| 372 | NH2- | Probe #372 | NH2-(Nle)K(FAM)PPSGLSGK(CPQ2)-NH2 | 1047 |
| | (Nle)K(FAM)PP | | | |
| | SGLS | | | |
| 373 | NH2- | Probe #373 | NH2-K(FAM)(Nle)PPSGLSGK(CPQ2)-NH2 | 1048 |
| | K(FAM)(Nle)PP | | | |
| | SGLS | | | |
| 374 | NH2- | Probe #374 | NH2-LK(FAM)RWYGGIGKK(CPQ2)-NH2 | 1049 |
| | LK(FAM)RWY | | | |
| | GGI | | | |
| 375 | NH2- | Probe #375 | NH2-K(FAM)LRWYGGIGKK(CPQ2)-NH2 | 1050 |
| | K(FAM)LRWY | | | |
| | GGI | | | |
| 376 | NH2- | Probe #376 | NH2-NK(FAM)QYVFF(Nle)GK(CPQ2)-NH2 | 1051 |
| | NK(FAM)QYVF | | | |
| | F(Nle) | | | |
| 377 | NH2- | Probe #377 | NH2-K(FAM)NQYVFF(Nle)GK(CPQ2)-NH2 | 1052 |
| | K(FAM)NQYVF | | | |
| | F(Nle) | | | |
| 378 | NH2- | Probe #378 | NH2-AK(FAM)FAKYYKGK(CPQ2)-NH2 | 1053 |
| | AK(FAM)FAKY | | | |
| | YK | | | |
| 379 | NH2- | Probe #379 | NH2-K(FAM)AFAKYYKGK(CPQ2)-NH2 | 1054 |
| | K(FAM)AFAKY | | | |
| | YK | | | |
| 380 | NH2- | Probe #380 | NH2-RK(FAM)QVKHFTGK(CPQ2)-NH2 | 1055 |
| | RK(FAM)QVK | | | |
| | HFT | | | |
| 381 | NH2- | Probe #381 | NH2-K(FAM)RQVKHFTGK(CPQ2)-NH2 | 1056 |
| | K(FAM)RQVK | | | |
| | HFT | | | |
| | NH2-K(FAM)PP | Probe #382 | NH2-K(FAM)PPK(CPQ2)-NH2 | 1057 |
| 383 | kpilffrlk | Probe #383 | 5FAM-GkpilffrlkGK(CPQ2)-PEG2-kk-NH2 | 1058 |
| | LRR | Probe #384 | Boc-Leu-Arg-Arg-AMC | |
| | R | Probe #385 | Arg-AMC | |
| | VR | Probe #386 | Boc-Val-Arg-AMC | |
| | RR | Probe #387 | Z-Arg-Arg-AMC | |
| | GR | Probe #388 | Gly-Arg-AMC | |
| | FR | Probe #389 | Z-Phe-Arg-AMC | |
| | RGK | Probe #390 | Ac-Arg-Gly-Lys-AMC | |
| | GGR | Probe #391 | Z-Gly-Gly-Arg-AMC | |
| | F | Probe #392 | Glutaryl-Phe-AMC | |
| | D | Probe #393 | H-Asp-AMC | |
| | RR | Probe #394 | H-Arg-Arg-AMC | |
| | R | Probe #395 | Z-Arg-AMC | |
| | Bz-R | Probe #396 | Bz-Arg-AMC | |
| | Bz-R | Probe #397 | Bz-Arg-AMC | |
| | PR | Probe #398 | Z-Pro-Arg-AMC | |
| | GPR | Probe #399 | Z-Gly-Pro-Arg-AMC | |
| | LR | Probe #400 | Z-Leu-Arg-AMC | |
| | PFR | Probe #401 | H-Pro-Phe-Arg-AMC | |
| | LLR | Probe #402 | Z-Leu-Leu-Arg-AMC | |
| | QRR | Probe #403 | Boc-Gln-Arg-Arg-AMC | |
| | GR | Probe #404 | Glutaryl-Gly-Arg-AMC | |
| | GRR | Probe #405 | Boc-Gly-Arg-Arg-AMC | |
| 406 | LRGG | Probe #406 | Z-Leu-Arg-Gly-Gly-AMC | 1059 |
| 407 | RLRGG | Probe #407 | 5-FAM-GRLRGGGK(CPQ2)-PEG2-kk-GC | 1060 |
| | | | | and |
| | | | | 1297 |
| 408 | RELNGGAPI | Probe #408 | 5-FAM-GRELNGGAPIGK(CPQ2)-PEG2-kk- | 1061 |
| | | | GC | and |
| | | | | 1297 |
| 409 | TSAVLQSGFR | Probe #409 | 5-FAM-GTSAVLQSGFRKGK(CPQ2)-PEG2- | 1062 |
| | K | | kk-GC | and |
| | | | | 1297 |
| 410 | SGVTFQGKFK | Probe #410 | 5-FAM-GSGVTFQGKFKKGK(CPQ2)-PEG2- | 1063 |
| | K | | kk-GC | and |
| | | | | 1297 |
| 411 | AAFA | Probe #411 | 5-FAM-GAAFAGK(CPQ2)-PEG2-kk-GC | 1064 |
| | | | | and |
| | | | | 1297 |
| 412 | HGDQMAQKS | Probe #412 | 5FAM-GHGDQMAQKS-K(CPQ2)-PEG2- | 1065 |
| | | | DLys-DLys-GC-NH2 | and |
| | | | | 1298 |
| 413 | GPLGMR | Probe #413 | 5FAM-GGPLGMRG-K(CPQ2)-PEG2-DLys- | 1066 |
| | | | DLys-GC-NH2 | and |
| | | | | 1298 |
| 414 | FFLAQA- | Probe #414 | 5FAM-GFFLAQA-HomoPhe-RSK-K(CPQ2)- | 1067 |
| | HomoPhe-RSK | | PEG2-DLys-DLys-GC-NH2 | and |
| | | | | 1298 |
| 415 | AHAVSRIRIYL | Probe #415 | 5FAM-GAHAVSRIRIYLLPAK-K(CPQ2)- | 1068 |
| | LPAK | | PEG2-DLys-DLys-GC-NH2 | and |
| | | | | 1298 |
| 416 | PLALWAR | Probe #416 | 5FAM-GPLALWAR-K(CPQ2)-PEG2-DLys- | 1069 |
| | | | DLys-GC-NH2 | and |
| | | | | 1298 |
| 417 | PLA- | Probe #417 | 5FAM-GPLA-C(OMeBzl)-WAR-K(CPQ2)- | 1070 |
| | C(OMeBzl)- | | PEG2-DLys-DLys-GC-NH2 | and |
| | WAR | | | 1298 |
| 418 | APRWIQD | Probe #418 | 5FAM-GAPRWIQD-K(CPQ2)-PEG2-DLys- | 1071 |
| | | | DLys-GC-NH2 | and |
| | | | | 1298 |
| 419 | LREQQRLKS | Probe #419 | 5FAM-GLREQQRLKS-K(CPQ2)-PEG2- | 1072 |
| | | | DLys-DLys-GC-NH2 | and |
| | | | | 1298 |
| 420 | EFPIYVFLPAK | Probe #420 | 5FAM-GEFPIYVFLPAKK-K(CPQ2)-PEG2- | 1073 |
| | K | | DLys-DLys-GC-NH2 | and |
| | | | | 1298 |
| 421 | GAANLVRGG | Probe #421 | 5FAM-GGAANLVRGG-K(CPQ2)-PEG2- | 1074 |
| | | | DLys-DLys-GC-NH2 | and |
| | | | | 1298 |
| 422 | GYAELRMG | Probe #422 | 5FAM-GGYAELRMGG-K(CPQ2)-PEG2- | 1075 |
| | | | DLys-DLys-GC-NH2 | and |
| | | | | 1298 |
| 423 | AAGAMFLEA | Probe #423 | 5FAM-GAAGAMFLEA-K(CPQ2)-PEG2- | 1076 |
| | | | DLys-DLys-GC-NH2 | and |
| | | | | 1298 |
| 424 | LGGSGQRGRK | Probe #424 | (FAM)-GLGGSGQRGRKALEG-K(CPQ2)- | 1077 |
| | ALE | | (PEG2)-DLys-DLys-GC | and |
| | | | | 1297 |
| 425 | LGGSGHYGRS | Probe #425 | (FAM)-GLGGSGHYGRSGLEG-K(CPQ2)- | 1078 |
| | | | (PEG2)-DLys-DLys-GC | and |
| | GLE | | | 1297 |
| 426 | YGRS | Probe #426 | (FAM)-GYGRSG-K(CPQ2)-(PEG2)-DLys- | 1079 |
| | | | DLys-GC | and |
| | | | | 1297 |
| 427 | FRGRK | Probe #427 | (FAM)-GFRGRKG-K(CPQ2)-(PEG2)-DLys- | 1080 |
| | | | DLys-GC | and |
| | | | | 1297 |
| 428 | DRRKKLTQ | Probe #428 | (FAM)-GDRRKKLTQG-K(CPQ2)-(PEG2)- | 1081 |
| | | | DLys-DLys-GC | and |
| | | | | 1297 |
| 429 | HPGGPQ | Probe #429 | (FAM)-GHPGGPQG-K(CPQ2)-(PEG2)-DLys- | 1082 |
| | | | DLys-GC | and |
| | | | | 1297 |
| 430 | KLRFSKQ | Probe #430 | (FAM)-GKLRFSKQG-K(CPQ2)-(PEG2)- | 1083 |
| | | | DLys-DLys-GC | and |
| | | | | 1297 |
| 431 | AIKFFSAQ | Probe #431 | (FAM)-GAIKFFSAQG-K(CPQ2)-(PEG2)- | 1084 |
| | | | DLys-DLys-GC | and |
| | | | | 1297 |
| 432 | AIKFFVRQ | Probe #432 | (FAM)-GAIKFFVRQG-K(CPQ2)-(PEG2)- | 1085 |
| | | | DLys-DLys-GC | and |
| | | | | 1297 |
| 433 | RPPGFSAFK | Probe #433 | (FAM)-GRPPGFSAFKG-K(CPQ2)-(PEG2)- | 1086 |
| | | | DLys-DLys-GC | and |
| | | | | 1297 |
| 434 | FAP-QLS | Probe #434 | (FAM)-GFAP-QLSG-K(CPQ2)-(PEG2)-DLys- | 1087 |
| | | | DLys-GC | and |
| | | | | 1297 |
| 435 | FAA-QMA | Probe #435 | (FAM)-GFAA-QMAG-K(CPQ2)-(PEG2)- | 1088 |
| | | | DLys-DLys-GC | and |
| | | | | 1297 |
| 436 | GMP-ANQ | Probe #436 | (FAM)-GGMP-ANQG-K(CPQ2)-(PEG2)- | 1089 |
| | | | DLys-DLys-GC | and |
| | | | | 1297 |
| 437 | LSGRSDNH | Probe #437 | (FAM)-GLSGRSDNHG-K(CPQ2)-(PEG2)- | 1090 |
| | | | DLys-DLys-GC | and |
| | | | | 1297 |
| 438 | MAALITRPDF | Probe #438 | (FAM)-GMAALITRPDFG-K(CPQ2)-(PEG2)- | 1091 |
| | | | DLys-DLys-GC | and |
| | | | | 1297 |
| 439 | MAAAITRPRF | Probe #439 | (FAM)-GMAAAITRPRFG-K(CPQ2)-(PEG2)- | 1092 |
| | | | DLys-DLys-GC | and |
| | | | | 1297 |
| 440 | MAALIVRPDL | Probe #440 | (FAM)-GMAALIVRPDLG-K(CPQ2)-(PEG2)- | 1093 |
| | | | DLys-DLys-GC | and |
| | | | | 1297 |
| 441 | TSGPNQEQE | Probe #441 | (FAM)-GTSGPNQEQEG-K(CPQ2)-(PEG2)- | 1094 |
| | | | DLys-DLys-GC | and |
| | | | | 1297 |
| 442 | TAGPNQEQE | Probe #442 | (FAM)-GTAGPNQEQEG-K(CPQ2)-(PEG2)- | 1095 |
| | | | DLys-DLys-GC | and |
| | | | | 1297 |
| 443 | GPGPNQA | Probe #443 | (FAM)-GGPGPNQAG-K(CPQ2)-(PEG2)- | 1096 |
| | | | DLys-DLys-GC | and |
| | | | | 1297 |
| 444 | ASGPAGPA | Probe #444 | (FAM)-GASGPAGPAG-K(CPQ2)-(PEG2)- | 1097 |
| | | | DLys-DLys-GC | and |
| | | | | 1297 |
| 445 | ERGETGPSG | Probe #445 | (FAM)-GERGETGPSGG-K(CPQ2)-(PEG2)- | 1098 |
| | | | DLys-DLys-GC | and |
| | | | | 1297 |
| 446 | VSQELGQR | Probe #446 | (FAM)-GVSQELGQRG-K(CPQ2)-(PEG2)- | 1099 |
| | | | DLys-DLys-GC | and |
| | | | | 1297 |
| 447 | TGPPGYPTG | Probe #447 | (FAM)-GTGPPGYPTGG-K(CPQ2)-(PEG2)- | 1100 |
| | | | DLys-DLys-GC | and |
| | | | | 1297 |
| 448 | TRLPVYQ | Probe #448 | (FAM)-GTRLPVYQG-K(CPQ2)-(PEG2)- | 1101 |
| | | | DLys-DLys-GC | and |
| | | | | 1297 |
| 449 | RQARVVGG | Probe #449 | (FAM)-GRQARVVGGG-K(CPQ2)-(PEG2)- | 1102 |
| | | | DLys-DLys-GC | and |
| | | | | 1297 |
| 450 | RQRRVVGG | Probe #450 | (FAM)-GRQRRVVGGG-K(CPQ2)-(PEG2)- | 1103 |
| | | | DLys-DLys-GC | and |
| | | | | 1297 |
| 451 | RQARAVGG | Probe #451 | (FAM)-GRQARAVGGG-K(CPQ2)-(PEG2)- | 1104 |
| | | | DLys-DLys-GC | and |
| | | | | 1297 |
| 452 | RKRRGSRG | Probe #452 | (FAM)-GRKRRGSRGG-K(CPQ2)-(PEG2)- | 1105 |
| | | | DLys-DLys-GC | and |
| | | | | 1297 |
| 453 | KQSRKFVP | Probe #453 | (FAM)-GKQSRKFVPG-K(CPQ2)-(PEG2)- | 1106 |
| | | | DLys-DLys-GC | and |
| | | | | 1297 |
| 454 | VTGRS | Probe #454 | (FAM)-GVTGRSG-K(CPQ2)-(PEG2)-DLys- | 1107 |
| | | | DLys-GC | and |
| | | | | 1297 |
| 455 | LKSRVK | Probe #455 | (FAM)-GLKSRVKG-K(CPQ2)-(PEG2)-DLys- | 1108 |
| | | | DLys-GC | and |
| | | | | 1297 |
| 456 | GIGAVLKVLT | Probe #456 | (FAM)-GGIGAVLKVLTG-K(CPQ2)-(PEG2)- | 1109 |
| | | | DLys-DLys-GC | and |
| | | | | 1297 |
| 457 | GLPALISWIK | Probe #457 | (FAM)-GGLPALISWIKG-K(CPQ2)-(PEG2)- | 1110 |
| | | | DLys-DLys-GC | and |
| | | | | 1297 |
| 458 | SEVNLDAEF | Probe #458 | (FAM)-GSEVNLDAEFG-K(CPQ2)-(PEG2)- | 1111 |
| | | | DLys-DLys-GC | and |
| | | | | 1297 |
| 459 | EEKPICFFRLG | Probe #459 | (FAM)-GEEKPICFFRLGKEG-K(CPQ2)- | 1112 |
| | KE | | (PEG2)-DLys-DLys-GC | and |
| | | | | 1297 |
| 460 | EEKPILFFRLG | Probe #460 | (FAM)-GEEKPILFFRLGKEG-K(CPQ2)- | 1113 |
| | KE | | (PEG2)-DLys-DLys-GC | and |
| | | | | 1297 |
| 461 | APSSVIAA | Probe #461 | (FAM)-GAPSSVIAAG-K(CPQ2)-(PEG2)- | 1114 |
| | | | DLys-DLys-GC | and |
| | | | | 1297 |
| 462 | KKAKRNAL | Probe #462 | (FAM)-GKKAKRNALG-K(CPQ2)-(PEG2)- | 1115 |
| | | | DLys-DLys-GC | and |
| | | | | 1297 |
| 463 | WTNTSANYNL | Probe #463 | (FAM)-GWTNTSANYNLG-K(CPQ2)- | 1116 |
| | | | (PEG2)-DLys-DLys-GC | and |
| | | | | 1297 |
| 464 | RVRR | Probe #464 | (FAM)-GRVRRG-K(CPQ2)-(PEG2)-DLys- | 1117 |
| | | | DLys-GC | and |
| | | | | 1297 |
| 465 | ERTKR | Probe #465 | (FAM)-GERTKRG-K(CPQ2)-(PEG2)-DLys- | 1118 |
| | | | DLys-GC | and |
| | | | | 1297 |
| 466 | RYQIKPLKSTD | Probe #466 | (FAM)-GRYQIKPLKSTDEG-K(CPQ2)- | 1119 |
| | E | | (PEG2)-DLys-DLys-GC | and |
| | | | | 1297 |
| 467 | WELRHQA- | Probe #467 | (FAM)-GWELRHQA-(Hfe)-RSKG-K(CPQ2)- | 1120 |
| | (Hfe)-RSK | | (PEG2)-DLys-DLys-GC | and |
| | | | | 1297 |
| 468 | SGAFK-C(Me)- | Probe #468 | (FAM)-GSGAFK-C(Me)-LKDGAGG- | 1121 |
| | LKDGAG | | K(CPQ2)-(PEG2)-DLys-DLys-GC | and |
| | | | | 1297 |
| 469 | YVADGW | Probe #469 | (FAM)-GYVADGWG-K(CPQ2)-(PEG2)- | 1122 |
| | | | DLys-DLys-GC | and |
| | | | | 1297 |
| 470 | WEHDGW | Probe #470 | (FAM)-GWEHDGWG-K(CPQ2)-(PEG2)- | 1123 |
| | | | DLys-DLys-GC | and |
| | | | | 1297 |
| 471 | YVADAPV | Probe #471 | (FAM)-GYVADAPVG-K(CPQ2)-(PEG2)- | 1124 |
| | | | DLys-DLys-GC | and |
| | | | | 1297 |
| 472 | RPPGFSA | Probe #472 | (FAM)-GRPPGFSAG-K(CPQ2)-(PEG2)- | 1125 |
| | | | DLys-DLys-GC | and |
| | | | | 1297 |
| 473 | GSPAFLA | Probe #473 | (FAM)-GGSPAFLAG-K(CPQ2)-(PEG2)- | 1126 |
| | | | DLys-DLys-GC | and |
| | | | | 1297 |
| 474 | AGFSLPA | Probe #474 | (FAM)-GAGFSLPAG-K(CPQ2)-(PEG2)- | 1127 |
| | | | DLys-DLys-GC | and |
| | | | | 1297 |
| 475 | RWHTVGLRW | Probe #475 | (FAM)-GRWHTVGLRWEG-K(CPQ2)- | 1128 |
| | E | | (PEG2)-DLys-DLys-GC | and |
| | | | | 1297 |
| | LEQ | Probe #476 | (FAM)-GLEQG-K(CPQ2)-(PEG2)-DLys- | 1129 |
| | | | DLys-GC | and |
| | | | | 1297 |
| 477 | RWPPMGLPWE | Probe #477 | (FAM)-GRWPPMGLPWEG-K(CPQ2)- | 1130 |
| | | | (PEG2)-DLys-DLys-GC | and |
| | | | | 1297 |
| 478 | RPKPVE | Probe #478 | (FAM)-GRPKPVEG-K(CPQ2)-(PEG2)-DLys- | 1131 |
| | | | DLys-GC | and |
| | | | | 1297 |
| 479 | IETD | Probe #479 | (FAM)-GIETDG-K(CPQ2)-(PEG2)-DLys- | 1132 |
| | | | DLys-GC | and |
| | | | | 1297 |
| 480 | VGPDFGR | Probe #480 | (FAM)-GVGPDFGRG-K(CPQ2)-(PEG2)- | 1133 |
| | | | DLys-DLys-GC | and |
| | | | | 1297 |
| 481 | GIEFDSGGC | Probe #481 | (FAM)-GGIEFDSGGCG-K(CPQ2)-(PEG2)- | 1134 |
| | | | DLys-DLys-GC | and |
| | | | | 1297 |
| 482 | GDFLRRV | Probe #482 | (FAM)-GGDFLRRVG-K(CPQ2)-(PEG2)- | 1135 |
| | | | DLys-DLys-GC | and |
| | | | | 1297 |
| | AAL | Probe #483 | (FAM)-GAALG-K(CPQ2)-(PEG2)-DLys- | 1136 |
| | | | DLys-GC | and |
| | | | | 1297 |
| 484 | YATWSMIAAH | Probe #484 | (FAM)-GYATWSMIAAHG-K(CPQ2)- | 1137 |
| | | | (PEG2)-DLys-DLys-GC | and |
| | | | | 1297 |
| 485 | VIMWRLTVGT | Probe #485 | (FAM)-GVIMWRLTVGTG-K(CPQ2)- | 1138 |
| | | | (PEG2)-DLys-DLys-GC | and |
| | | | | 1297 |
| 486 | RRVLALQQEL | Probe #486 | (FAM)-GRRVLALQQELG-K(CPQ2)-(PEG2)- | 1139 |
| | | | DLys-DLys-GC | and |
| | | | | 1297 |
| 487 | LATWPLSGLW | Probe #487 | (FAM)-GLATWPLSGLWG-K(CPQ2)- | 1140 |
| | | | (PEG2)-DLys-DLys-GC | and |
| | | | | 1297 |
| 488 | NTPNWLVNAV | Probe #488 | (FAM)-GNTPNWLVNAVG-K(CPQ2)- | 1141 |
| | | | (PEG2)-DLys-DLys-GC | and |
| | | | | 1297 |
| 489 | SPLAQAVRSSS | Probe #489 | (FAM)-GSPLAQAVRSSSRKG-K(CPQ2)- | 1142 |
| | RK | | (PEG2)-DLys-DLys-GC | and |
| | | | | 1297 |
| 490 | QMPGRLSMAF | Probe #490 | (FAM)-GQMPGRLSMAFG-K(CPQ2)- | 1143 |
| | | | (PEG2)-DLys-DLys-GC | and |
| | | | | 1297 |
| 491 | PLGLR | Probe #491 | (FAM)-GPLGLRG-K(CPQ2)-(PEG2)-DLys- | 1144 |
| | | | DLys-GC | and |
| | | | | 1297 |
| 492 | QRANSIRVTW | Probe #492 | (FAM)-GQRANSIRVTWG-K(CPQ2)-(PEG2)- | 1145 |
| | | | DLys-DLys-GC | and |
| | | | | 1297 |
| 493 | PLAVR | Probe #493 | (FAM)-GPLAVRG-K(CPQ2)-(PEG2)-DLys- | 1146 |
| | | | DLys-GC | and |
| | | | | 1297 |
| 494 | LLAVPAANTV | Probe #494 | (FAM)-GLLAVPAANTV G-K(CPQ2)- | 1147 |
| | | | | and |
| | | | (PEG2)-DLys-DLys-GC | 1297 |
| 495 | GPQGLRGQ | Probe #495 | (FAM)-GGPQGLRGQG-K(CPQ2)-(PEG2)- | 1148 |
| | | | DLys-DLys-GC | and |
| | | | | 1297 |
| 496 | RTGLYLYNST | Probe #496 | (FAM)-GRTGLYLYNSTG-K(CPQ2)-(PEG2)- | 1149 |
| | | | DLys-DLys-GC | and |
| | | | | 1297 |
| 497 | RKKLTQSKFV | Probe #497 | (FAM)-GRKKLTQSKFVGGAEG-K(CPQ2)- | 1150 |
| | GGAE | | (PEG2)-DLys-DLys-GC | and |
| | | | | 1297 |
| 498 | KHYR | Probe #498 | (FAM)-GKHYRG-K(CPQ2)-(PEG2)-DLys- | 1151 |
| | | | DLys-GC | and |
| | | | | 1297 |
| | QAR | Probe #499 | (FAM)-GQARG-K(CPQ2)-(PEG2)-DLys- | 1152 |
| | | | DLys-GC | and |
| | | | | 1297 |
| 500 | PRPFNYL | Probe #500 | (FAM)-GPRPFNYLG-K(CPQ2)-(PEG2)- | 1153 |
| | | | DLys-GC | |
| 501 | APFEMSA | Probe #501 | (FAM)-GAPFEMSAG-K(CPQ2)-(PEG2)- | 1154 |
| | | | DLys-DLys-GC | and |
| | | | | 1297 |
| 502 | APFEFSA | Probe #502 | (FAM)-GAPFEFSAG-K(CPQ2)-(PEG2)- | 1155 |
| | | | DLys-DLys-GC | and |
| | | | | 1297 |
| 503 | PLGFRV | Probe #503 | (FAM)-GPLGFRVG-K(CPQ2)-(PEG2)-DLys- | 1156 |
| | | | GC | |
| 504 | RPLALWRS | Probe #504 | (FAM)-GRPLALWRSG-K(CPQ2)-(PEG2)-GC | 1157 |
| 505 | RPLALEESQ | Probe #505 | (FAM)-GRPLALEESQG-K(CPQ2)-(PEG2)- | 1158 |
| | | | DLys-GC | |
| 506 | RPLALWRSQ | Probe #506 | (FAM)-GRPLALWRSQG-K(CPQ2)-(PEG2)- | 1159 |
| | | | GC | |
| 507 | RNALAVERTA | Probe #507 | (FAM)-GRNALAVERTASG-K(CPQ2)- | 1160 |
| | S | | (PEG2)-GC | |
| 508 | RPKPQQFW | Probe #508 | (FAM)-GRPKPQQFWG-K(CPQ2)-(PEG2)- | 1161 |
| | | | DLys-GC | |
| 509 | SGSNPYKYTA | Probe #509 | (FAM)-SGSNPYKYTA-K(CPQ2)-(PEG2)- | 1162 |
| | | | DLys-DLys-GC | and |
| | | | | 1297 |
| 510 | SGSNPYGYTA | Probe #510 | (FAM)-SGSNPYGYTA-K(CPQ2)-(PEG2)- | 1163 |
| | | | DLys-DLys-GC | and |
| | | | | 1297 |
| 511 | SGTLSELHTA | Probe #511 | (FAM)-SGTLSELHTA-K(CPQ2)-(PEG2)- | 1164 |
| | | | DLys-DLys-GC | and |
| | | | | 1297 |
| 512 | SGTISHLHTA | Probe #512 | (FAM)-SGTISHLHTA-K(CPQ2)-(PEG2)- | 1165 |
| | | | DLys-DLys-GC | and |
| | | | | 1297 |
| 513 | SG-(Orn)-RSHP- | Probe #513 | (FAM)-SG-(Orn)-RSHP-(Hfe)-TLYTA- | 1166 |
| | (Hfe)-TLYTA | | K(CPQ2)-(PEG2)-DLys-GC | |
| 514 | SG-(Orn)- | Probe #514 | (FAM)-SG-(Orn)-RSHG-(Hfe)-FLYTA- | 1167 |
| | RSHG-(Hfe)- | | K(CPQ2)-(PEG2)-DLys-GC | |
| | FLYTA | | | |
| 515 | SGESLAYYTA | Probe #515 | (FAM)-SGESLAYYTA-K(CPQ2)-(PEG2)- | 1168 |
| | | | DLys-DLys-GC | and |
| | | | | 1297 |
| 516 | SGHMHAALTA | Probe #516 | (FAM)-SGHMHAALTA-K(CPQ2)-(PEG2)- | 1169 |
| | | | DLys-DLys-GC | and |
| | | | | 1297 |
| 517 | ILSR-(DIle)- | Probe #517 | (FAM)-GILSR-(DIle)-VGGG-K(CPQ2)- | 1170 |
| | VGG | | (PEG2)-DLys-GC | |
| 518 | ILS-(DArg)- | Probe #518 | (FAM)-GILS-(DArg)-(DIle)-(DVal)-GGG- | 1171 |
| | (DIle)-(DVal)- | | K(CPQ2)-(PEG2)-DLys-GC | |
| | GG | | | |
| 519 | RQRRALEK | Probe #519 | 5FAM-GRQRRALEKG-K(CPQ2)-PEG2-GC | 1172 |
| 520 | KPISLISS | Probe #520 | 5FAM-GKPISLISSG-K(CPQ2)-PEG2-GC | 1173 |
| 521 | QKGRYKQE | Probe #521 | 5FAM-GQKGRYKQEG-K(CPQ2)-PEG2-GC | 1174 |
| 522 | GPLGLRSW | Probe #522 | 5FAM-GGPLGLRSWK(CPQ2)-PEG2-C | 1175 |
| 523 | GPLGVRGK | Probe #523 | 5FAM-GGPLGVRGKK(CPQ2)-PEG2-C | 1176 |
| 524 | GfPRSGG | Probe #524 | 5FAM-GGfPRSGGGK(CPQ2)-PEG2-C | 1177 |
| | Pyr | Probe #525 | Pyr-AMC | |
| | SY | Probe #526 | H-Ser-Tyr-AMC | |
| | GF | Probe #527 | H-Gly-Phe-AMC | |
| | Y | Probe #528 | H-Tyr-AMC | |
| | Cit | Probe #529 | H-Cit-AMC Hydrobromide salt | |
| | GP | Probe #530 | Suc-Gly-Pro-AMC | |
| | T | Probe #531 | H-Thr-AMC | |
| | I | Probe #532 | H-Ile-AMC | |
| | GA | Probe #533 | H-Gly-Ala-AMC hydrochloride salt | |
| | Cys(Bzl) | Probe #534 | H-Cys(Bzl)-AMC | |
| | A | Probe #535 | H-Ala-AMC | |
| | K | Probe #536 | Ac-Lys-AMC acetate salt | |
| | GLF | Probe #537 | MeOSuc-Gly-Leu-Phe-AMC | |
| | L | Probe #538 | H-Leu-AMC | |
| | VAN | Probe #539 | Z-Val-Ala-Asn-AMC | |
| | AAA | Probe #540 | Suc-Ala-Ala-Ala-AMC | |
| | K | Probe #541 | H-Lys-AMC acetate salt | |
| | F | Probe #542 | H-Phe-AMC trifluoroacetate salt | |
| | FSR | Probe #543 | Boc-Phe-Ser-Arg-AMC | |
| | VVR | Probe #544 | Z-Val-Val-Arg-AMC hydrochloride salt | |
| | KA | Probe #545 | H-Lys-Ala-AMC hydrochloride salt | |
| | PR | Probe #546 | H-Pro-Arg-AMC hydrochloride salt | |
| | MGP | Probe #547 | H-Met-Gly-Pro-AMC hydrochloride salt | |
| | KP | Probe #548 | H-Lys-Pro-AMC hydrochloride salt | |
| | QGR | Probe #549 | Boc-Gln-Gly-Arg-AMC hydrochloride salt | |
| | Glu(OBzl)-AR | Probe #550 | Boc-Glu(OBzl)-Ala-Arg-AMC hydrochloride | |
| | | | salt | |
| 551 | WEHD | Probe #551 | Ac-Trp-Glu-His-Asp-AMC | 1178 |
| | QAR | Probe #552 | Boc-Gln-Ala-Arg-AMC hydrochloride salt | |
| | AAF | Probe #553 | H-Ala-Ala-Phe-AMC (free base) | |
| | GPK | Probe #554 | Tos-Gly-Pro-Lys-AMC | |
| | | | trifluoroacetate salt | |
| 555 | AAPM | Probe #555 | MeOSuc-Ala-Ala-Pro-Met-AMC | 1179 |
| 556 | AEPF | Probe #556 | Suc-Ala-Glu-Pro-Phe-AMC | 1180 |
| | GG | Probe #557 | H-Gly-Gly-AMC hydrochloride salt | |
| | VLK | Probe #558 | Boc-Val-Leu-Lys-AMC acetate salt | |
| | EKK | Probe #559 | Boc-Glu-Lys-Lys-AMC acetate salt | |
| | VPR | Probe #560 | Boc-Val-Pro-Arg-AMC hydrochloride salt | |
| | GKR | Probe #561 | Boc-Gly-Lys-Arg-AMC hydrochloride salt | |
| | Glu(OBzl)-GR | Probe #562 | Boc-Glu(OBzl)-Gly-Arg-AMC hydrochloride | |
| | | | salt | |
| | LR | Probe #563 | Z-Leu-Arg-AMC hydrochloride salt | |
| | AFK | Probe #564 | MeOSuc-Ala-Phe-Lys-AMC trifluoroacetate | |
| | | | salt | |
| | LGR | Probe #565 | Boc-Leu-Gly-Arg-AMC acetate salt | |
| | PFR | Probe #566 | H-Pro-Phe-Arg-AMC acetate salt | |
| 567 | AAPV | Probe #567 | Suc-Ala-Ala-Pro-Val-AMC | 1181 |
| | AFK | Probe #568 | H-Ala-Phe-Lys-AMC trifluoroacetate salt | |
| | VKM | Probe #569 | Z-Val-Lys-Met-AMC acetate salt | |
| 570 | GPLGP | Probe #570 | Suc-Gly-Pro-Leu-Gly-Pro-AMC | 1182 |
| 571 | KQKER | Probe #571 | Ac-Lys-Gln-Lys-Leu-Arg-AMC | 1183 |
| | | | trifluoroacetate salt | |
| 572 | RVRR | Probe #572 | Boc-Arg-Val-Arg-Arg-AMC acetate salt | 1184 |
| 573 | IEGR | Probe #573 | Boc-Ile-Glu-Gly-Arg-AMC acetate salt | 1185 |
| | GP | Probe #574 | H-Gly-Pro-AMC HBr | |
| 575 | AAPV | Probe #575 | MeOSuc-Ala-Ala-Pro-Val-AMC | 1186 |
| 576 | RPFHLL VY | Probe #576 | Suc-Arg-Pro-Phe-His-Leu-Leu-Val-Tyr-AMC | 1187 |
| | | | trifluoroacetate salt | |
| 577 | Anb-WS-Gnf- | Probe #577 | H-Anb-Trp-Ser-Gnf-Thr-Val-Phe-AMC | 1188 |
| | TVF | | | |
| 578 | HSSKLQ | Probe #578 | Mu-His-Ser-Ser-Lys-Leu-Gln-AMC | 1189 |
| | RPY | Probe #579 | MeO-Succ-Arg-Pro-Tyr-AMC | |
| 580 | DRENSPK(Dnp) | Probe #580 | (ACC)-kkDRENSPK(Dnp)L | 1190 |
| | L-OH | | | |
| 581 | kkDRENSPLK( | Probe #581 | (ACC)-kkDRENSPLK(Dnp) | 1191 |
| | Dnp)-OH | | | |
| 582 | NAGSKFK(Dnp | Probe #582 | (ACC)-NAGSKFK(Dnp)Q | 1192 |
| | )Q-OH | | | |
| 583 | NAGSKFQK(Dn | Probe #583 | (ACC)-NAGSKFQK(Dnp) | 1193 |
| | p)-OH | | | |
| 584 | HLLGFYK(Dnp) | Probe #584 | (ACC)-kkHLLGFYK(Dnp)V | 1194 |
| | V-OH | | | |
| 585 | HLLGFYVK(Dn | Probe #585 | (ACC)-kkHLLGFYVK(Dnp) | 1195 |
| | p)-OH | | | |
| 586 | QEKQT(Nle)K( | Probe #586 | (ACC)-kkQEKQT(Nle)K(Dnp)(Nle) | 1196 |
| | Dnp)(Nle)-OH | | | |
| 587 | QEKQT(Nle)(NI | Probe #587 | (ACC)-kkQEKQT(Nle)(Nle)K(Dnp) | 1197 |
| | e)K(Dnp)-OH | | | |
| 588 | DPFVVSK(Dnp) | Probe #588 | (ACC)-kDPFVVSK(Dnp)W | 1198 |
| | W-OH | | | |
| 589 | DPFVVSWK(Dn | Probe #589 | (ACC)-kDPFVVSWK(Dnp) | 1199 |
| | p)-OH | | | |
| 590 | NAYNEIK(Dnp) | Probe #590 | (ACC)-NAYNEIK(Dnp)R | 1200 |
| | R-OH | | | |
| 591 | NAYNEIRK(Dn | Probe #591 | (ACC)-NAYNEIRK(Dnp) | 1201 |
| | p)-OH | | | |
| 592 | V(Nle)RQSEK( | Probe #592 | (ACC)-V(Nle)RQSEK(Dnp)N | 1202 |
| | Dnp)N-OH | | | |
| 593 | V(Nle)RQSENK | Probe #593 | (ACC)-V(Nle)RQSENK(Dnp) | 1203 |
| | (Dnp)-OH | | | |
| 594 | YNPRE(Nle)K( | Probe #594 | (ACC)-YNPRE(Nle)K(Dnp)I | 1204 |
| | Dnp)I-OH | | | |
| 595 | YNPRE(Nle)IK( | Probe #595 | (ACC)-YNPRE(Nle)IK(Dnp) | 1205 |
| | Dnp)-OH | | | |
| 596 | EFVHNPK(Dnp) | Probe #596 | (ACC)-kEFVHNPK(Dnp)K | 1206 |
| | K-OH | | | |
| 597 | EFVHNPKK(Dn | Probe #597 | (ACC)-kEFVHNPKK(Dnp) | 1207 |
| | p)-OH | | | |
| 598 | KRVQFLK(Dnp | Probe #598 | (ACC)-KRVQFLK(Dnp)H | 1208 |
| | )H-OH | | | |
| 599 | KRVQFLHK(Dn | Probe #599 | (ACC)-KRVQFLHK(Dnp) | 1209 |
| | p)-OH | | | |
| 500 | LI(Nle)HKNK(D | Probe #600 | (ACC)-kLI(Nle)HKNK(Dnp)G | 1210 |
| | np)G-OH | | | |
| 601 | LI(Nle)HKNGK( | Probe #601 | (ACC)-KLI(Nle)HKNGK(Dnp) | 1211 |
| | Dnp)-OH | | | |
| 602 | WA(Nle)LYHK( | Probe #602 | (ACC)-kkWA(Nle)LYHK(Dnp)S | 1212 |
| | Dnp)S-OH | | | |
| 603 | WA(Nle)LYHS | Probe #603 | (ACC)-kkWA(Nle)LYHSK(Dnp) | 1213 |
| | K(Dnp)-OH | | | |
| 604 | AHDIVNK(Dnp) | Probe #604 | (ACC)-kkAHDIVNK(Dnp)Y | 1214 |
| | Y-OH | | | |
| 605 | AHDIVNYK(Dn | Probe #605 | (ACC)-kkAHDIVNYK(Dnp) | 1215 |
| | p)-OH | | | |
| 606 | SVFVIEK(Dnp) | Probe #606 | (ACC)-kSVFVIEK(Dnp)P | 1216 |
| | P-OH | | | |
| 607 | SVFVIEPK(Dnp) | Probe #607 | (ACC)-kSVFVIEPK(Dnp) | 1217 |
| | -OH | | | |
| 608 | PPSGLSK(Dnp) | Probe #608 | (ACC)-kPPSGLSK(Dnp)E | 1218 |
| | E-OH | | | |
| 609 | PPSGLSEK(Dnp) | Probe #609 | (ACC)-kPPSGLSEK(Dnp) | 1219 |
| | -OH | | | |
| 610 | RWYGGIK(Dnp) | Probe #610 | (ACC)-kkRWYGGIK(Dnp)F | 1220 |
| | F-OH | | | |
| 611 | RWYGGIFK(Dnp) | Probe #611 | (ACC)-kkRWYGGIFK(Dnp) | 1221 |
| | -OH | | | |
| 612 | QYVFF(Nle)K | Probe #612 | (ACC)-kQYVFF(Nle)K(Dnp)D | 1222 |
| | (Dnp)D-OH | | | |
| 613 | QYVFF(Nle)DK | Probe #613 | (ACC)-kQYVFF(Nle)DK(Dnp) | 1223 |
| | (Dnp)-OH | | | |
| 614 | FAKYYKK(Dnp) | Probe #614 | (ACC)-kFAKYYKK(Dnp)T | 1224 |
| | T-OH | | | |
| 615 | FAKYYKTK(Dnp) | Probe #615 | (ACC)-kFAKYYKTK(Dnp) | 1225 |
| | -OH | | | |
| 616 | QVKHFTK(Dnp) | Probe #616 | (ACC)-kQVKHFTK(Dnp)A | 1226 |
| | A-OH | | | |
| 617 | QVKHFTAK(Dnp) | Probe #617 | (ACC)-kQVKHFTAK(Dnp) | 1227 |
| | -OH | | | |
| 618 | YVADAPK(Dnp) | Probe #618 | (ACC)-KYVADAPK(Dnp) | 1228 |
| | -OH | | | |
| 619 | KGISSQY | Probe #619 | ACC-GKGISSQYK(Dnp)-NH2 | 1229 |
| 620 | ALPALQN | Probe #620 | ACC-GALPALQNK(Dnp)-PEG2-Dlys-Dlys- | 1230 |
| | | | NH2 | |
| 621 | HRFRG | Probe #621 | ACC-GHRFRGK(Dnp)-NH2 | 1231 |
| 622 | APEEIMDQQ | Probe #622 | ACC-GAPEEIMDQQK(Dnp)-PEG2-Dlys- | 1232 |
| | | | Dlys-NH2 | |
| 623 | SRKSQQY | Probe #623 | ACC-GSRKSQQYK(Dnp)-NH2 | 1233 |
| 624 | SKGRSLI | Probe #624 | ACC-GSKGRSLIGK(Dnp)-NH2 | 1234 |
| 625 | FAQSIPK | Probe #625 | ACC-GFAQSIPKK(Dnp)-PEG2-Dlys-Dlys- | 1235 |
| | | | NH2 | |
| 626 | RQRRVVG | Probe #626 | ACC-GRQRRVVGGK(Dnp)-NH2 | 1236 |
| 627 | ERGETGPS | Probe #627 | ACC-GERGETGPSGK(Dnp)-NH2 | 1237 |
| 628 | ASGPSS | Probe #628 | ACC-GASGPSSGK(Dnp)-PEG2-Dlys-Dlys- | 1238 |
| | | | NH2 | |
| 629 | YRFR | Probe #629 | ACC-GYRFRGK(Dnp)-NH2 | 1239 |
| 630 | KLFSSKQ | Probe #630 | ACC-GKLFSSKQK(Dnp)-NH2 | 1240 |
| 631 | IVPRG | Probe #631 | ACC-GIVPRGK(Dnp)-NH2 | 1241 |
| 632 | IRRSSYFK | Probe #632 | ACC-GIRRSSYFKK(Dnp)-NH2 | 1242 |
| 633 | His(Bzl)-Tle- | Probe #633 | ACC-Gly-His(Bzl)-Tle-Pro-Ser-Asp-Met(O)- | 1243 |
| | PSD-Met(O) | | Gly-K(Dnp)-Gly-PEG2-Dlys-Dlys-NH2 | |
| 634 | Nva-IE-Oic- | Probe #634 | ACC-Nva-Ile-Glu-Oic-Asp-Phe-Gly-Arg- | 1244 |
| | DFGR | | Lys(Dnp)-NH2 | |
| | H-DThr- | Probe #635 | Ac-His-DThr-Phe(F5)-Arg-ACC | 1245 |
| | Phe(F5)-R | | | |
| | Dap-Orn- | Probe #636 | Ac-Dap-Orn-Phe(3Cl)-Cys(MeOBzl)-ACC | |
| | Phe(3Cl)- | | | |
| | Cys(MeOBzl) | | | |
| | Cha-L- | Probe #637 | Ac-Cha-Leu-hSer(Bzl)-Arg-ACC | |
| | hSer(Bzl)-R | | | |
| 638 | His(Bzl)-Tle- | Probe #638 | ACC-Gly-His(Bzl)-Tle-Pro-Ser-Asp-Met(O)- | 1246 |
| | PSD-Met(O) | | Gly-K(Dnp)-Gly-PEG2-Dlys-Dlys-NH2 | |
| | hCha-Phe(guan)- | Probe #639 | Ac-hCha-Phe(guan)-Oic-Arg-ACC | |
| | Oic-R | | | |
| | Abu-Nle(O-Bzl) | Probe #640 | NH2-Abu-Nle(O-Bzl)-ACC | |
| | Nle(O-Bzl)- | Probe #641 | Ac-Nle(O-Bzl)-Met(O)2-Oic-Abu-ACC | |
| | Met(O)2-Oic- | | | |
| | Abu | | | |
| | Dap-Orn- | Probe #642 | ACC-G-Dap-Orn-Phe(3Cl)-Cys(MeOBz)-G- | 1247 |
| | Phe(3Cl)- | | K(Dnp)-NH2 | |
| | Cys(MeOBz) | | | |
| | Cha-L-hSer-R | Probe #643 | ACC-Gly-Cha-Leu-hSer-Arg-Gly-K(Dnp)- | 1248 |
| | | | NH2 | |
| 644 | FVT-Gnf-SW | Probe #644 | ACC-Phe-Val-Thr-Gnf-Ser-Trp-K(Dnp)-NH2 | 1249 |
| | hCha-Phe(guan)- | Probe #645 | ACC-Gly-hCha-Phe(guan)-Oic-Arg-Gly- | 1250 |
| | Oic-R | | K(Dnp)-NH2 | |
| | Nle(OBz)- | Probe #646 | ACC-Gly-Nle(OBz)-Met(02)-Oic-Abu-Gly- | |
| | Met(02)-Oic- | | K(Dnp)-NH2 | |
| | Abu | | | |
| 647 | AIEPDSG | Probe #647 | 5FAM-GAIEPDSGG-Lys(CPQ2)-PEG2-Dlys- | 1251 |
| | | | Dlys-GC-NH2 | and |
| | | | | 1298 |
| 648 | AIEFDSG | Probe #648 | 5FAM-GAIEFDSGG-Lys(CPQ2)-Dlys-Dlys- | 1252 |
| | | | GC-NH2 | |
| 649 | AAEAISD | Probe #649 | 5FAM-GGAAEAISDAK(CPQ2)-kk-PEG2-C | 1253 |
| 650 | AGGAQMGA | Probe #650 | 5FAM-GGAGGAQMGAK(CPQ2)-kk-PEG2- | 1254 |
| | | | C | |
| 651 | AQPDALNV | Probe #651 | 5FAM-GGAQPDALNVK(CPQ2)-kk-PEG2-C | 1255 |
| 652 | ATDVTTTP | Probe #652 | 5FAM-GGATDVTTTPK(CPQ2)-kk-PEG2-C | 1256 |
| 653 | DIVTVANA | Probe #653 | 5FAM-GGDIVTVANAK(CPQ2)-kk-PEG2-C | 1257 |
| 654 | DLGLKSVP | Probe #654 | 5FAM-GGDLGLKSVPK(CPQ2)-kk-PEG2-C | 1258 |
| 655 | DVMASNKR | Probe #655 | 5FAM-GGDVMASNKRK(CPQ2)-kk-PEG2-C | 1259 |
| 656 | ESDELNTI | Probe #656 | 5FAM-GGESDELNTIK(CPQ2)-kk-PEG2-C | 1260 |
| 657 | FHPLHSKI | Probe #657 | 5FAM-GGFHPLHSKIK(CPQ2)-kk-PEG2-C | 1261 |
| 658 | HARL VHV | Probe #658 | 5FAM-GGGHARLVHVK(CPQ2)-kk-PEG2-C | 1262 |
| 659 | HIANVERV | Probe #659 | 5FAM-GGHIANVERVK(CPQ2)-kk-PEG2-C | 1263 |
| 660 | KAAATQKK | Probe #660 | 5FAM-GGKAAATQKKK(CPQ2)-kk-PEG2-C | 1264 |
| 661 | LATASTMD | Probe #661 | 5FAM-GGLATASTMDK(CPQ2)-kk-PEG2-C | 1265 |
| 662 | LGPKGQT | Probe #662 | 5FAM-GGLGPKGQTGK(CPQ2)-kk-PEG2-C | 1266 |
| 663 | LSLPETGE | Probe #663 | 5FAM-GGLSLPETGEK(CPQ2)-kk-PEG2-C | 1267 |
| 664 | NLAGILKE | Probe #664 | 5FAM-GGNLAGILKEK(CPQ2)-kk-PEG2-C | 1268 |
| 665 | NPGMSEPV | Probe #665 | 5FAM-GGNPGMSEPVK(CPQ2)-kk-PEG2-C | 1269 |
| 666 | PFGCHAK | Probe #666 | 5FAM-GGPFGCHAKK(CPQ2)-kk-PEG2-C | 1270 |
| 667 | PLGLRWW | Probe #667 | 5FAM-GGPLGLRWWK(CPQ2)-kk-PEG2-C | 1271 |
| 668 | QMGVMQGV | Probe #668 | 5FAM-GGQMGVMQGVK(CPQ2)-kk-PEG2- | 1272 |
| | | | C | |
| 669 | QTCKCSCK | Probe #669 | 5FAM-GGQTCKCSCKK(CPQ2)-kk-PEG2-C | 1273 |
| 670 | QWAGLVEK | Probe #670 | 5FAM-GGQWAGLVEKK(CPQ2)-kk-PEG2-C | 1274 |
| 671 | RPAVMTSP | Probe #671 | 5FAM-GGRPAVMTSPK(CPQ2)-kk-PEG2-C | 1275 |
| 672 | TLRELHLD | Probe #672 | 5FAM-GGTLRELHLDK(CPQ2)-kk-PEG2-C | 1276 |
| 673 | TPPPSQGK | Probe #673 | 5FAM-GGTPPPSQGKK(CPQ2)-kk-PEG2-C | 1277 |
| 674 | TSEDLVVQ | Probe #674 | 5FAM-GGTSEDL VVQK(CPQ2)-kk-PEG2-C | 1278 |
| 675 | VWAAEAIS | Probe #675 | 5FAM-GGVWAAEAISK(CPQ2)-kk-PEG2-C | 1279 |
| | R | Probe #676 | H-R-AMC | |
| | GC | Probe #677 | FAM-GGC-PEG8 | |

| | |
|---|---|
| Nle = norleucine | Oic = L-octahydroindole-2-carboxylic acid |
| K(FAM) = carboxy-fluorescein- | Nva = norvaline |
| L-lysine | (click to see farther down list) |
| HomoPhe = Hfe = L-homophenylalanine | DThr = d-threonine |
| Cys(OMeBzl) = C(OMeBzl) = S-para- | Phe(F5) = 2,3,4,5,6-pentafluoro- |
| methoxybenzyl cysteine | L-penylalanine |
| DIle = d-isoleucine | Phe(3Cl) = 3-chloro-L-phenylalanine |
| DArg = D-arginine | hSer(Bzl) = benzyl homoserine |
| DVal = D-valine | hCha = homocyclohexylalnine |
| Pyr = pyroglutamic acid | Phe(guan) = phenylalanine |
| Cit = citrulline | derivative with a |
| C(Bzl) = S-benzyl-L-cysteine | guanidine group in the para position |
| Glu(OBzl) = benzyl-L-glutamate | Nle(O-Bzl) = Nle(OBz) = benzyloxy-L- |
| Anb = amino-n-butyric acid | norleucine |
| Gnf = guamidine-L-phenylalanine | Met(O)2 = L-methionine sulfone |
| K(Dnp) = dinitrobenzylation of | Dap = 2,3-diaminopropionic acid |
| lysine | hSer = homoserine |
| His(Bzl) = benzyl-L-histidine | Met(02) = methylsulfonylbutanoic acid |
| Tle = L-tert-leucine | Abu = L-alpha-aminobutyric acid |
| Met(O) = L-methionine-sulfoxide | Cha = L-cyclohexylalanine |
| Bz = Benzoyl | Cys(Me) = L- Methyl cysteine |
| | Orn = L-Ornithine |
| | hF = L-Homophenylalanine |
| | GABA = gamma aminobutyric acid |
| | Pip = piperidine carboxylic acid |
| | lower case = D-amino acids |

In some embodiments, the peptide linkers described herein for endoproteases comprise a design: XₘAYₙ or AXₙB, wherein respectively, A comprises a single amino acid and A and B are amino acid pairs recognized by a particular endoprotease, X and Y comprise any amino acid labeled or not with a reporter, and m and n comprise any integer, including zero. This design is for exemplification only and should not be construed as the only possible design for the peptide linker.

In some embodiments, the peptide linkers described herein for exoproteases comprise a design: XₘAYₙ, wherein A comprises amino acid pairs recognized by a particular exoprotease, X and Y comprise any amino acid labeled or not with a reporter, and n and m comprise any integer, including zero. This design is for exemplification only and should not be construed as the only possible design for the peptide linker.

TABLE 2
Example peptide linker designs.

| | | | | | | | | | Critical |
|---|---|---|---|---|---|---|---|---|---|
| amino | amino | amino | amino | amino | | | | | amino |
| acid | acid | acid | acid | acid | Example | | SEQ | | acid |
| in | in | in | in | in | probe | | ID | Protease | (single |
| P1' | P1 | P2 | P3 | P4 | name | Example prob design | NO | family | or pair) |
| | R/K | | | | Probe | (FAM)-GWYKTQYGK(CPQ2)- | 1353 | Endo | Single |
| | | | | | #161 | NH2 | | | |
| | R/K | | | | Probe | (FAM)-GFARRWGGK(CPQ2)- | 1354 | Endo | Single |
| | | | | | #109 | PEG2-k-NH2 | | | |
| | F/Y/L/W | | | | Probe | (FAM)- | 1355 | Endo | Single |
| | | | | | #165 | GSYWP(Nle)QGK(CPQ2)- | | | |
| | | | | | | PEG2-k-NH2 | | | |
| | F/Y | | | | Probe | (FAM)-GFIY(Nle)PTGK(CPQ2)- | 1356 | Endo | Single |
| | | | | | #140 | PEG2-k-NH2 | | | |
| | P | | | | Probe | (FAM)-GTGPKGNGK(CPQ2)- | 825 | Endo | Single |
| | | | | | #148 | NH2 | | | |
| F | K | | | | Probe | (FAM)- | 894 | Endo | Pair |
| | | | | | #217 | GWSKFW(Nle)GK(CPQ2) | | | (AB) |
| D | G | | | | Probe | (FAM)-GKTGDARGK(CPQ2)- | 871 | Endo | Pair |
| | | | | | #194 | PEG2-k-NH2 | | | (AB) |
| L | P | | | | Probe | (FAM)-GGHPLSPGK(CPQ2)- | 952 | Endo | Pair |
| | | | | | #275 | PEG2-kk-NH2 | | | (AB) |
| | D | T/I/V | | | Probe | (FAM)-GVIDKDFGK(CPQ2)- | 1357 | Endo | Pair |
| | | | | | #297 | NH2 | | | (AB) |
| | R | K/R | | | Probe | (FAM)-GFARRWGGK(CPQ2)- | 1358 | Endo | Pair |
| | | | | | #109 | PEG2-k-NH2 | | | (AB) |
| S | R | | | | Probe | (FAM)-GPVRSTNGK(CPQ2)- | 881 | Endo | Pair |
| | | | | | #204 | NH2 | | | (AB) |
| | D | | E | | Probe | (FAM)-GENDRLPGK(CPQ2)- | 876 | Endo | Pair |
| | | | | | #199 | NH2 | | | (near |
| | | | | | | | | | neighbor |
| | | | | | | | | | AXB) |
| | D | | V | | Probe | (FAM)-GQWVDEDGK(CPQ2)- | 925 | Endo | Pair |
| | | | | | #248 | PEG2-k-NH2 | | | (near |
| | | | | | | | | | neighbor |
| | | | | | | | | | AXXB) |
| | K/R at | | | | Probe | (FAM)-kGEFVHNPK(CPQ2)K- | 1359 | Exo | Single |
| | C- | | | | #321 | OH | | | |
| | terminus | | | | | | | | |
| | K/R/H at | | | | Probe | (FAM)-GNAYNEIK(CPQ2)R- | 1360 | Exo | Single |
| | C- | | | | #315 | OH | | | |
| | terminus | | | | | | | | |
| | W/G/F | | | | Probe | NH2- | 1361 | Exo | Single |
| | at N- | | | | #346 | WK(FAM)NAGSKFGKK(CPQ2)- | | | |
| | terminus | | | | | NH2 | | | |
| | Q/K at | | | | Probe | NH2- | 1362 | Exo | Single |
| | N- | | | | #362 | QK(FAM)KRVQFLGK(CPQ2)- | | | |
| | terminus | | | | | NH2 | | | |

In some embodiments, the cleavable linker comprises a carbohydrate. Tung et al. reported a conjugate of β-galactoside and 7-hydroxy-9H-(1,3-dichloro-9,9-dimethylacridin-2-one), which has far-red fluorescence properties after a cleavage by β-galactosidase. Tung C H, Zeng Q, Shah K, Kim D E, Schellingerhout D, Weissleder R. In vivo imaging of beta-galactosidase activity using far red fluorescent switch. Cancer Res. 2004 Mar. 1; 64(5):1579-83. Ho et al. reported combining β-galactosidase substrate with p-benzyloxycarbonyl as a self-immolative linker. β-D-Galactopyranoside, the substrate of β-galactosidase, was conjugated to an optical probe through a para-substituted benzyloxycarbonyl group (serves as a first self-immolative linker) and a glycine residue (serves as a quencher and a second self-immolative linker). Enzymatic cleavage of the β-D-Galactopyranoside triggered a series of spontaneous reactions that resulted in a release of optically active probe. Ho, N.-H., Weissleder, R. and Tung, C.-H. (2007), A Self-Immolative Reporter For β-Galactosidase Sensing. ChemBioChem, 8: 560-566. Some carbohydrate linkers are commercially available.

In some embodiments, the cleavable linker comprises a nucleic acid. The effect of a DNA linker on the behavior of its conjugate both reduces the toxicity of the free drug by reducing its cell penetration, which is positive in case of premature deconjugation in the bloodstream and increases the off-target toxicity on low antigen-expressing cells, presumably due to nonspecific interaction of the nucleic acid-based linker with the cell surface. For example, in an antibody-drug conjugate, the antibody and drug can be non-covalently connected using complementary DNA linkers. Dovgan, I., Ehkirch, A., Lehot, V. et al. On the use of DNA as a linker in antibody-drug conjugates: synthesis, stability and in vitro potency. Sci Rep 10, 7691 (2020). Dovgan et al. disclosed a trastuzumab to be connected to monomethyl auristatin E (MMAE) through a 37-mer oligonucleotide.

In some embodiments, the cleavable linker comprises a lipid. In some embodiments, the cleavable linker comprises a phospholipid. The insertion of phospholipid groups between two fluorescent dyes or a dye/quencher pair allows the detection of phospholipase cleavage activity. In some embodiments, the cleavable linker comprises a phosphodiester. The insertion of phosphodiester groups between two fluorescent dyes or a dye/quencher pair allows the detection of phosphodiesterase cleavage activity. In some embodiments, the lipid is directly attached to the fluorophore: once the covalent bond between the lipid and fluorophore is cleaved, the increase of fluorescent activity allows for the detection of the enzyme presence

In some embodiments, the cleavable linker comprises an ester. Ester groups are often cleaved by saponification. The reactivity of the ester to cleavage can be enhanced by the use of electron-withdrawing groups or stabilized by the use of auto-immolative spacers to precluded spontaneous hydrolysis. In chemical biology, ester-based cleavable compounds were initially used for protein purification and in structural biology. FRET-based probes were designed to image esterase activities.

In some embodiments, the cleavable linker comprises a glycoside. For example, cellulase enzymes deconstruct cellulose to glucose, and are often comprised of glycosylated linkers connecting glycoside hydrolases (GHs) to carbohydrate-binding modules (CBMs).

In some embodiments, the cleavable linker comprises a nucleophile/base sensitive linker. These can include, but are not limited to, halogen nucleophiles, oxygen nucleophiles, safety-catch linkers, thiol nucleophiles, nitrogen nucleophiles, and phenacyl ester derivatives.

In some embodiments, the cleavable linker may be sensitive to activity from all enzyme families, including, but not limited to, oxidoreductases, transferases, hydrolases, lyases, isomerases, and ligases.

Fluoridolyzable linkers are widely used in organic chemistry as silicon-based protecting groups for alcohols. The high thermodynamic affinity of fluorine for silicon allows their removal in orthogonal and mild conditions using a fluorine source. In this reaction a fluoride ion reacts with silicon as nucleophilic species and the cleavage conditions depend on the steric hindrance of the silicon's alkyl group. Fluoride ions can also trigger bond cleavage due to their basic properties.

Oxygen nucleophiles include sulfone and ester linkers, while safety-catch linkers allow greater control over the timing of the bond breakage, because the linker will remain stable until it is activated for cleavage by a chemical modification.

In secondary amine synthesis or solid phase synthesis, nitrobenzenesulfonamides are known to be cleaved with a thiol nucleophile, like b-mercaptoethanol. Cysteines can be modified by electron-deficient alkynes to form a vinyl sulfide linkage.

Displacement reactions involving a specific nitrogen species as a nucleophile can occur in mild cleavable conditions. These reactions can be classified into two groups; cleavage by aminolysis or exchange reaction. For aminolysis cleavage, examples include the cleavage of a malondialdehyde (MDA) indole derivative by either pyrrolidine or hydrazine, and the cleavage of an ester linker by hydroxylamine or hydrazine. Acylhydrazones44 and hydrazones45,156 can be used as cleavable linkers through transimination in a mildly acidic medium. An amine catalyst (e.g., aniline, p-anisidine or hydroxylamine) accelerates hydrolysis and enables the effective transition between stable and dynamic states, which is required for cleavage and exchange.

In some embodiments, the cleavable linker comprises a reduction sensitive linker. Reduction sensitive linkages have been used in chemical biology for a long time and it is a commonly used class of cleavable linker. Examples of cleavable linkers sensitive to reductive conditions include: nitroreductases, disulfide bridges and azo compounds. Karan et al. reported a fluorescent probe to detect nitroreductase. Sanu Karan, Mi Young Cho, Hyunseung Lee, Hwunjae Lee, Hye Sun Park, Mahesh Sundararajan, Jonathan L. Sessler, and Kwan Soo Hong. Near-Infrared Fluorescent Probe Activated by Nitroreductase for In Vitro and In Vivo Hypoxic Tumor Detection. Journal of Medicinal Chemistry 2021 64 (6), 2971-2981. In naturally occurring proteins, disulfide bridges generally play a role in maintaining the protein structure. They are known to be efficiently and rapidly cleaved by mild reducing agents like dithiothreitol (DTT), b-mercaptoethanol or tris(2-carboxyethyl)phosphine (TCEP). In chemical biology, disulfide bridges are used in a wide range of applications including functional and structural proteomics, drug delivery, tumor imaging, DNA and protein-DNA complex purifications. The disulfide-based cleavable linker is commonly used due to its straightforward synthesis and rapid cleavage. Azo linkers are very appealing to chemical biologists since they are able to undergo cleavage following treatment with sodium dithionite, a mild and potentially bio-orthogonal reducing agent. The azo compound is reduced into two aniline moieties via an electrochemical reduction mechanism and this allows the use of reducing agents that are commonly used in many biological protocols, such as TCEP, DTT. In chemical biology, azo compounds have been used to cross-link proteins for over a decade and more recently for protein affinity purification.

In some embodiments, the cleavable linker comprises an electrophile/acid sensitive linker. Acid sensitive linkers can be combined with other type of linkers. For example, a first β-galactosidase cleavage of the β-D-Galactopyranoside triggers the self-immolation of a benzyloxycarbonyl group, resulting in a release of optically active probe. Ho, N.-H., Weissleder, R. and Tung, C.-H. (2007), A Self-Immolative Reporter For β-Galactosidase Sensing. ChemBioChem, 8: 560-566. Two different modes of electrophilic cleavage are used in chemical biology: acidic sensitive linkers that are sensitive to proton sources, and alkyl 2-(diphenylphosphino)benzoate derivatives sensitive to azide compounds. Proton sensitive bonds are among the most frequently used cleavable functions in organic chemistry; illustrated by the development of the BOC group which protects amines, or the Merrifield resin used in solid phase synthesis. In organic chemistry, the cleavage conditions that can be tolerated are very flexible regarding the acids” reagents, solvents, temperatures and pH. In contrast, biocompatible acid cleavable linkers must be responsive to minor changes in pH. Strong acidic conditions can lead to the denaturation of proteins and DNA. Biocompatible acid cleavable linkers are chosen for their instability near physiological pH and are often different from the classical protecting groups, which are cleaved with strong acids. Chemical reactions that can break or form bonds in water can be used as the basis of a cleavable linker, for example the Staudinger ligation. This reaction is proceeded by the nucleophilic attack of an alkyl 2-(diphenylphosphino)benzoate derivative on an azide, to form an aza-ylide intermediate. Then the ester traps the aza-ylide, which leads to the formation of an amide. In this process, the ester acts as a cleavable linker, and the azide as a bioorthogonal chemical agent, which guarantees a chemoselective and bioorthogonal cleavage.

In some embodiments, the cleavable linker comprises a metal or metallic cleavable linker. Organometallic compounds are used to catalyze the modification of proteins containing non-natural amino acids, but their use as cleavage reagent in chemical biology has only been reported a few times. The allyl function is a commonly used protecting group for alcohols in organic synthesis and it is also used as a cleavable linker in DNA sequencing by synthesis Metal cleavable linkers were also used in the design of peptide nucleic acids (PNAs), which were developed for enzyme-independent DNA/RNA hybridization methods.

In some embodiments, the cleavable linker comprises an oxidation sensitive linker. Sodium periodate is undoubtedly the most frequently used biocompatible oxidizing agent due to its ability to cleave vicinal diols to form two aldehydes compounds. One example of this type of cleavable linker consists of a vicinal diol with a tartaric acid spacer and two functional groups at both ends. Selenium based linkers also contain cleavable bonds sensitive to oxidizing agents, such as sodium periodate or N-chlorobenzenesulfonamide immobilized on polystyrene beads (iodo-beads). The trigger agent oxidizes the labile bond to selenium oxide, which is then cleaved directly via intramolecular b-elimination or rearrangement.

### Reporter and Detection Methods

In some aspects, the probe/molecule described herein comprises a reporter. The reporter as described herein comprises or is comprised in any structure that may be capable of being detected by any method, including, but not limited to, fluorescent detection, spectroscopic detection, immunological detection or imaging detection. In some embodiments, the reporter comprises a fluorescent label, a mass tag or a nucleic acid barcode.

In some embodiments, the reporter comprises a fluorescent label. Labels, tags and probes containing small compounds such as florescence can be used to label proteins and nucleic acids. Bio-affinity towards other molecules (biotin, digoxygenin), enzymatic (AP, HRP) or chemiluminescent (esters or acridine) can be used as well. Genetically encoded markers like the fluorescent proteins of the GFP family have become a reporter of choice for gene expression studies and protein localization. In combination with subcellular tags, GFP can be used to label subcellular structures like synapses allowing novel approaches to study developmental processes like synapse formation. Other fluorescent labels include but are not limited to small organic dyes and lipophilic dyes. The fluorescence label may serve itself as the activity substrate without addition of linkers.

Some reporters are “internally quenched”, thus do not require a quencher, wherein the cleavage of a bond linking the internally quenched fluorophore to the substrate linker directly yields a fluorescent molecule. Many described probes for proteases, esterases, peroxidases and others function in that manner.

In some embodiments, the reporter comprises a mass tag. Mass tag reagents are designed to enable identification and quantitation of proteins in different samples using mass spectrometry (MS). Mass tagging reagents within a set typically have the same nominal mass (i.e., are isobaric) and chemical structure composed of an amine-reactive NHS ester group, a spacer arm (mass normalizer), and a mass reporter.

In some embodiments, the reporter comprises a nucleic acid barcode. For example, DNA barcoding is a system for species identification focused on the use of a short, standardized genetic region acting as a “barcode” in a similar way that Universal Product Codes are used by supermarket scanners to distinguish commercial products.

In some embodiments, the reporter can be detected using a ligand binding assay. A ligand binding assay often involves a detection step, such as an ELISA, including fluorescent, colorimetric, bioluminescent and chemiluminescent ELISAs, a paper test strip or lateral flow assay, or a bead-based fluorescent assay. In some embodiments, a paper-based ELISA test may be used to detect the cleaved reporter in the fluid sample. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Fluid from a sample taken from the subject may then be added to the test spots. Then, for example, a streptavidin alkaline phosphate (ALP) conjugate may be added to the test spots, as the detection antibody. Bound ALP may then be exposed to a color reacting agent, such as BCIP/NBT (5-bromo-4-chloro-3″-indolyphosphate p-toluidine salt/nitro-blue tetrazolium chloride), which causes a purple-colored precipitate, indicating presence of the reporter.

In some embodiments, the reporter can be detected using volatile organic compounds. Volatile organic compounds may be detected by analysis platforms such as gas chromatography instrument, a breathalyzer, a mass spectrometer, or use of optical or acoustic sensors. Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

Mass spectrometry and enrichment/chromatography methods may be used to separate and distinguish/detect cleaved from intact reporters used in the present invention based on differences in mass and or presence of a label. For example, enzymatic reactions can result in the fragmentation of a parent molecule resulting in a mass shift of the starting substrate, this can be exploited in different chromatography/enrichment methods such as size exclusion chromatography and affinity enrichments. In mass spectrometry, a sample is ionized, for example by bombarding it with electrons. The sample may be solid, liquid, or gas. By ionizing the sample, some of the molecules in the sample are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis may be performed directly on the biological sample or the detectable cleaved reporters may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis. Detecting the cleaved reporters may provide a qualitative assessment (e.g., whether the detectable cleaved reporters, and thus the predetermined protease is present or absent) or a quantitative assessment (e.g., the amount of the detectable cleaved reporters present) to indicate a comparative activity level of the predetermined proteases in the fluid sample. The quantitative value may be calculated by any means, such as, by determining the percent relative amount of each fraction present in the sample. Methods for making these types of calculations are known in the art.

The cleaved reporters may be detected by any detection method that may be suitable for the particular reporter. In some aspects, the detection method comprises fluorescent detection, spectroscopic detection, mass spectrometry, immunological detection or imaging detection. In some aspects, the detection method comprises fluorescence resonance energy transfer (FRET).

In some embodiments, the detection method comprises spectroscopic detection. Spectroscopic methods of detection are very commonly employed in ion chromatography (IC) and are second only to conductivity detection in their frequency of usage. These methods can be divided broadly into the categories of molecular spectroscopic techniques and atomic spectroscopic techniques. Molecular spectroscopy includes UV-visible spectrophotometry, refractive index measurements, and photoluminescence techniques (fluorescence and phosphorescence). Atomic spectroscopy includes atomic emission spectroscopy (using various excitation sources) and atomic absorption spectroscopy. Many of the spectroscopic detection methods can operate in a direct or indirect mode. The definitions of these terms are the same as those used to describe the electrochemical detection modes. That is, direct spectroscopic detection results when the solute ion has a greater value of the measured detection parameter than does the eluent ion. Indirect detection results when the reverse is true.

In some embodiments, the detection method comprises mass spectrometry. Mass spectrometry (MS) is an analytical technique that is used to measure the mass-to-charge ratio of ions. The results are typically presented as a mass spectrum, a plot of intensity as a function of the mass-to-charge ratio.

In some embodiments, the detection method comprises fluorescence resonance energy transfer (FRET). FRET (Fluorescence Resonance Energy Transfer) is a distance dependent dipole-dipole interaction without the emission of a photon, which results in the transfer of energy from an initially excited donor molecule to an acceptor molecule. It allows the detection of molecular interactions in the nanometer range. FRET peptides are labeled with a donor molecule and an acceptor (quencher) molecule. In most cases, the donor and acceptor pairs are two different dyes. The transferred energy from a fluorescent donor is converted into molecular vibrations if the acceptor is a non-fluorescent dye (quencher). When the FRET is terminated (by separating donor and acceptor), an increase of donor fluorescence can be detected. When both the donor and acceptor dyes are fluorescent, the transferred energy is emitted as light of longer wavelength so that the intensity ratio change of donor and acceptor fluorescence can be measured. In order for efficient FRET quenching to take place, the fluorophore and quencher molecules must be close to each other (approximately 10-100 Å) and the absorption spectrum of the quencher must overlap with the emission spectrum of the fluorophore.

### Precipitating Fluorophore

In some aspects, the cleaved reporter comprises a precipitating fluorophore. In some embodiments, the precipitating fluorophore may be HPQ, Cl-HPQ, HTPQ, HTPQA, HBPQ, or HQPQ.

In some embodiments, the precipitating fluorophore comprises HPQ, also known as 2-(2″-hydroxyphenyl)-4(3H)-quinazolinone. HPQ is a small organic dye known for its classic luminescence mechanism through excited-state intramolecular proton transfer (ESIPT), shows strong light emission in the solid state, but no emission in solution. HPQ is found to be strictly insoluble in water and exhibits intense solid-state fluorescence similar to that of tetraphenyl ethylene. Moreover, its essential properties of insolubility and intense solid-state fluorescence can be countered and reversed, by prohibiting the establishment of an internal hydrogen bond between the imine nitrogen and phenolic hydroxyl group.

In some embodiments, the precipitating fluorophore comprises Cl-HPQ. Cl-HPQ is released when HPQF, a water soluble and non-fluorescent molecule, reacts with furin. Cl-HPQ starts to precipitate near the enzyme activity site, and the precipitates emit bright solid-state fluorescence with more than 60-fold fluorescence enhancement. Li et al. In Situ Imaging of Furin Activity with a Highly Stable Probe by Releasing of Precipitating Fluorochrome. Anal. Chem. 2018, 90, 19, 11680-11687.

In some embodiments, the precipitating fluorophore comprises HTPQ. HTPQ is found to be strictly insoluble in water and shows intense fluorescence in the solid state with maximum excitation and emission wavelengths at 410 nm and 550 nm respectively. This makes it far better suited to the use with a confocal microscope. The large Stokes shift of HTPQ contributes additional and highly desirable advantages: increased sensitivity, minimized background fluorescence and enhanced bioimaging contrast. Liu et al. In Situ Localization of Enzyme activity in Live Cells by a Molecular Probe Releasing a Precipitating Fluorochrome. Angew Chem Int Ed Engl. 2017 Sep. 18; 56(39):11788-11792.

In some embodiments, the precipitating fluorophore comprises HTPQA. HTPQA is another enzyme-responsive fluorogenic probe derived from HTPQ. When converted by ALP, the probe releases free HTPQ which starts to precipitate after a very short delay; the precipitate emits bright solid-state fluorescence with more than 100-fold fluorescence enhancement.

In some embodiments, the precipitating fluorophore comprises HBPQ. HBPQ is completely insoluble in water and shows strong yellow solid emission when excited with a 405 nm laser. Liu et al. Precipitated Fluorophore-Based Molecular Probe for In Situ Imaging of Aminopeptidase N in Living Cells and Tumors. Anal. Chem. 2021, 93, 16, 6463-6471, Publication Date: Apr. 14, 2021.

In some embodiments, the precipitating fluorophore comprises HQPQ. HQPQ is, a novel solid-state fluorophore that is insoluble in water. Li et al. Precipitated Fluorophore-Based Probe for Accurate Detection of Mitochondrial Analytes. Anal. Chem. 2021, 93, 4, 2235-2243. Publication Date: Jan. 5, 2021.

The precipitating and non-precipitating fluorophores can be separated from the enzyme substrate by a self-immolative substrate to stabilize the initial probe and ensure that the enzymatic cleavage is transduced via the immolative spacer into the formation of the precipitating fluorophore or the non-internally quenched soluble fluorophore.

### Fluorescent Quencher

In some aspects, the probe/molecule described herein comprises a fluorescent quencher. The fluorescent quencher as described herein may be in any structure that is capable of decreasing the fluorescence intensity of a given substance. In some embodiments, the fluorescent quencher comprises BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl]azo)benzoyl), Dnp (2,4-dinitrophenyl) or Eclipse®.

In some embodiments, the fluorescent quencher comprises a BHQ quencher including, but not limited to, BHQ0, BHQ1, BHQ2, BHQ3, or BBQ650. BHQ, or black hole quencher, dyes work through a combination of FRET and static quenching to enable avoidance of the residual background signal common to fluorescing quenchers such as TAMRA, or low signal-to-noise ratio. The different types of BHQ dyes are used to quench different colored dyes with BHQ1 used to quench green and yellow dyes such as FAM, TET, or HEX and BHQ2 used for quenching orange and red dyes. BHQ dyes are true dark quenchers with no native emission due to their polyacromatic-azo backbone. Substituting electron-donating and withdrawing groups on the aromatic rings produces a complete series of quenchers with broad absorption curves that span the visible spectrum.

In some embodiments, the fluorescent quencher comprises an ATTO quencher including, but not limited to, ATTO 540Q, ATTO 580Q, or ATTO 612Q. ATTO quenchers have characteristic properties of strong absorption (high extinction coefficient) and high photo-stability. ATTO quenchers are often utilized as fluorescent quenchers on amine-labeled nucleotides for FRET experiments.

In some embodiments, the fluorescent quencher comprises CPQ2. The quencher CPQ2 is often used as a pair with the fluorescent donor 5-carboxylfluorescein.

In some embodiments, the fluorescent quencher comprises a QSY quencher including but not limited to QSY-21, QSY-35, QSY-7, or QSY-9. QSY probes are dark quenchers, substances that absorb excitation energy from a fluorophore and dissipate the energy as heat.

In some embodiments, the fluorescent quencher comprises DABCYL (4-([4′-dimethylamino)phenyl]azo)benzoyl). DABCYL is one of the most popular acceptors for developing FRET-based nucleic acid probes and protease substrates. DABCYL dyes are often paired with EDANS in FRET-based fluorescent probes. DABCYL has a broad and intense visible absorption but no fluorescence.

In some embodiments, the fluorescent quencher comprises Dnp (2,4-dinitrophenyl). Dnp is a stable quencher and its absorption spectrum does not change with pH, which makes this group a convenient marker for substrate quantitation in solutions.

In some embodiments, the fluorescent quencher comprises Eclipse®. Eclipse® is a non-fluorescent chromophore and a dark quencher often used in dual-labelled probes. As dark quenchers, Eclipse® absorbs energy without emitting fluorescence. Eclipse® has an absorption range from 390 nm to 625 nm and is capable of effective performance in a wide range of colored FRET probes.

### Carrier

In some aspects, the probe/molecule described herein comprises a carrier. The fluorescent quencher as described herein comprises in any structure. In some embodiments, the carrier comprises a native, labeled or synthetic protein, a synthetic chemical polymer of precisely known chemical composition or with a distribution around a mean molecular weight (e.g. a linear or branched PEG polymers), an oligonucleotide, a phosphorodiamidate morpholino oligomer (PMO), or a foldamer, a lipid, a lipid micelle, a nanoparticle (e.g., iron oxide, gold, and non-metallic nanoparticles), a solid support made of polystyrene, polypropylene or any other type of plastic or polymer. In some embodiments, the carrier comprises a peptide longer than the peptide linker. A carrier can be covalently or non-covalently attached to the cleavable linker.

In some embodiments, the carrier comprises a nanoparticle. The transport of insoluble drugs via nanoparticles is improving because of their small particle size. Nanoparticle carrier is a kind of sub-micro particle delivery system, which belongs to a nanoscale microscope. Drugs encapsulated in sub-particles can adjust the speed of drug release, increase the permeability of biofilm, change the distribution in vivo, and improve the bioavailability. Nanoparticles are solid colloidal particles ranging in size from 10 to 100 nm used as a core in functionalization systems. They are generally composed of natural or synthetic macromolecule substances and can be used as carriers for conducting or transporting drugs. Nanospheres and nanocapsules can be formed. The chemical materials of nanomaterials are chitosan, gelatin, branched polymers, carbon-based carriers, etc. Gold nanoparticles consist of a core of gold atoms that can be functionalized by addition of a monolayer of moieties containing a thiol (SH) group.

In some embodiments, the carrier comprises a native, labeled or synthetic protein. Proteins can be used as carriers for the delivery of chemicals and biomolecular drugs, such as anticancer drugs and therapeutic proteins. Protein nanoparticles have several advantages as a drug delivery system, such as biodegradability, stability, surface modification of particles, ease of particle size control, and they have less problems associated with toxicity issues, such as immunogenicity. Protein nanoparticles can be generated using proteins, such as fibroins, albumin, gelatin, gliadine, legumin, 30Kc19, lipoprotein, and ferritin proteins, and are prepared through emulsion, electrospray, and desolvation methods. Hong S, Choi D W, Kim H N, Park C G, Lee W, Park H H. Protein-Based Nanoparticles as Drug Delivery Systems. Pharmaceutics. 2020; 12(7):604. Published 2020 Jun. 29. For example, albumin, a plasma protein with a molecular weight of 66 kDa, has been extensively investigated as a drug carrier.

In some embodiments, the carrier comprises a synthetic chemical polymer. Polymeric nanoparticles have been extensively investigated as drug nanocarriers. Drug loading is achieved either by (i) entrapment of an aqueous drug phase using the polymer to form nanoscale structures such as cages and capsules or (ii) chemical linking of the drug molecules to the polymer backbone by means of a simple ester or amide bond that can be hydrolyzed in vivo. The most widely researched synthetic polymers include polylactide (PLA), poly(D,L-lactide-co-glycolide) (PLGA) and PEG. All three polymers are hydrolyzed in vivo and are biodegradable. Malam Y, Loizidou M, Seifalian A M. Liposomes and nanoparticles: nanosized vehicles for drug delivery in cancer. Trends Pharmacol Sci. 2009 November; 30(11):592-9.

In some embodiments, the carrier comprises a polyethylene glycol (PEG). PEG has been studied comprehensively as a carrier because it is soluble in both organic and hydrophilic solvents. Unlike many other synthetic polymers, PEG is relatively hydrophilic. In some embodiments, the PEG comprises a PEG-2, PEG-3, PEG-4, PEG-5, PEG-6, PEG-7, or PEG-8. Conjugation with a PEG increases the solubility of hydrophobic molecules and prolongs the circulation time in the organism. PEG also minimizes the nonspecific absorption of a molecule, such as a drug, provides specific affinity toward the targeted tissue, and increases the drug accumulation in malignant tissue. PEG can be conjugated to other polymers to make them less hydrophobic (i.e., PEGylation). The changes in surface hydrophilicity prevent protein adsorption, thereby enabling cell adhesion and proliferation on biomaterial scaffolds. The PMO backbone is made of morpholino rings with phosphorodiamidate linkage, which protects them from nuclease degradation while still maintaining the complementary base pairing. The potential application of PMO-based antisense technology targeting bacterial pathogens is being explored for the development of a new class of antibacterial drugs. Panchal R G, Geller B L, Mellbye B, Lane D, Iversen P L, Bavari S. Peptide conjugated phosphorodiamidate morpholino oligomers increase survival of mice challenged with Ames Bacillus anthracis. Nucleic Acid Ther. 2012; 22(5):316-322. Fluorescein-tagged Morpholinos combined with fluorescein-specific antibodies can be used as probes for in-situ hybridization to miRNAs.

In some embodiments, the carrier comprises an oligonucleotide. Biostable, high-payload DNA nanoassemblies of various structures, including cage-like DNA nanostructure, DNA particles, DNA polypods, and DNA hydrogel, have been reported. Cage-like DNA structures hold drug molecules firmly inside the structure and leave a large space within the cavity. These DNA nanostructures use their unique structure to carry abundant CpG, and their biocompatibility and size advantages to enter immune cells to achieve immunotherapy for various diseases. Part of the DNA nanostructures can also achieve more effective treatment in conjunction with other functional components such as aPD1, RNA, TLR ligands. DNA-based nanoparticles, such as spherical nucleic acids, hybrid DNA-based nanoparticles, polypod-like DNA nanostructure, DNA hydrogels have been reported. Chi Q, Yang Z, Xu K, Wang C and Liang H (2020) DNA Nanostructure as an Efficient Drug Delivery Platform for Immunotherapy. Front. Pharmacol. 10:1585.

In some embodiments, the carrier comprises a phosphorodiamidate Morpholino oligomer (PMO). Antisense phosphorodiamidate morpholino oligomers (PMOs) and their derivatives downregulate target gene expression in a sequence-dependent manner by interfering with the binding of ribosome to mRNA and thereby inhibiting protein translation.

In some embodiments, the carrier comprises a lipid or a lipid micelle. The liposome bilayer can be composed of either synthetic or natural phospholipids. The predominant physical and chemical properties of a liposome are based on the net properties of the constituent phospholipids, including permeability, charge density and steric hindrance. The lipid bilayer closes in on itself due to interactions between water molecules and the hydrophobic phosphate groups of the phospholipids. This process of liposome formation is spontaneous because the amphiphilic phospholipids self-associate into bilayers. Drug loading into liposomes can be achieved through (i) liposome formation in an aqueous solution saturated with soluble drug; (ii) the use of organic solvents and solvent exchange mechanisms; (iii) the use of lipophilic drugs; and (iv) pH gradient methods. Malam Y, Loizidou M, Seifalian A M. Liposomes and nanoparticles: nanosized vehicles for drug delivery in cancer. Trends Pharmacol Sci. 2009 November; 30(11):592-9.

In some embodiments, the carrier comprises a solid support made of polystyrene, polypropylene or any other type of plastic. For example, drug delivery properties of microporous polystyrene solid foams have been reported by Canal et al. These materials were obtained by polymerization in the continuous phase of highly concentrated emulsions prepared by the phase inversion temperature method. Their porosity, specific surface and surface topography are associated with drug incorporation and release characteristics. Canal, Cristina & Aparicio, Rosa & Vilchez, Alejandro & Esquena, Jordi & Garcia-Celma, Maria. (2012). Drug Delivery Properties of Macroporous Polystyrene Solid Foams. Journal of pharmacy & pharmaceutical sciences: a publication of the Canadian Society for Pharmaceutical Sciences, Societe canadienne des sciences pharmaceutiques. 15. 197-207.

In some embodiments, the carrier comprises a foldamer. A foldamer is a folded oligomer or polymer with a well-defined conformation. The conformation of foldamers is highly predictable from their primary sequences, therefore, it is possible to arrange functional groups at target positions and it comprises possible to design functional foldamers, such as for efficient cellular uptake. For example, Cell-penetrating peptide (CPP) foldamers are peptide-based foldamers equipped with cell membrane permeabilities. Peptide foldamers contain unnatural amino acids, non-proteinogenic amino acids, which make the peptide adopt a stable secondary structure, especially helical structures, even in short sequences. This property is helpful for the design of amphipathic CPPs with a stable helical structure. Furthermore, peptides containing unnatural amino acids generally exhibit resistance to hydrolysis by proteases, which are abundant throughout the body and in the cells. High stability of the peptide foldamers against enzymatic degradation can lead to their prolonged function in vivo. Makoto Oba, Cell-Penetrating Peptide Foldamers: Drug Delivery Tools. ChemBioChem 10.1002/cbic.201900204.

### Self-Immolative Spacer

In some aspects, the probe/molecule described herein comprises a self-immolative spacer. In some embodiments, the self-immolative spacer comprise a disulfide, a p-amino benzyl alcohol, an a-quinone methide spacer, a hetheroaminebifuncional disulfide, a thiol-based pirydazinediones, a p-aminebenzyloxycarbonyl, a dipeptide, a Gly-Pro (SEQ ID NO: 530), a L-Phe-Sar, a trans-cyclooctene tetrazine, a ortho Hydroxy-protected Aryl sulfate, a phosphoramidate-based spacer, a hydroxybenzyl, a trimethyl carbamate, a quinone methide-based spacer, a cyclizing spacer, a Trimethyl lock, a 2-amino methyl piperidine or an ethylene diamine derived cyclizing spacer. Gonzaga et al. Perspective about self-immolative drug delivery systems. Journal of Pharmaceutical Sciences 109 (2020) 3262-3281.

Cleavage of the cleavable linker by a predetermined protease or enzyme makes the self-immolative spacer dissociate from the precipitating fluorescent or non-fluorescent reporter, thereby resulting in a detectable signal. The cleavable linker of the plurality of probes/molecules comprises cleavable by a predetermined endoprotease in the body fluid sample resulting in auto immolation and reporter release or results in a protease substrate that can be cleaved by a predetermined exopeptidase. In some embodiments, the predetermined exopeptidase is added to the body fluid sample. In some embodiments, the predetermined exopeptidase cleaves the protease substrate, thereby causing the self-immolative spacer to dissociate from the precipitating fluorescent reporter, thereby resulting in a detectable signal.

### Body Fluid Samples

Determination of the disease or condition is based on the rate of formation or amount of the released reporter detected in the body fluid sample. In some embodiments, the body fluid sample comprises blood, serum, plasma, bone marrow fluid, lymphatic fluid, bile, amniotic fluid, mucosal fluid, saliva, urine, cerebrospinal fluid, synovial fluid, semen, ductal aspirate, feces, vaginal effluent, cyst fluid, tissue homogenate, tissue-derived fluid, lachrymal fluid and patient-derived cell line supernatant. In some embodiments, the body fluid sample comprises a rinse fluid. In some embodiments, the rinse fluid comprises a mouthwash rinse, a bronchioalveolar rinse, a lavage fluid, a hair wash rinse, a nasal spray effluent, a swab of any bodily surface, orifice or organ structure applied to saline or any media or any derivatives thereof.

In some embodiments, the body fluid sample comprises blood. Blood is a constantly circulating fluid providing the body with nutrition, oxygen, and waste removal. Blood is mostly liquid, with numerous cells and proteins suspended in it. Blood is made of several main factors including plasma, red blood cells, white blood cells, and platelets.

In some embodiments, the body fluid sample comprises a plasma. Plasma is the liquid that remains when clotting is prevented with the addition of an anticoagulant. Serum is the conventional term in the art for the fluid that remains when clotting factors are removed from plasma. Anticoagulants are medicines that help prevent blood clots. Examples of anticoagulants include, but are not limited to, an ethylenediamine tetraacetic acid (EDTA), a citrate, a heparin, an oxalate, any salt, solvate, enantiomer, tautomer and geometric isomer thereof, or any mixtures thereof.

In some embodiments, the body fluid sample comprises bone marrow fluid. Bone marrow is found in the center of most bones and has many blood vessels. There are two types of bone marrow: red and yellow. Red marrow contains blood stem cells that can become red blood cells, white blood cells, or platelets. Yellow marrow is made mostly of fat.

In some embodiments, the body fluid sample comprises lymphatic fluid. Lymphatic fluid, also called lymph, is a collection of the extra fluid that drains from cells and tissues, that is not reabsorbed into the capillaries.

In some embodiments, the body fluid sample comprises bile. Bile is a digestive fluid produced by the liver and stored in the gallbladder. During bile reflux, digestive fluid backs up into the stomach and, in some cases, the esophagus.

In some embodiments, the body fluid sample comprises amniotic fluid. Amniotic fluid is a clear, slightly yellowish liquid that surrounds the unborn baby (fetus) during pregnancy. It is contained in the amniotic sac.

In some embodiments, the body fluid sample comprises mucosal fluid. Mucosal fluid, also called mucus, is a thick protective fluid that is secreted by mucous membranes and used to stop pathogens and dirt from entering the body. Mucus is also used to prevent bodily tissues from being dehydrated.

In some embodiments, the body fluid sample comprises saliva. Saliva is an extracellular fluid produced and secreted by salivary glands in the mouth.

In some embodiments, the body fluid sample comprises urine. Urine is a liquid by-product of metabolism in humans and in many other animals. Urine flows from the kidneys through the ureters to the urinary bladder.

In some embodiments, the body fluid sample comprises cerebrospinal fluid. Cerebrospinal fluid is a clear fluid that surrounds the brain and spinal cord. It cushions the brain and spinal cord from injury and also serves as a nutrient delivery and waste removal system for the brain.

In some embodiments, the body fluid sample comprises synovial fluid. Synovial fluid, also known as joint fluid, is a thick liquid located between your joints. The fluid cushions the ends of bones and reduces friction when joints are moved.

In some embodiments, the body fluid sample comprises semen. Semen is the male reproductive fluid which contains spermatozoa in suspension.

In some embodiments, the body fluid sample comprises ductal aspirate. Ductal aspirate, also known as ductal lavage, ductal fluid, or lavage fluid, is fluid collected from a duct, such as the milk duct of the breast.

In some embodiments, the body fluid sample comprises feces. Feces, also known as excrement or stool is waste matter discharged from the bowels after food has been digested.

In some embodiments, the body fluid sample may be vaginal effluent. Vaginal effluent, also known as vaginal discharge, is a clear or whitish fluid that comes out of the vagina.

In some embodiments, the body fluid sample may be lachrymal fluid. Lachrymal fluid, also known as lacrimal fluid, is secreted by the lacrimal glands to lubricate the eye and fight bacteria.

In some embodiments, the body fluid sample comprises tissue homogenate. A tissue homogenate is obtained through mechanical micro-disruption of fresh tissue and the cell membranes are mechanically permeabilized.

### Proteases and Other Agents

The probe/molecule described herein may be cleaved by a protease from the body fluid. In some embodiments, the protease comprises an endopeptidase or an exopeptidase.

In some embodiments, the protease comprises an endopeptidase. An endopeptidase is an enzyme which breaks peptide bonds other than terminal ones in a peptide chain.

In some embodiments, the protease comprises an exopeptidase. An exopeptidase is an enzyme that catalyzes the cleavage of the terminal or penultimate peptide bond; the process releases a single amino acid or dipeptide from the peptide chain.

In some embodiments, the protease comprises an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enh. binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, or a combination thereof.

In some embodiments, the protease comprises a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), or a combination thereof.

In some embodiments, the protease comprises a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, or a combination thereof.

In some embodiments, the protease comprises a DDI-related protease, a DECYSIN, a Derl-like domain family, member 1, a Derl-like domain family, member 2, a Derl-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

In some embodiments, the protease comprises an enamelysin, an endopeptidase Clp, an endoplasmic reticulum metallopeptidase 1, an endothelin-converting enzyme 1, an endothelin-converting enzyme 2, an enteropeptidase, an epidermis-specific SP-like, an epilysin, an epithelial cell transforming sequence 2 oncogene-like, an epitheliasin, an epoxide hydrolase, an epoxyde hydrolase related protein, an eukar. translation initiation F3SF, an eukar. translation initiation F3SH, or a combination thereof.

In some embodiments, the protease comprises a Factor VII activating protease, a FACE-1/ZMPSTE24, a FACE-2/RCE1, a family with sequence similarity 108, member A1, a family with sequence similarity 108, member B1, a family with sequence similarity 108, member C1, a family with sequence similarity 111, A, a family with sequence similarity 111, B, a furin, or a combination thereof.

In some embodiments, the protease comprises a gamma-glutamyl hydrolase, a gamma-glutamyltransferase 1, a gamma-glutamyltransferase 2, a gamma-glutamyltransferase 5, a gamma-glutamyltransferase 6, a gamma-glutamyltransferase m-3, a gamma-glutamyltransferase-like 3, a GCDFP15, a gelatinase A, a gelatinase B, a Gln-fructose-6-P transamidase 1, a Gln-fructose-6-P transamidase 2, a Gln-fructose-6-P transamidase 3, a Gln-PRPP amidotransferase, a glutamate carboxypeptidase II, a glutaminyl cyclase, a glutaminyl cyclase 2, a glycosylasparaginase, a glycosylasparaginase-2, a granzyme, a granzyme A, a granzyme B, a granzyme H, a granzyme K, a granzyme M, a haptoglobin-1, or a combination thereof.

In some embodiments, the protease comprises a histone deacetylase (HDAC), a haptoglobin-related protein, a HAT-like 2, a HAT-like 3, a HAT-like 4, a HAT-like 5, a HAT-related protease, HSP90AA1?(a heat shock 90 kDa protein 1, alpha), HSP90AB1?(a heat shock 90 kDa protein 1, beta), a heat shock protein 75, a heat shock protein 90 kDa beta (Grp94), member 1/tumor rejection antigen (gp96), a hepatocyte growth factor, a hepsin, a HetF-like, a HGF activator, a hGPI8, a Hin-1/OTU domain containing 4, a homologue ICEY, a HP43.8KD, a HTRA1 serine protease, a HTRA2, a HTRA3, a HTRA4, a hyaluronan-binding ser-protease, a implantation serine protease 2, a indian hedgehog protein, a insulysin, an intestinal serine protease 1, a josephin-1, a josephin-2, or a combination thereof.

In some embodiments, the protease comprises a Kallikrein (KLK), a kallikrein hK1, a kallikrein hK2, a kallikrein hK3, a kallikrein hK4, a kallikrein hK5, a kallikrein hK6, a kallikrein hK7, a kallikrein hK8, a kallikrein hK9, a kallikrein hK10, a kallikrein hK 11, a kallikrein hK12, a kallikrein hK13, a kallikrein hK14, a kallikrein hK15, a Kell blood-group protein, a KHNYN KH and NYN domain containing, a lactotransferrin, a legumain, a leishmanolysin-2, a leucyl aminopeptidase, a leucyl-cystinyl aminopeptidase, a leukotriene A4 hydrolase, a lysosomal carboxypeptidase A, a lysosomal Pro-X C-peptidase, or a combination thereof.

In some embodiments, the protease comprises a membrane metallo-endopeptidase (MME), a macrophage elastase, a macrophage-stimulating protein, a mammalian tolloid-like 1 protein, a mammalian tolloid-like 2 protein, a MAP1D methione aminopeptidase 1D, a marapsin, a marapsin 2, a MASP1/3 (a MBL associated serine protease 3), a MBL associated serine protease 2 (MASP2), a mastin, a matrilysin, a matrilysin-2, a matriptase, a matriptase-2, a matriptase-3, a membrane dipeptidase, a membrane dipeptidase 2, a membrane dipeptidase 3, a membrane-type mosaic Ser-protein, a meprin alpha subunit, a meprin beta subunit, a mesoderm-specific transcript, a mesotrypsin, a methionyl aminopeptidase I, a methionyl aminopeptidase II, a methionyl aminopeptidase II-like, a mitochondrial inner membrane protease 2, a mitochondrial Intermediate peptidase, a mitochondrial Proc. peptidase b-subunit, a mitochondrial proc. protease, a mitochondrial signal peptidase, a matrix metalloproteinase (MMP), a MMP19, a MMP21, a MMP23A, a MMP23B, a MMP27, a MPND, a MT1-MMP, a MT2-MMP, a MT3-MMP, a MT4-MMP, a MT5-MMP, a MT6-MMP, a MYSM1, or a combination thereof.

In some embodiments, the protease comprises a NAALADASE II, a NAALADASE like 2, a NAALADASE like1, a napsin A, a napsin B, a nardilysin, a nasal embryonic LHRH factor, a NEDD4 binding protein 1, a neprilysin, a neprilysin-2, a neurolysin, a neurotrypsin, a neutrophil elastase (ELANE, ELA2), a NLRP1 self-cleaving protein, a nuclear recept. interacting protein 2, a nuclear recept. interacting protein 3, a nucleoporin 98, a NYN domain and retroviral integrase containing, a NY-REN-60, an OMA1, an O-sialoglycoprotein endopeptidase, an O-sialoglycoprotein endopeptidase like 1, an osteoblast serine protease, an OTU domain containing 6B, an OTU domain containing-1, an OTU domain containing-3, an OTU domain containing-5, an OTU domain containing-6A, an otubain-1, an otubain-2, an OTUD2/YOD1, an ovastacin, an oviductin-like/ovochymase-2, an ovochymase-like, or a combination thereof.

In some embodiments, the protease comprises a proteinase 3 (PRTN3), a papain, a PACE4 proprotein convertase, a pancreatic elastase, a pancreatic elastase II (IIA), a pancreatic elastase II form B, a pancreatic endopeptidase E (A), a pancreatic endopeptidase E (B), a pappalysin-1, a pappalysin-2, a paracaspase, a paraplegin, a pepsin A, a pepsin C, a PHEX endopeptidase, a PIDD auto-processing protein unit 1, a PIM1 endopeptidase, a PIM2 endopeptidase, a pitrilysin metalloproteinase 1, a plasma Glu-carboxypeptidase, a plasma kallikrein, a plasma-kallikrein-like 2, a plasma-kallikrein-like 3, a plasma-kallikrein-like 4, a plasmin (plasminogen), a PM20D2 peptidase, a POH1/PSMD14, a polyserase-2, a polyserase-3, a polyserase-I, a Ppnx, a presenilin 1, a presenilin 2, a presenilin homolog 1/SPPL3, a presenilin homolog 2, a presenilin homolog 3/SPP, a presenilin homolog 4/SPPL2B, a presenilin homolog 5, a presenilins-assoc. rhomboid like, a procollagen C-proteinase, a proliferation-association protein 1, a prolyl oligopeptidase, a prolyl oligopeptidase-like, a proprotein convertase 1, a proprotein convertase 2, a proprotein convertase 4, a proprotein convertase 5, a proprotein convertase 7, a proprotein convertase 9 (a proprotein convertase subtilisin/kexin type 9, PCSK9), a prostasin, (a protease, serine, 56), a proteasome alpha 1 subunit, a proteasome alpha 2 subunit, a proteasome alpha 3 subunit, a proteasome alpha 3-like subunit, a proteasome alpha 4 subunit, a proteasome alpha 5 subunit, a proteasome alpha 6 subunit, a proteasome alpha 7 subunit, a proteasome alpha 8 subunit, a proteasome b subunit LMP7-like, a proteasome beta 1 subunit, a proteasome beta 2 subunit, a proteasome beta 3 subunit, a proteasome beta 3-like subunit, a proteasome beta 4 subunit, a proteasome catalytic sub. 1-like, a proteasome catalytic subunit 1, a proteasome catalytic subunit 1i, a proteasome catalytic subunit 2, a proteasome catalytic subunit 2i, a proteasome catalytic subunit 3, a proteasome catalytic subunit 3i, a protein C, a protein C-like, a protein Z, a proteinase 3, a PRPF8, a PSMD7, a pyroglutamyl-peptidase I, a pyroglutamyl-peptidase II, or a combination thereof.

In some embodiments, the protease comprises a reelin, a renin, a retinol binding protein 3, a rhomboid 5 homolog 1, a rhomboid 5 homolog 2, a rhomboid domain containing 1, a rhomboid domain containing 2, a rhomboid, veinlet-like 2, a rhomboid, einlet-like 3, a rhomboid-like protein 1, or a combination thereof.

In some embodiments, the protease comprises a serine protease, a serine protease 3 (PRSS3), a S2P protease, a SAD1, a secernin-1, a secernin-2, a secernin-3, a sentrin (SUMO protease 1), a sentrin (SUMO protease 2), a sentrin (SUMO protease 3), a sentrin (SUMO protease 5), a sentrin (SUMO protease 5-like 1), a sentrin (SUMO protease 6), a sentrin (SUMO protease 7), a sentrin (SUMO protease 8), a sentrin (SUMO protease 9), a sentrin (SUMO protease 11), a sentrin (SUMO protease 12), a sentrin (SUMO protease 13), a sentrin (SUMO protease 14), a sentrin (SUMO protease 15), a sentrin (SUMO protease 16), a sentrin (SUMO protease 17), a sentrin (SUMO protease 18), a sentrin (SUMO protease 19), a separase, a seprase, a serine carboxypeptidase 1, a signalase 18 kDa component, a signalase 21 kDa component, a signalase-like 1, a similar to Arabidopsis Ser-prot., a similar to SPUVE, a site-1 protease, a sonic hedgehog protein, a spinesin, a SprT-like N-terminal domain, a stromelysin 1, a stromelysin 2, a stromelysin 3, a suppressor of Ty 16 homolog, or a combination thereof.

In some embodiments, the protease comprises a taspase, a TBP-associated factor 2, a TESP2, a TESP3, a testase 2, a testis serine protease 2, a testis serine protease 3, a testis serine protease 4, a testis serine protease 5, a testis serine protease 6, a testisin, a testis-specific protein tsp50, a thimet oligopeptidase, a thrombin, a thymus-specific serine peptidase, a TINAG related protein, a TMPRSS11A, a t-plasminogen activator, a TRAF-binding protein domain, a transferrin receptor 2 protein, a transferrin receptor protein, a transmembrane Ser-protease 3, a transmembrane Ser-protease 4, a transthyretin, a TRH-degrading ectoenzyme, a tripeptidyl-peptidase I, a tripeptidyl-peptidase II, a trypsin, a trypsin 10, a trypsin 15, a trypsin C, a trypsin X2, a tryptase, a tryptase alpha/beta 1, a tryptase beta 2, a tryptase delta 1, a tryptase gamma 1, a tryptase homolog 2/EOS, a tryptase homolog 3, a tubulointerstitial nephritis antigen, or a combination thereof.

In some embodiments, the protease comprises a ubiquitin C-term. hydrolase BAP1, a ubiquitin C-terminal hydrolase 1, a ubiquitin C-terminal hydrolase 3, a ubiquitin C-terminal hydrolase 4, a ubiquitin C-terminal hydrolase 5, a ubiquitin specific peptidase like 1, a UCR1, a UCR2, a UDP-N-acetylglucosaminyltransferase subunit, a Ufm-1 specific protease 1, a Ufm-1 specific protease 2, a urokinase (PLAU, uPA) a umbelical vein proteinase, a u-plasminogen activator, a USP1, a USP2, a USβ, a USP4, a USP5, a USP6, a USP7, a USP8, a USP9X, a USP9Y, a USP10, a USP11, a USP12, a USP13, a USP14, a USP15, a USP16, a USP17, a USP17-like, a USP18, a USP19, a USP20, a USP21, a USP22, a USP24, a USP25, a USP26, a USP27, a USP28, a USP29, a USP30, a USP31, a USP34, a USP35, a USP36, a USP37, a USP40, a USP41, a USP42, a USP43, a USP44, a USP45, a USP46, a USP47, a USP48, a USP49, a USP50, a USP51, a USP52, a USP53, a USP54, or a combination thereof.

In some embodiments, the protease comprises a VCP(p97)/p47-interacting protein, a VDU1, a vitellogenic carboxypeptidase-L, a X-Pro dipeptidase, a X-prolyl aminopeptidase 2, a YME1-like 1, a zinc finger CCCH-type containing 12A, a zinc finger CCCH-type containing 12B, a zinc finger CCCH-type containing 12C, a zinc finger CCCH-type containing 12D, a Zinc finger containing ubiquitin peptidase 1, or a combination thereof.

In some embodiments, the protease comprises an A20 (Tumor necrosis factor, alpha-induced protein 3, TNF a-induced protein 3). A20 is a zinc finger protein and a deubiquitinating enzyme. A20 has been shown to inhibit NF-kappa B activation as well as TNF-mediated apoptosis, limit inflammation.

In some embodiments, the protease comprises an Angiotensin-converting enzyme 2 (ACE2). ACE2 is an enzyme attached to the membrane cells located to the membrane of cells located in the intestines, kidney, testis, gallbladder, and heart. ACE2 counters the activity of the related angiotensin-converting enzyme, ACE, by reducing the amount of angiostatin II.

In some embodiments, the protease comprises a cathepsin. The cathepsin may be, but is not limited to, a cathepsin A (CTSA), a cathepsin B (CTSB), a cathepsin C (CTSC), a cathepsin D (CTSD), a cathepsin E (CTSE), a cathepsin H (CTSH), a cathepsin K (CTSK), a cathepsin L (CTSL), a cathepsin S (CTSS), a cathepsin V (CTSV), and a cathepsin Z (CTSZ). Cathepsins are a subset of proteases, many of which become activated in low pH. Cathepsisns comprise serine proteases, cysteine proteases, and aspartyl proteases, among others. Cathepsins have been implicated in cancer, Alzheimer's disease, arthritis, Ebola, pancreatitis, glaucoma, COPD, and other diseases.

In some embodiments, the protease comprises a caspase. The caspase may be, but is not limited to, a caspase 1, a caspase 2, a caspase 3, a caspase 4, a caspase 5, a caspase 6, a caspase 7, a caspase 8, a caspase 9, a caspase 10, a caspase 11, a caspase 12, a caspase 13, and a caspase 14.

In some embodiments, the protease comprises a calpain. The calpain may be, but is not limited to, a calpain 1, a calpain 2, a calpain 3, a calpain 4, a calpain 5, a calpain 6, a calpain 7, a calpain 8, a calpain 9, a calpain 10, a calpain 11, a calpain 12, a calpain 13, a calpain 14, and a calpain 15. Caspases are a family of protease enzymes that play essential roles in programmed cell death and cell homeostasis.

In some embodiments, the protease comprises a cysteine protease. Cysteine proteases, also known as thiol proteases, are hydrolase enzymes that degrade proteins. These proteases share a common catalytic mechanism that involves a nucleophilic cysteine thiol in a catalytic triad or dyad. The cysteine protease family comprises Papain (Carica papaya), bromelain (Ananas comosus), cathepsin K (liverwort), calpain (Homo sapiens), caspase-1 (Rattus norvegicus), separase (Saccharomyces cerevisiae), Adenain (human adenovirus type 2), Pyroglutamyl-peptidase I (Bacillus amyloliquefaciens), Sortase A (Staphylococcus aureus), Hepatitis C virus peptidase 2 (hepatitis C virus), Sindbis virus-type nsP2 peptidase (sindbis virus), Dipeptidyl-peptidase VI (Lysinibacillus sphaericus), DeSI-1 peptidase (Mus musculus), TEV protease (tobacco etch virus), Amidophosphoribosyltransferase precursor (Homo sapiens), Gamma-glutamyl hydrolase (Rattus norvegicus), Hedgehog protein (Drosophila melanogaster) and DmpA aminopeptidase (Ochrobactrum anthropi), etc.

In some embodiments, the protease comprises a complement C1r serine protease (Complement component 1r). In some embodiments, the protease comprises a complement C1s serine protease (Complement component is). C1r along with C1q and C1s form the C1 complex. C1r has very narrow trypsin-like specificity that is responsible for activation of the C1 complex. C1 activation is a two-step process involving (1) C1r intramolecular autoactivation and (2) Cis cleavage by activated C1r. C1r contains a chymotrypsin-like serine protease domain at its C-terminal, and cleaves a single Arg-Ile bond in C1r and in C1s. Zvi Fishelson, in xPharm: The Comprehensive Pharmacology Reference, 2007.

In some embodiments, the protease comprises a chymotrypsin (chymotrypsins A and B, alpha-chymar ophth, avazyme, chymar, chymotest, enzeon, quimar, quimotrase, alpha-chymar, alpha-chymotrypsin A, alpha-chymotrypsin)). Chymotrypsin is a digestive enzyme component of pancreatic juice acting in the duodenum, where it performs proteolysis, the breakdown of proteins and polypeptides. Chymotrypsin preferentially cleaves peptide amide bonds where the side chain of the amino acid N-terminal to the scissile amide bond is a large hydrophobic amino acid (tyrosine, tryptophan, and phenylalanine).

In some embodiments, the protease comprises a chymase (mast cell protease 1, skeletal muscle protease, skin chymotryptic proteinase, mast cell serine proteinase, skeletal muscle protease). Chymases are a family of serine proteases found in mast cells, basophil granulocytes. Chymases show broad peptidolytic activity and are involved in inflammatory response, hypertension and atherosclerosis.

In some embodiments, the protease comprises a dipeptidyl peptidase (DPP). DPP comprises cathepsin C (DPP1), DPP2, DPP3, DPP4, DPP 6, DPP7, DPP8, DPP9, DPP10.

In some embodiments, the protease comprises a DPP4 (adenosine deaminase complexing protein 2, CD26). DPP4 is expressed on cell surface and is associated with immune regulation, signal transduction, and apoptosis. DPP4 is a serine exopeptidase that cleaves X-proline or X-alanine dipeptides from the N-terminus of polypeptides. DPP-4 is known to cleave a broad range of substrates including growth factors, chemokines, neuropeptides, and vasoactive peptides. DPP4 plays a major role in glucose metabolism, is responsible for the degradation of incretins such as GLP-1, and appears to work as a suppressor in the development of some tumors.

In some embodiments, the protease comprises a DPP1 (Cathepsin C, CTSC). DPP1 is a lysosomal exo-cysteine protease belonging to the peptidase C1 family. Cathepsin C appears to be a central coordinator for activation of many serine proteases in immune/inflammatory cells. Cathepsin C catalyzes excision of dipeptides from the N-terminus of protein and peptide substrates.

In some embodiments, the protease comprises a disintegrin and metalloproteinase (ADAM). ADAMs are a family of single-pass transmembrane and secreted metalloendopeptidases. Not all human ADAMs have a functional protease domain. Those ADAMs which are active proteases are classified as sheddases because they cut off or shed extracellular portions of transmembrane proteins.

In some embodiments, the protease comprises an ADAM12 metalloprotease. ADAM12 binds insulin growth factor binding protein-3 (IGFBP-3), appears to be an early Down syndrome marker, and has been implicated in a variety of biological processes involving cell-cell and cell-matrix interactions, including fertilization, muscle development, and neurogenesis.

In some embodiments, the protease comprises a disintegrin and metalloproteinase with thrombospondin motifs (ADAMTS). ADAMTS is a family of multidomain extracellular protease enzymes, comprising ADAMTS1, ADAMTS2, ADAMTS3, ADAMTS4, ADAMTS5 (=ADAMTS11), ADAMTS6, ADAMTS7, ADAMTS8 (or METH-2), ADAMTS9, ADAMTS10, ADAMTS12, ADAMTS13, ADAMTS14, ADAMTS15, ADAMTS16, ADAMTS17, ADAMTS18, ADAMTS19, and ADAMTS20. Known functions of the ADAMTS proteases include processing of procollagens and von Willebrand factor as well as cleavage of aggrecan, versican, brevican and neurocan, making them key remodeling enzymes of the extracellular matrix. They have been demonstrated to have important roles in connective tissue organization, coagulation, inflammation, arthritis, angiogenesis and cell migration.

In some embodiments, the protease comprises an ADAMTS1. ADAMTS1 is a member of the ADAMTS protein family. The expression of ADAMTS1 may be associated with various inflammatory processes, development of cancer cachexia, normal growth, fertility, and organ morphology and function.

In some embodiments, the protease comprises a Factor VII activating protease (FSAP). FSAP is a circulating serine protease with high homology to fibrinolytic enzymes, and may be associated with the regulation of coagulation and fibrinolysis.

In some embodiments, the protease comprises a furin. Furin belongs to the subtilisin-like proprotein convertase family, and is a calcium-dependent serine endoprotease. Furin's substrates includes: proparathyroid hormone, transforming growth factor beta 1 precursor, proalbumin, pro-beta-secretase, membrane type-1 matrix metalloproteinase, beta subunit of pro-nerve growth factor and von Willebrand factor.

In some embodiments, the protease comprises a histone deacetylase (HDAC). HDACs are a class of enzymes that remove acetyl groups (O═C—CH3) from an s-N-acetyl lysine amino acid on a histone, allowing the histones to wrap the DNA more tightly.

In some embodiments, the protease comprises a HTRA1 serine protease. HTRA1 is a secreted enzyme that is proposed to regulate the availability of insulin-like growth factors (IGFs) by cleaving IGF-binding proteins. It has also been suggested to be a regulator of cell growth.

In some embodiments, the protease comprises a granzyme. Granzymes are serine proteases released by cytoplasmic granules within cytotoxic T cells and natural killer (NK) cells. Granzymes induce programmed cell death in the target cell. Granzymes also kill bacteria and inhibit viral replication.

In some embodiments, the protease comprises, a Kallikrein (KLK). Kallikreins are a subgroup of serine proteases. Kallikreins are responsible for the coordination of various physiological functions including blood pressure, semen liquefaction and skin desquamation.

In some embodiments, the protease comprises a matrix metalloproteinase (MMP, matrix metallopeptidases, matrixins). MPPs are calcium-dependent zinc-containing endopeptidases. MMPs have been implicated in cleavage of cell surface receptors, the release of apoptotic ligands, chemokine/cytokine inactivation, cell proliferation and cell migration.

In some embodiments, the protease comprises a membrane metallo-endopeptidase (MME). MME is a zinc-dependent metalloprotease that cleaves peptides at the amino side of hydrophobic residues and inactivates several peptide hormones including glucagon, enkephalins, substance P, neurotensin, oxytocin, and bradykinin. MME is expressed in a wide variety of tissues and is particularly abundant in kidney. MME is also a common acute lymphocytic leukemia antigen.

In some embodiments, the protease comprises a mannose-binding protein-associated serine protease 2 (MASP2, Mannan-binding lectin serine protease 2, MBL associated serine protease 2). MASP2 is involved in the complement system, cleaves complement components C4 and C2 into C4a, C4b, C2a, and C2b.

In some embodiments, the protease comprises a mannose-binding protein-associated serine protease 3 (MBL associated serine protease 3, MASP3). MASP3 originates from the MASP1 gene through differential splicing, it circulates in high serum concentrations predominantly in complex with Ficolin-3 and regulates Ficolin-3 mediated complement activation.

In some embodiments, the protease comprises a neutrophil elastase (ELANE, ELA2). ELANE is a serine proteinase secreted by neutrophils and microphages during inflammation and destroys bacteria and host tissue.

In some embodiments, the protease comprises a proteinase 3 (PRTN3). PRTN3 is a serine protease enzyme expressed mainly in neutrophil granulocytes and contributes to the proteolytic generation of antimicrobial peptides.

In some embodiments, the protease comprises a plasmin (a.k.a. plasminogen). Plasmin is a proteolytic enzyme derived from an inert plasma precursor known as plasminogen. It is present in blood that degrades many blood plasma proteins, including fibrin clots. In human, plasmin is encoded by PLG gene.

In some embodiments, the protease comprises a pepsin. Pepsin is an endopeptidase that cleaves proteins into smaller peptides. It is an aspartic protease, using a catalytic aspartate in its active site.

In some embodiments, the protease comprises a presenilin-1 (PS-1). PS-1 is a presenilin protein that is one of the four core proteins in the gamma secretase complex, which is considered to play an important role in generation of amyloid beta from amyloid precursor protein.

In some embodiments, the protease comprises a proprotein convertase subtilisin/kexin type 9 (PCSK9). PCSK9 is a member of the peptidase S8 family.

In some embodiments, the protease comprises a serine protease. Serine protease cleaves peptide bonds in proteins, in which serine serves as the nucleophilic amino acid at the enzyme's active site. Serine protease includes many subfamilies.

In some embodiments, the protease comprises a tryptase. Tryptase is a the most abundant secretory granule-derived serine proteinase contained in mast cells and has been used as aa marker for mast cell activation. It is released from mask cells when they are activated as part of a normal immune response as well as in allergic responses.

In some embodiments, the protease comprises, a trypsin. Trypsin is a serine protease from the PA clan superfamily, found in the digestive system. Trypsin cuts peptide chains mainly at the carboxyl side of the amino acids lysine or arginine.

In some embodiments, the protease comprises a urokinase (PLAU, uPA). Urokinase is a serine protease present in humans and other animals. It is present in human urine, blood and in the extracellular matrix of many tissues. It is involved in degradation of the extracellular matrix and possibly tumor cell migration and proliferation. Urokinase is a 411-residue protein, consisting of three domains: the serine protease domain, the kringle domain, and the EGF-like domain. Urokinase is synthesized as a zymogen form (prourokinase or single-chain urokinase), and is activated by proteolytic cleavage between Lys158 and Ile159. The two resulting chains are kept together by a disulfide bond.

Described herein are agents to be detected including but are not limited to a oxidoreductase, a transferase, a hydrolase, a lyase, a isomerase, a ligase, a protease, a hydrolase, an esterase, a β-glycosidase, a phospholipase and a phosphodiesterase, peroxidase, lipase, amylase a nucleophilic reagent, a reducing reagent, a electrophilic/acidic reagent, an organometallic/metal catalyst, an oxidizing reagent, a hydroxyl ion, a thiols nucleophile, a nitrogen nucleophile, a sodium dithionite and a sodium periodate.

As described herein, the activity detection of some agents does not rely on cleavage. For example, some oxidoreductases, transferases, hydrolases, lyases, isomerases, and ligases lead to the substrate linker modification and release or formation of a reporter molecule that can be detected. As a way of illustration, a certain oxidation processes can modify an inactive fluorophore and render it fluorescent/detectable without the need of a substrate linker or binding events (for non-covalent processes) can change magnetic/fluorescent physical-chemical properties of certain reporters and render them detectable.

### Disease and Condition

The method described herein comprise determining a disease or condition of the subject. In some aspects, the disease or condition comprises a liver disease, a cancer, a metabolic disease, a fibrotic disease, an organ transplant rejection, an infectious disease, an allergic disease, an autoimmunity, Alzheimer's or a chronic inflammation. In some embodiments, the liver disease comprises a non-alcoholic steatohepatitis (NASH), a non-alcoholic fatty liver disease (NAFLD), a toxin mediated liver injury (drug/medication, alcohol, environmental), a viral hepatitis (HAV, HBV, HCV, HDV, HEV, other virus infecting the liver), an autoimmune hepatitis, a primary biliary cholangitis, a primary sclerosing cholangitis, a fulminant hepatitis, a cirrhosis of the liver, a hepatocellular carcinoma (HCC), a cholangiocarcinoma, an acute or chronic rejection of a transplanted liver, an inherited liver disease (e.g. Wilson disease, hemochromatosis, or alpha-1 antitrypsin) or a combination thereof.

In some embodiments, the cancer comprises adenoid cystic carcinoma, adrenal gland tumors, amyloidosis, anal cancer, appendix cancer, astrocytoma, ataxia-telangiectasia, Beckwith-Wiedemann syndrome, bile duct cancer (cholangiocarcinoma), Birt-Hogg-Dube Syndrome, bladder cancer, bone cancer (sarcoma of the bone), brain stem glioma, brain tumors, breast cancer, Carney complex, central nervous system tumors, cervical cancer, colorectal cancer, Cowden Syndrome, craniopharyngioma, Desmoid tumors, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumors (GIST), germ cell tumors, gestational trophoblastic disease, head and neck cancer, breast and ovarian cancer, diffuse gastric cancer, leiomyosarcoma and renal cell cancer, mixed polyposis syndrome, papillary renal carcinoma, juvenile polyposis syndrome, kidney cancer, lacrimal gland tumors, laryngeal and hypopharyngeal cancer, leukemia, myeloid leukemia, lymphoblastic leukemia, eosinophilic leukemia, Li-Fraumeni syndrome, liver cancer, lung cancer, Hodgkin lung cancer, non-Hodgkin lung cancer, Lynch syndrome, mastocytosis, medulloblastoma, melanoma, meningioma, mesothelioma, multiple endocrine neoplasia, multiple myeloma, myelodysplastic syndrome, nasal cavity and paranasal sinus cancer, nasopharyngeal cancer, neuroblastoma, neuroendocrine tumors, neurofibromatosis, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers syndrome, phenochromocytoma, paraganglioma, pituitary gland tumors, pleuropulmonary blastoma, prostate cancer, retinoblastoma, rhabdomyosarcoma, salivary gland cancer, Kaposi sarcoma, soft tissue sarcoma, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, von Hippel-Lindau syndrome, vulvar cancer, Waldenstrom macroglobulinemia, Werner syndrome, Wilms tumors, or xeroderma pigmentosum.

In some embodiments, the disease comprises a hepatocellular carcinoma. Hepatocellular carcinoma, also referred to as HCC or liver cancer, occurs when a tumor grows in or on the liver. HCC is the most common type of primary liver cancer, and most commonly occurs in people with liver disease, particularly in people with chronic hepatitis B or C. HCC is also more common in people who drink large amounts of alcohol or who have an accumulation of fat in the liver. Symptoms do not often appear in the early stages of the cancer, but symptoms of HCC can include weight loss, upper abdominal pain or yellowing of the skin (jaundice). In some embodiments, the method described herein may distinguish subjects with HCC from healthy subjects. In some embodiments, the method described herein may distinguish subjects with HCC from subjects with cirrhosis of liver only that has not been developed to HCC.

In some embodiments, the disease comprises a certain stage of HCC. Staging systems for HCC have not been universally adopted. One system is the American Joint Committee on Cancer (AJCC) tumor/node/metastasis (TNM) classification system. The TNM classification system takes into account tumor characteristics including size, number, and vascular invasion, as well as lymph node involvement and metastatic disease (Table 3).

TABLE 3
The TNM classification for HCC

| AJCC | Stage | |
|---|---|---|
| Stage | Grouping | Stage Description |
| IA | T1a | A single tumor 2 cm (4/5 in) or smaller that hasn't |
| | | grown into blood vessels |
| | N0 | It has not spread to nearby lymph nodes or to distant |
| | M0 | sites |
| IB | T1b | A single tumor larger than 2 cm (4/5 in) that hasn't |
| | | grown into blood vessels |
| | N0 | It has not spread to nearby lymph nodes or to distant |
| | M0 | sites |
| II | T2 | Either a single tumor larger than 2 cm (4/5 in) that has |
| | | grown into blood vessels, OR more than one tumor but |
| | | none larger than 5 cm (about 2 in) |
| | N0 | It has not spread to nearby lymph nodes or to distant |
| | MC | sites |
| IIIA | T3 | More than one tumor, with at least one tumor larger |
| | | than 5 cm across |
| | N0 | It has not spread to nearby lymph nodes or to distant |
| | M0 | sites |
| IIIB | T4 | At least one tumor (any size) that has grown into a |
| | | major branch of a large vein of the liver (the portal or |
| | | hepatic vein) |
| | N0 | It has not spread to nearby lymph nodes or to distant |
| | M0 | sites |
| IVA | Any T | A single size tumor or multiple tumors of any size |
| | N1 | Tumor has spread to nearby lymph nodes |
| | M0 | Tumor has not spread to distant sites |
| IVB | Any T | A single size tumor or multiple tumors of any size |
| | Any N | May or may not have spread to nearby lymph nodes |
| | M1 | Cancer has metastasized and spread to distant organs |
| | | such as lungs or bone |

In some embodiments, HCC detection using a protease panel can be combined with additional markers of HCC, for example, alpha feto-protein (AFP). AFP, also known as alpha feto-globin, is a major plasma protein produced by the fetus and is thought to be the fetal analog of serum albumin. A sustained increase in AFP serum levels can be a diagnostic marker for HCC and other chronic liver conditions.

### Detection Sensitivity and Specificity

The method described herein comprises a detection sensitivity (also referred to herein as a “sensitivity”). In some cases, the detection sensitivity of the method for HCC may be calculated by the following formula:

Sensitivity=Number of samples with HCC and identified to be positive/(Number of samples with HCC and identified to be positive+Number of samples with HCC and identified to be negative) or Sensitivity=Number of samples with HCC and identified to be positive/Total number of samples with HCC.

In some embodiments, the detection sensitivity is at least about 0.5. In some embodiments, the detection sensitivity is at least about 0.51. In some embodiments, the detection sensitivity is at least about 0.52. In some embodiments, the detection sensitivity is at least about 0.53. In some embodiments, the detection sensitivity is at least about 0.54. In some embodiments, the detection sensitivity is at least about 0.55. In some embodiments, the detection sensitivity is at least about 0.56. In some embodiments, the detection sensitivity is at least about 0.57. In some embodiments, the detection sensitivity is at least about 0.58. In some embodiments, the detection sensitivity is at least about 0.59. In some embodiments, the detection sensitivity is at least about 0.6. In some embodiments, the detection sensitivity is at least about 0.61. In some embodiments, the detection sensitivity is at least about 0.62. In some embodiments, the detection sensitivity is at least about 0.63. In some embodiments, the detection sensitivity is at least about 0.64. In some embodiments, the detection sensitivity is at least about 0.65. In some embodiments, the detection sensitivity is at least about 0.66. In some embodiments, the detection sensitivity is at least about 0.67. In some embodiments, the detection sensitivity is at least about 0.68. In some embodiments, the detection sensitivity is at least about 0.69. In some embodiments, the detection sensitivity is at least about 0.7. In some embodiments, the detection sensitivity is at least about 0.71. In some embodiments, the detection sensitivity is at least about 0.72. In some embodiments, the detection sensitivity is at least about 0.73. In some embodiments, the detection sensitivity is at least about 0.74. In some embodiments, the detection sensitivity is at least about 0.75. In some embodiments, the detection sensitivity is at least about 0.76. In some embodiments, the detection sensitivity is at least about 0.77. In some embodiments, the detection sensitivity is at least about 0.78. In some embodiments, the detection sensitivity is at least about 0.79. In some embodiments, the detection sensitivity is at least about 0.8. In some embodiments, the detection sensitivity is at least about 0.81. In some embodiments, the detection sensitivity is at least about 0.82. In some embodiments, the detection sensitivity is at least about 0.83. In some embodiments, the detection sensitivity is at least about 0.84. In some embodiments, the detection sensitivity is at least about 0.85. In some embodiments, the detection sensitivity is at least about 0.86. In some embodiments, the detection sensitivity is at least about 0.87. In some embodiments, the detection sensitivity is at least about 0.88. In some embodiments, the detection sensitivity is at least about 0.89. In some embodiments, the detection sensitivity is at least about 0.9. In some embodiments, the detection sensitivity is at least about 0.91. In some embodiments, the detection sensitivity is at least about 0.92. In some embodiments, the detection sensitivity is at least about 0.93. In some embodiments, the detection sensitivity is at least about 0.94. In some embodiments, the detection sensitivity is at least about 0.95. In some embodiments, the detection sensitivity is at least about 0.96. In some embodiments, the detection sensitivity is at least about 0.97. In some embodiments, the detection sensitivity is at least about 0.98. In some embodiments, the detection sensitivity is at least about 0.99. In some embodiments, the detection sensitivity is at least about 1.

The method described herein may have a detection specificity. In some cases, the detection specificity of the method for HCC may be calculated by the following formula:

Specificity=Number of samples without HCC and identified to be negative/(Number of samples without HCC and identified to be positive+Number of samples without HCC and identified to be negative) or Specificity=Number of samples without HCC and identified to be positive/Total number of samples without HCC

In some embodiments, the detection specificity is at least about 0.5. In some embodiments, the detection specificity is at least about 0.51. In some embodiments, the detection specificity is at least about 0.52. In some embodiments, the detection specificity is at least about 0.53. In some embodiments, the detection specificity is at least about 0.54. In some embodiments, the detection specificity is at least about 0.55. In some embodiments, the detection specificity is at least about 0.56. In some embodiments, the detection specificity is at least about 0.57. In some embodiments, the detection specificity is at least about 0.58. In some embodiments, the detection specificity is at least about 0.59. In some embodiments, the detection specificity is at least about 0.6. In some embodiments, the detection specificity is at least about 0.61. In some embodiments, the detection specificity is at least about 0.62. In some embodiments, the detection specificity is at least about 0.63. In some embodiments, the detection specificity is at least about 0.64. In some embodiments, the detection specificity is at least about 0.65. In some embodiments, the detection specificity is at least about 0.66. In some embodiments, the detection specificity is at least about 0.67. In some embodiments, the detection specificity is at least about 0.68. In some embodiments, the detection specificity is at least about 0.69. In some embodiments, the detection specificity is at least about 0.7. In some embodiments, the detection specificity is at least about 0.71. In some embodiments, the detection specificity is at least about 0.72. In some embodiments, the detection specificity is at least about 0.73. In some embodiments, the detection specificity is at least about 0.74. In some embodiments, the detection specificity is at least about 0.75. In some embodiments, the detection specificity is at least about 0.76. In some embodiments, the detection specificity is at least about 0.77. In some embodiments, the detection specificity is at least about 0.78. In some embodiments, the detection specificity is at least about 0.79. In some embodiments, the detection specificity is at least about 0.8. In some embodiments, the detection specificity is at least about 0.81. In some embodiments, the detection specificity is at least about 0.82. In some embodiments, the detection specificity is at least about 0.83. In some embodiments, the detection specificity is at least about 0.84. In some embodiments, the detection specificity is at least about 0.85. In some embodiments, the detection specificity is at least about 0.86. In some embodiments, the detection specificity is at least about 0.87. In some embodiments, the detection specificity is at least about 0.88. In some embodiments, the detection specificity is at least about 0.89. In some embodiments, the detection specificity is at least about 0.9. In some embodiments, the detection specificity is at least about 0.91. In some embodiments, the detection specificity is at least about 0.92. In some embodiments, the detection specificity is at least about 0.93. In some embodiments, the detection specificity is at least about 0.94. In some embodiments, the detection specificity is at least about 0.95. In some embodiments, the detection specificity is at least about 0.96. In some embodiments, the detection specificity is at least about 0.97. In some embodiments, the detection specificity is at least about 0.98. In some embodiments, the detection specificity is at least about 0.99. In some embodiments, the detection specificity is at least about 1.

### EXAMPLES

These examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1. Diagnosing HCC Using Probes in Humans

In this experiment, the probes of the present application were shown to accurately detect the activity levels of proteases associated with hepatocellular carcinoma (HCC) in a fluid sample to diagnose HCC in a subject.

In this experiment, more than 600 peptide substrates were screened to identify a panel of 21 biosensors that interrogate multiple biological pathways implicated in HCC pathogenesis. This panel of biosensors was highly effective at detecting HCC, with AUCs (area under the ROC curve) above 0.94, when independently tested on two diverse cohorts of patient with known HCC vs. healthy controls. In this study the panel size was increased to 30 by adding 9 biosensors which were selected for their differential activity in the mice models of HCC vs fibrosis or fibrosis vs healthy.

Retrospective plasma samples were obtained from patients diagnosed with HCC or cirrhosis without HCC. Protease biosensor cleavage was assayed from plasma by fluorimetry, and the relative signal was used for classification by regularized logistic regression using 100 cross-validation (80% train, 20% validation splits).

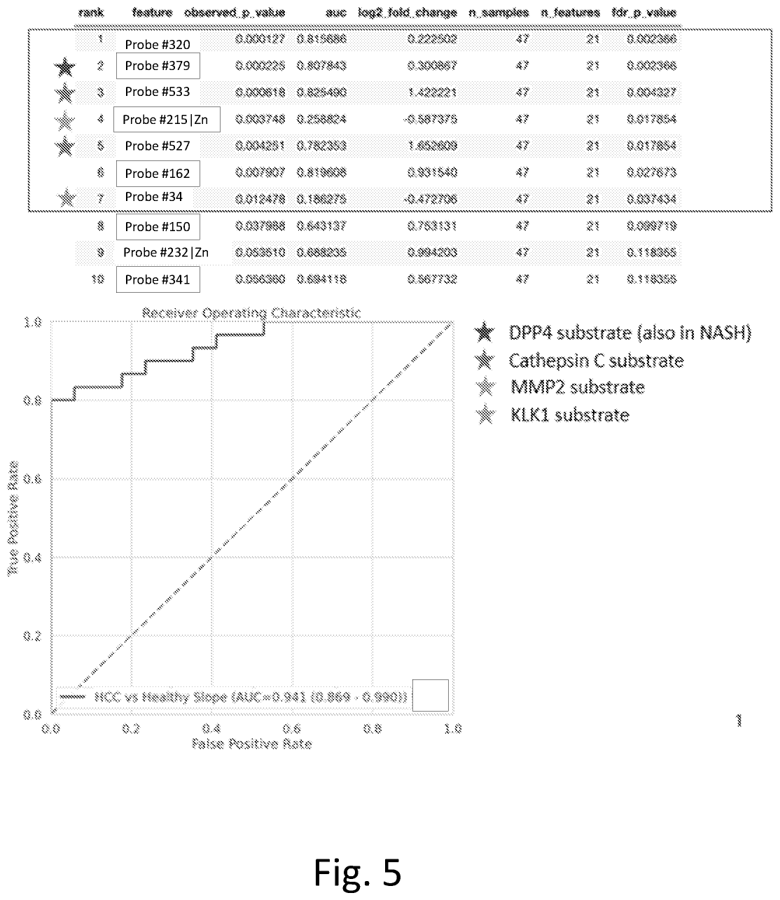

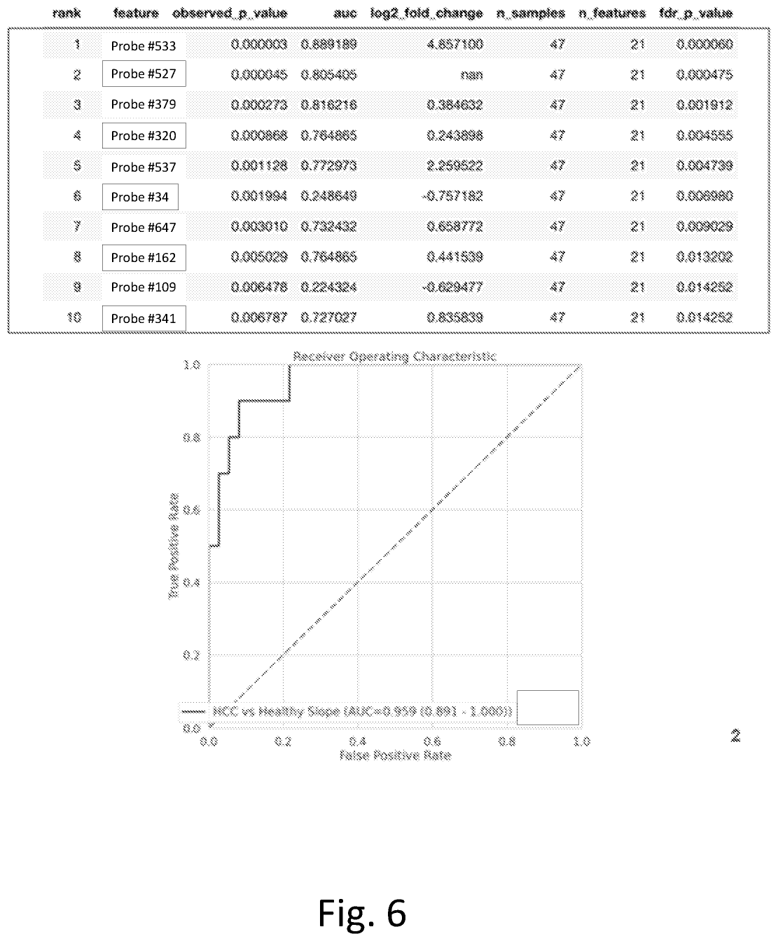

Protease activity levels associated with HCC were assessed in vitro in two groupings of human samples, one healthy and one with viral-induced HCC. Table 4 shows the demographic data of the samples, and results in FIG. 5 show that protease activity significantly discriminates HCC vs healthy samples. The experiment was repeated with life-style-induced HCC samples. Table 5 shows the demographic breakdown of the samples, and FIG. 6 shows that protease activity again significantly discriminates HCC vs healthy samples.

TABLE 4
Demographic data of Healthy and viral-induced HCC patients

| | HCC Samples | Healthy Samples |
|---|---|---|
| Total Number of Samples | 30 | 17 |
| Female:Male Ratio | 7:23 (23%:77%) | 7:10 (41%:59%) |
| Average Age | 55.8 ± 9.9 | 35.8 ± 12.6 |
| Average BMI | 22.3 ± 2.3 | 28.2 ± 4.5 |
| Ethnic Demographics | 29 Asian | 4 African |
| | 1 Caucasian | 3 Asian |
| | | 10 Caucasian |

TABLE 5
Demographic data of Healthy and lifestyle-induced HCC patients

| | HCC Samples | Healthy Samples |
|---|---|---|
| Total Number of Samples | 10 | 37 |
| Female:Male Ratio | 5:5 (50%:50%) | 13:24 (35%:65%) |
| Average Age | 59.0 ± 15.4 | 59.2 ± 9.4 |
| Average BMI | 23.6 ± 4.7 | 25.8 ± 2.9 |
| Ethnic Demographics | 1 Asian | 37 Caucasian |
| | 9 Caucasian | |

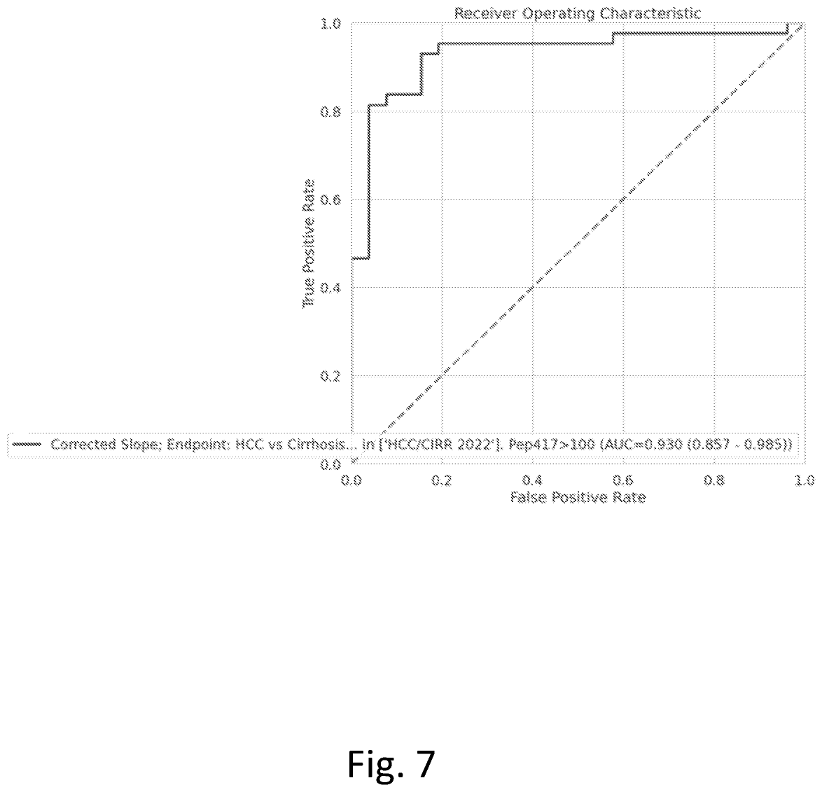

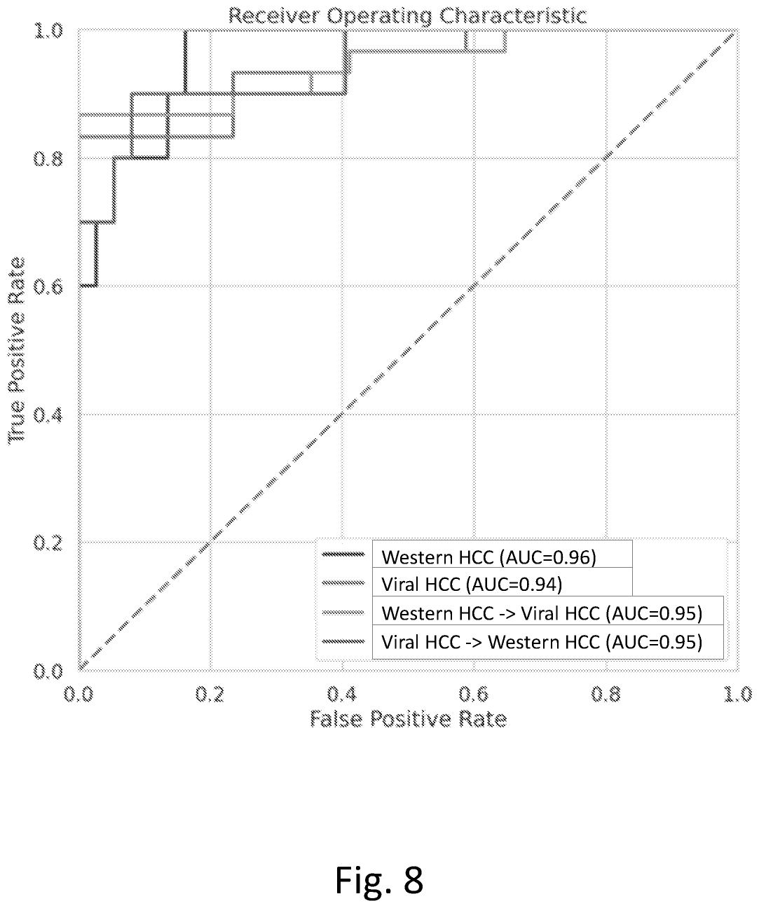

FIG. 7 shows plasma protease activity in 43 patients diagnosed with HCC (65% Male, mean age 58 years, 40% White/60% Asian, mean BMI 24 kg/m2, X etiology, 72% early stage I or II) were compared to 26 patients with underlying cirrhosis only (30% Male, mean age 57 years, 100% White, mean BMI 36 kg/m2, 100% NASH etiology). Protease biosensors were effective at differentiating HCC and cirrhosis (AUC=0.93 [CI 0.86-0.98]). Furthermore, using the best cut point under AUROC, the sensitivity and specificity were 0.84 and 0.85, respectively. The most significant biosensors in the classification of HCC vs cirrhosis are substrates of MMPs, DPP4, ANPEP, Aminopeptidase (ERAP1, ARTS-1), KLK14 and Cathepsins (L, S, K, B) and ADAM17. HCC signal generalizes across cohorts without classifier retaining (FIG. 8).

Thus, probes of the present application can accurately detect the activity levels of proteases associated with a biological condition or disease-state in a subject, ex vivo, using a body fluid sample. Novel non-invasive biosensors that measure protease activity are able to differentiate HCC (AUC 0.93) compared to background cirrhosis only. Consistent with other data in HCC vs. healthy and NASH vs. healthy, measurement of protease activity is a potentially important new diagnostic platform.

### Example 2: Detection of HCC Stage Using Protease Activity

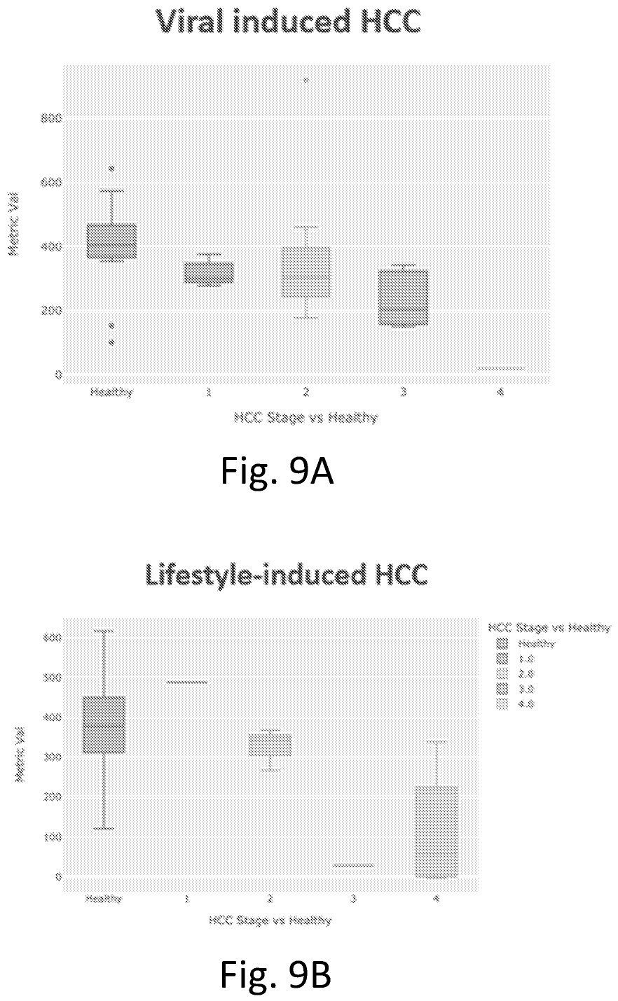

As shown in FIG. 9A-9B, protease activity is able to not only detect difference between healthy and diseased samples, but it can also differentiate among stages of HCC in both viral-induced (FIG. 9A) and lifestyle-induced HC samples (FIG. 9B). This experiment used the same methods as described in Example 1.

### Example 3: Plasma Protease Activity and Alpha Feto-Protein (AFP) to Detect HCC

HCC surveillance using ultrasound can be combined with detection of Alpha-Feto-Protein (AFP). In this experiment, AFP detection was combined with a protease biosensor biomarker panel to evaluate the efficacy of the combination treatment to distinguish patients with HCC from patients with cirrhosis without HCC.

Retrospective plasma samples were obtained from patients diagnosed with HCC (cases) and those with cirrhosis without HCC (controls). Protease biosensor cleavage was assayed from plasma by fluorimetry. The relative signal was used for classification by regularized logistic regression using 100 cross-validation (80% train, 20% validation splits). The maximum point from the Youden's J index in the training set was used to the define the optimal cut-off, which was used to calculate sensitivity and specificity for HCC detection in the validation sets. AFP was measured in plasma samples for all patients using a standard ELISA assay. AFP thresholds at both 20 ng/mL and 10 ng/mL were evaluated in this analysis.

Plasma protease activity and AFP levels were assessed in 54 HCC cases (76% male, mean age 56 years, 65% HBV, 82% TNM stage 1 or 2) and 23 cirrhosis controls (35% male, mean age 58 years, 100% NASH etiology). Our panel of protease biosensors had a high accuracy for differentiating patients with HCC vs those with cirrhosis (AUC 0.87 [95% CI 0.78-0.94]). The sensitivity and specificity of the assay, at its pre-specified cut-off, were 0.79 and 0.77, respectively. The sensitivity and specificity of AFP at a cut-off of 20 ng/mL were 0.44 and 1.00, respectively. Of note, the assay was able to detect HCC in 68% (22 of 30) of patients with AFP<20 ng/mL. Finally, a combined classifier of the assay and AFP>20 ng/mL had an AUC of 0.90 [95% CI 0.82-0.96] for differentiating HCC from cirrhosis. The sensitivity and specificity of the combined classifier for HCC detection were 0.82 and 0.82, respectively. Similar results were observed when the AFP threshold was adjusted to the more stringent cutoff of 10 ng/mL.

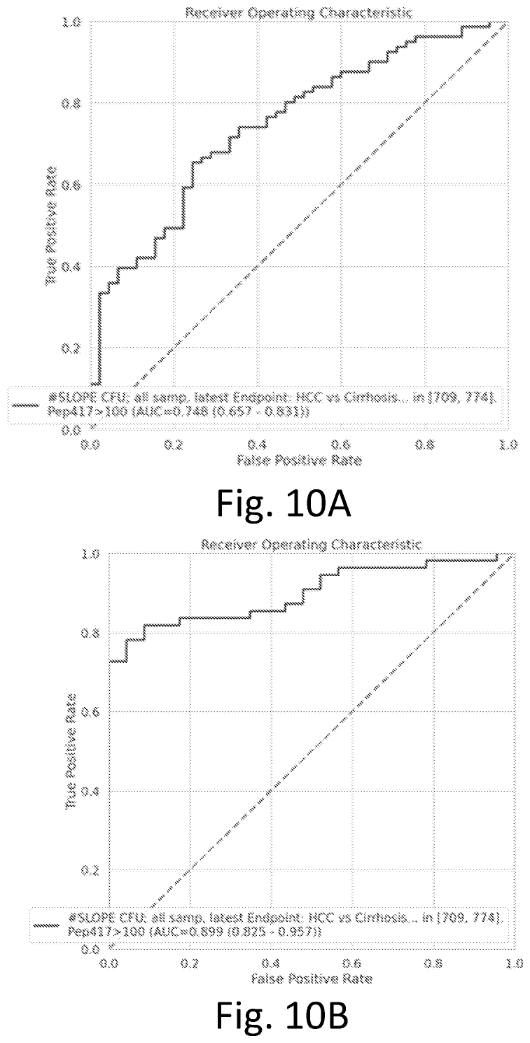

As seen in FIG. 10A-B, the protease biosensor panel assay was able to correctly classify as HCC 68% (17 out 25) of patients with low AFP levels (AFP<10 ng/ml). Moreover, classification by regularized logistic regression with 100-fold cross-validation (80% train, 20% validation splits) using the protease biosensor panel assay in the challenging subset of patients with low AFP levels (AFP, 10 ng/ml) showed an AUC of 0.75. Finally, a combined classifier of the protease biosensor panel assay and AFP had an AUC of 0.90 [CI 0.83-0.96] for differentiating HCC from cirrhosis in this cohort of patients (FIG. 10B). Using the Youden's cut-off, sensitivity and specificity for HCC detection of this combined classifier of protease biosensor panel plus AFP were 0.84 and 0.87, respectively. These combined results demonstrate the utility of using novel non-invasive biosensors measuring protease activity independently or in combination with AFP to accurately differentiate HCC from cirrhosis.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method comprising:
contacting a body fluid sample from a subject with a synthetic molecule, wherein the contacting occurs ex vivo,
wherein the subject has or is suspected of having cirrhosis of a liver of the subject,
wherein the synthetic molecule comprises a cleavable linker, a reporter, and a carrier, and
wherein the synthetic molecule reacts with an enzyme from the body fluid, causing the reporter to form a detectable signal,
detecting the detectable signal,
wherein the detection indicates whether the subject has hepatocellular carcinoma (HCC),
wherein the detection comprises a detection sensitivity of at least 0.5.

2. The method of claim 1, wherein the detection sensitivity is at least 0.8.

3. The method of claim 1, wherein the detection sensitivity is calculated by dividing a number of samples with HCC and identified to be positive by a total number of samples, wherein the total number of samples comprises the sum of the samples with HCC and identified to be positive and samples with HCC and identified to be negative.

4. The method of claim 1, wherein the HCC comprises a stage of HCC.

5. The method of claim 1, wherein the enzyme from the body fluid cleaves the cleavable linker, thereby releasing the reporter from the synthetic molecule, wherein the released reporter forms the detectable signal.

6. The method of claim 1, wherein the cleavable linker comprises a peptide.

7. The method of claim 1, wherein the synthetic molecule comprises a panel of peptides.

8. The method of claim 7, wherein the panel of peptides comprises at least 10 different peptides.

9. The method of claim 1, wherein the enzyme comprises a protease.

10. The method of claim 9, wherein the protease comprises a matrix metalloprotease (MMP), a dipeptidyl-peptidase 4 (DPP4), an alanyl aminopeptidase (ANPEP), Aminopeptidase (ERAP1, ARTS-1), a Kallikrein related peptidase 14 (KLK14), a Cathepsin or an ADAM17.

11. The method of claim 1, wherein the reporter comprises a fluorescent label.

12. The method of claim 11, wherein the fluorescent label comprises a 5-carboxyfluorescein (5-FAM), a 7-amino-4-carbamoylmethylcoumarin (ACC), a 7-Amino-4-methylcoumarin (AMC), a 2-Aminobenzoyl (Abz), a Cy7, a Cy5, a Cy3, or a (5-((2-Aminoethyl)amino)naphthalene-1-sulfonic acid) (EDANS).

13. The method of claim 1, wherein the synthetic molecule further comprises a fluorescent quencher.

14. The method of claim 13, wherein the fluorescent quencher comprises BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl]azo)benzoyl), Dnp (2,4-dinitrophenyl), or Eclipse.

15. The method of claim 1, wherein the body fluid sample comprises blood, plasma, bone marrow fluid, lymphatic fluid, bile, amniotic fluid, mucosal fluid, saliva, urine, cerebrospinal fluid, spinal fluid, synovial fluid, semen, ductal aspirate, feces, stool, vaginal effluent, lachrymal fluid, tissue lysate or patient-derived cell line supernatant.

16. The method of claim 1, wherein the body fluid sample comprises a rinse fluid, a conditioning media or buffer, a swab viral transport media, a saline, a culture media, or a cell culture supernatant.

17. The method of claim 16, wherein the rinse fluid comprises a mouthwash rinse, a bronchioalveolar rinse, a lavage fluid, a hair wash rinse, a nasal spray effluent, a swab of any bodily surface, orifice, organ structure, or solid tumor biopsies applied to saline or any media, or any derivatives thereof.

18. The method of claim 1, wherein the body fluid sample comprises an alpha fetoprotein (AFP) level of less than about 10 ng/ml.

19. The method of claim 1 or claim 10, where in the detection further comprises detecting the AFP level of the body fluid sample.

20. The method of claim 1, wherein the detection comprises detecting a rate of formation or an amount of the detectable signal.

21. The method of claim 1, wherein the subject comprises a mammal.

22. The method of claim 1, wherein the subject comprises a human.
